SEQUENCE LISTING (174 sequences)

SEQ ID NO: 1 - DNA, 321 bp, Homo sapiens
  CDS (1)..(321)
gacattcagatgacccagtctcctgcctcccagtctgcatctctgggagaaagtgtcaccatcacatgcctggcaagtcagaccattgatacatggttagcatggtatcagcagaaaccagggaaatctcctcagctcctgatttatgctgccaccaacttggcagatggggtcccatcaaggttcagtggcagtggatctggcacaaaattttctttcaagatcagcagcctacaggctgaagattttgtaaattattactgtcaacaagtttacagttctccattcacgttcggtgctgggaccaagctggagctgaaa

SEQ ID NO: 2 - protein, 107 aa, Homo sapiens
DIQMTQSPASQSASLGESVTITCLASQTIDTWLAWYQQKPGKSPQLLIYAATNLADGVPSRFSGSGSGTKFSFKISSLQAEDFVNYYCQQVYSSPFTFGAGTKLELK

SEQ ID NO: 3 - DNA, 351 bp, Homo sapiens
  CDS (1)..(351)
gagatccagctgcagcagtctggacctgagctggtgaagcctggggcttcagtgcaggtatcctgcaagacttctggttactcattcactgactacaacgtgtactgggtgaggcagagccatggaaagagccttgagtggattggatatattgatccttacaatggtattactatctacgaccagaacttcaagggcaaggccacattgactgttgacaagtcttccaccacagccttcatgcatctcaacagcctgacatctgacgactctgcagtttatttctgtgcaagagatgtgactacggcccttgacttctggggccaaggcaccactctcacagtctcctca

SEQ ID NO: 4 - protein, 117 aa, Homo sapiens
EIQLQQSGPELVKPGASVQVSCKTSGYSFTDYNVYWVRQSHGKSLEWIGYIDPYNGITIYDQNFKGKATLTVDKSSTTAFMHLNSLTSDDSAVYFCARDVTTALDFWGQGTTLTVSS

SEQ ID NO: 5 - protein, 7 aa, Homo sapiens
LASQTID

SEQ ID NO: 6 - protein, 7 aa, Homo sapiens
AATNLAD

SEQ ID NO: 7 - protein, 9 aa, Homo sapiens
QQVYSSPFT

SEQ ID NO: 8 - protein, 6 aa, Homo sapiens
TDYNVY

SEQ ID NO: 9 - protein, 17 aa, Homo sapiens
YIDPYNGITIYDQNFKG

SEQ ID NO: 10 - protein, 8 aa, Homo sapiens
DVTTALDF

SEQ ID NO: 11 - DNA, 21 bp, Homo sapiens
ctggcaagtcagaccattgat

SEQ ID NO: 12 - DNA, 21 bp, Homo sapiens
gctgccaccaacttggcagat

SEQ ID NO: 13 - DNA, 28 bp, Homo sapiens
caacaagtttacagttctccattcacgt

SEQ ID NO: 14 - DNA, 18 bp, Homo sapiens
actgactacaacgtgtac

SEQ ID NO: 15 - DNA, 51 bp, Homo sapiens
tatattgatccttacaatggtattactatctacgaccagaacttcaagggc

SEQ ID NO: 16 - DNA, 24 bp, Homo sapiens
gatgtgactacggcccttgacttc

SEQ ID NO: 17 - DNA, 23 bp, Artificial Sequence
  Description of Artificial Sequence Primer
gcacctccagatgttaactgctc

SEQ ID NO: 18 - DNA, 20 bp, Artificial Sequence
  Description of Artificial Sequence Primer
gaartavcccttgaccaggc

SEQ ID NO: 19 - DNA, 35 bp, Artificial Sequence
  Description of Artificial Sequence Primer
ggaggcggcggttctgacattgtgmtgwcmcartc

SEQ ID NO: 20 - DNA, 45 bp, Artificial Sequence
  Description of Artificial Sequence Primer
atttcaggcccagccggccatggccgargtycarctkcarcaryc

SEQ ID NO: 21 - DNA, 33 bp, Artificial Sequence
  Description of Artificial Sequence Primer
cccgggccaccatgkccccwrctcagytyctkg

SEQ ID NO: 22 - DNA, 35 bp, Artificial Sequence
  Description of Artificial Sequence Primer
cccgggccaccatggratgsagctgkgtmatsctc

SEQ ID NO: 23 - DNA, 52 bp, Artificial Sequence
  Description of Artificial Sequence Primer
atatactcgcgacagctacaggtgtccactccgagatccagctgcagcagtc

SEQ ID NO: 24 - DNA, 31 bp, Artificial Sequence
  Description of Artificial Sequence Primer
gacctgaattctaaggagactgtgagagtgg

SEQ ID NO: 25 - DNA, 29 bp, Artificial Sequence
  Description of Artificial Sequence Primer
ttaattgatatccagatgacccagtctcc

SEQ ID NO: 26 - DNA, 45 bp, Artificial Sequence
  Description of Artificial Sequence Primer
taatcgttcgaaaagtgtacttacgtttcagctccagcttggtcc

SEQ ID NO: 27 - protein, 38 aa, Homo sapiens
DIQMTQSPASLSASVGDRVTITCWYLQKPGKSPQLLIY

SEQ ID NO: 28 - protein, 42 aa, Homo sapiens
GVPSRFSGSGSGTDFSFTISSLQPEDFATYYCFGQGTKLEIK

SEQ ID NO: 29 - protein, 44 aa, Homo sapiens
QIQLVQSGGEVKKPGASVRVSCKASGYSFTWVRQSPGKGLEWIG

SEQ ID NO: 30 - protein, 43 aa, Homo sapiens
KATLTVDKSTSTAYMELSSLRSEDTAVYFCARWGQGTTVTVSS

SEQ ID NO: 31 - DNA, 37 bp, Artificial Sequence
  Description of Artificial Sequence Primer
tttcgtacgtcttgtcccagatccagctgcagcagtc

SEQ ID NO: 32 - DNA, 43 bp, Homo sapiens
agcgaattctgaggagactgtgacagtggtgccttggccccag

SEQ ID NO: 33 - DNA, 38 bp, Artificial Sequence
  Description of Artificial Sequence Primer
gtgaggcagagccctggaaagggccttgagtggattgg

SEQ ID NO: 34 - DNA, 38 bp, Artificial Sequence
  Description of Artificial Sequence Primer
ccaatccactcaaggccctttccagggctctgcctcac

SEQ ID NO: 35 - DNA, 47 bp, Artificial Sequence
  Description of Artificial Sequence Primer
gcatctcaacagcctgagatctgaagacactgcagtttatttctgtg

SEQ ID NO: 36 - DNA, 43 bp, Artificial Sequence
  Description of Artificial Sequence Primer
ctgcagtgtcttcagatctcaggctgttgagatgcatgaaggc

SEQ ID NO: 37 - DNA, 43 bp, Artificial Sequence
  Description of Artificial Sequence Primer
gtcttcagatctcaggctgctgagctccatgaaggctgtggtg

SEQ ID NO: 38 - DNA, 25 bp, Artificial Sequence
  Description of Artificial Sequence Primer
tacgactcactatagggcgaattgg

SEQ ID NO: 39 - DNA, 43 bp, Artificial Sequence
  Description of Artificial Sequence Primer
ctgttgacaagtctaccagcacagcctacatggagctcagcag

SEQ ID NO: 40 - DNA, 43 bp, Artificial Sequence
  Description of Artificial Sequence Primer
ctgctgagctccatgtaggctgtgctggtagacttgtcaacag

SEQ ID NO: 41 - DNA, 47 bp, Artificial Sequence
  Description of Artificial Sequence Primer
gcactgaagccccaggcttcaccagctcacctccagactgctgcagc

SEQ ID NO: 42 - DNA, 49 bp, Artificial Sequence
  Description of Artificial Sequence Primer
ctggggcttcagtgcgggtatcctgcaaggcttctggttactcattcac

SEQ ID NO: 43 - DNA, 46 bp, Artificial Sequence
  Description of Artificial Sequence Primer
tcgtacgtcttgtcccagatccagctggtgcagtctggaggtgagc

SEQ ID NO: 44 - DNA, 44 bp, Artificial Sequence
  Description of Artificial Sequence Primer
gcactgaagccccaggcttcttcacctcacctccagactgcacc

SEQ ID NO: 45 - DNA, 32 bp, Artificial Sequence
  Description of Artificial Sequence Primer
gcagtctggacctgagctgaagaagcctgggg

SEQ ID NO: 46 - DNA, 32 bp, Artificial Sequence
  Description of Artificial Sequence Primer
ccccaggcttcttcagctcaggtccagactgc

SEQ ID NO: 47 - DNA, 33 bp, Artificial Sequence
  Description of Artificial Sequence Primer
gctggtgcagtctggacctgaggtgaagaagcc

SEQ ID NO: 48 - DNA, 33 bp, Artificial Sequence
  Description of Artificial Sequence Primer
ggcttcttcacctcaggtccagactgcaccagc

SEQ ID NO: 49 - DNA, 36 bp, Artificial Sequence
  Description of Artificial Sequence Primer
gcagtctggacctgagctggtgaagcctggggcttc

SEQ ID NO: 50 - DNA, 36 bp, Artificial Sequence
  Description of Artificial Sequence Primer
gaagccccaggcttcaccagctcaggtccagactgc

SEQ ID NO: 51 - DNA, 30 bp, Artificial Sequence
  Description of Artificial Sequence Primer
cagtctggacctgaggtggtgaagcctggg

SEQ ID NO: 52 - DNA, 30 bp, Artificial Sequence
  Description of Artificial Sequence Primer
cccaggcttcaccacctcaggtccagactg

SEQ ID NO: 53 - DNA, 42 bp, Artificial Sequence
  Description of Artificial Sequence Primer
ttcgaaaagtgtacttacgtttgatctccagcttggtcccag

SEQ ID NO: 54 - DNA, 29 bp, Artificial Sequence
  Description of Artificial Sequence Primer
accggtgatatccagatgacccagtctcc

SEQ ID NO: 55 - DNA, 29 bp, Artificial Sequence
  Description of Artificial Sequence Primer
ggttagcatggtatctgcagaaaccaggg

SEQ ID NO: 56 - DNA, 29 bp, Artificial Sequence
  Description of Artificial Sequence Primer
ccctggtttctgcagataccatgctaacc

SEQ ID NO: 57 - DNA, 25 bp, Artificial Sequence
  Description of Artificial Sequence Primer
tacgactcactatagggcgaattgg

SEQ ID NO: 58 - DNA, 31 bp, Artificial Sequence
  Description of Artificial Sequence Primer
ccacagatgcagacagggaggcaggagactg

SEQ ID NO: 59 - DNA, 51 bp, Artificial Sequence
  Description of Artificial Sequence Primer
ttcgaaaagtgtacttacgtttgatctccagcttggtaccagcaccgaacg

SEQ ID NO: 60 - DNA, 40 bp, Artificial Sequence
  Description of Artificial Sequence Primer
cctgtctgcatctgtgggagatagggtcaccatcacatgc

SEQ ID NO: 61 - DNA, 36 bp, Artificial Sequence
  Description of Artificial Sequence Primer
gatctccagcttggtaccctgaccgaacgtgaatgg

SEQ ID NO: 62 - DNA, 40 bp, Artificial Sequence
  Description of Artificial Sequence Primer
gtaggctgctgatcgtgaaagaaaagtctgtgccagatcc

SEQ ID NO: 63 - DNA, 47 bp, Artificial Sequence
  Description of Artificial Sequence Primer
cacgatcagcagcctacagcctgaagattttgtaaattattactgtc

SEQ ID NO: 64 - DNA, 45 bp, Artificial Sequence
  Description of Artificial Sequence Primer
gcagcctacagcctgaagattttgcaacttattactgtcaacaag

SEQ ID NO: 65 - DNA, 45 bp, Artificial Sequence
  Description of Artificial Sequence Primer
cttgttgacagtaataagttgcaaaatcttcaggctgtaggctgc

SEQ ID NO: 66 - DNA, 44 bp, Artificial Sequence
  Description of Artificial Sequence Primer
cagcagcctacagcctgaagattttgcaaattattactgtcaac

SEQ ID NO: 67 - DNA, 44 bp, Artificial Sequence
  Description of Artificial Sequence Primer
gttgacagtaataatttgcaaaatcttcaggctgtaggctgctg

SEQ ID NO: 68 - DNA, 40 bp, Artificial Sequence
  Description of Artificial Sequence Primer
cagtggatctggcacaaagttttctttcacgatcagcagc

SEQ ID NO: 69 - DNA, 40 bp, Artificial Sequence
  Description of Artificial Sequence Primer
gctgctgatcgtgaaagaaaactttgtgccagatccactg

SEQ ID NO: 70 - DNA, 33 bp, Artificial Sequence
  Description of Artificial Sequence Primer
ctgcagaaaccagggcaatctcctcagctcctg

SEQ ID NO: 71 - DNA, 33 bp, Artificial Sequence
  Description of Artificial Sequence Primer
caggagctgaggagattgccctggtttctgcag

SEQ ID NO: 72 - protein, 79 aa, Homo sapiens
DIQMTQSPASQSASLGESVTITCWYQQKPGKSPQLIYGVPSRFSGSGSGTKFSFKISSLQAEDFVNYYCFGAGTKLELK

SEQ ID NO: 73 - protein, 79 aa, Homo sapiens
DIQMTQSPASQSASLGESVTITCWYQQKPGKSPQLIYGVPSRFSGSGSGTKFSFKISSLQAEDFVNYYCFGAGTKLEIK

SEQ ID NO: 74 - protein, 79 aa, Homo sapiens
DIQMTQSPASQSASLGESVTITCWYLQKPGKSPQLIYGVPSRFSGSGSGTKFSFKISSLQAEDFVNYYCFGAGTKLEIK

SEQ ID NO: 75 - protein, 79 aa, Homo sapiens
DIQMTQSPASLSASVGDRVTITCWYLQKPGKSPQLIYGVPSRFSGSGSGTKFSFKISSLQAEDFVNYYCFGQGTKLEIK

SEQ ID NO: 76 - protein, 79 aa, Homo sapiens
DIQMTQSPASQSASLGESVTITCWYLQKPGKSPQLIYGVPSRFSGSGSGTKFSFKISSLQAEDFVNYYCFGQGTKLEIK

SEQ ID NO: 77 - protein, 79 aa, Homo sapiens
DIQMTQSPASQSASLGESVTITCWYLQKPGKSPQLIYGVPSRFSGSGSGTDFSFTISSLQPEDFVNYYCFGQGTKLEIK

SEQ ID NO: 78 - protein, 79 aa, Homo sapiens
DIQMTQSPASQSASLGESVTITCWYLQKPGKSPQLIYGVPSRFSGSGSGTDFSFTISSLQPEDFATYYCFGQGTKLEIK

SEQ ID NO: 79 - protein, 79 aa, Homo sapiens
DIQMTQSPASLSASVGDRVTITCWYLQKPGKSPQLIYGVPSRFSGSGSGTDFSFTISSLQPEDFATYYCFGQGTKLEIK

SEQ ID NO: 80 - protein, 79 aa, Homo sapiens
DIQMTQSPASLSASVGDRVTITCWYLQKPGKSPQLIYGVPSRFSGSGSGTDFSFTISSLQPEDFANYYCFGQGTKLEIK

SEQ ID NO: 81 - protein, 79 aa, Homo sapiens
DIQMTQSPASLSASVGDRVTITCWYLQKPGKSPQLIYGVPSRFSGSGSGTKFSFTISSLQPEDFANYYCFGQGTKLEIK

SEQ ID NO: 82 - protein, 79 aa, Homo sapiens
DIQMTQSPASLSASVGDRVTITCWYLQKPGQSPQLIYGVPSRFSGSGSGTKFSFTISSLQPEDFANYYCFGQGTKLEIK

SEQ ID NO: 83 - protein, 87 aa, Homo sapiens
EIQLQQSGPELVKPGASVQVSCKTSGYSFTWVRQSHGKSLEWIGKATLTVDKSSTTAFMHLNSLTSDDSAVYFCARWGQGTTLTVSS

SEQ ID NO: 84 - protein, 87 aa, Homo sapiens
QIQLQQSGPELVKPGASVQVSCKTSGYSFTWVRQSHGKSLEWIGKATLTVDKSSTTAFMHLNSLTSDDSAVYFCARWGQGTTVTVSS

SEQ ID NO: 85 - protein, 87 aa, Homo sapiens
QIQLQQSGPELVKPGASVQVSCKTSGYSFTWVRQSPGKGLEWIGKATLTVDKSSTTAFMHLNSLTSDDSAVYFCARWGQGTTVTVSS

SEQ ID NO: 86 - protein, 87 aa, Homo sapiens
QIQLQQSGPELVKPGASVQVSCKTSGYSFTWVRQSPGKGLEWIGKATLTVDKSSTTAFMHLNSLRSEDTAVYFCARWGQGTTVTVSS

SEQ ID NO: 87 - protein, 87 aa, Homo sapiens
QIQLQQSGPELVKPGASVQVSCKTSGYSFTWVRQSPGKGLEWIGKATLTVDKSSTTAFMELSSLRSEDTAVYFCARWGQGTTVTVSS

SEQ ID NO: 88 - protein, 87 aa, Homo sapiens
QIQLQQSGPELVKPGASVQVSCKTSGYSFTWVRQSPGKGLEWIGKATLTVDKSTSTAYMELSSLRSEDTAVYFCARWGQGTTVTVSS

SEQ ID NO: 89 - protein, 87 aa, Homo sapiens
QMQLQQSGGELVKPGASVRVSCKASGYSFTWVRQSPGKGLEWIGKATLTVDKSTSTAYMELSSLRSEDTAVYFCARWGQGTTVTVSS

SEQ ID NO: 90 - protein, 87 aa, Homo sapiens
QIQLVQSGGELVKPGASVRVSCKASGYSFTWVRQSPGKGLEWIGKATLTVDKSTSTAYMELSSLRSEDTAVYFCARWGQGTTVTVSS

SEQ ID NO: 91 - protein, 87 aa, Homo sapiens
QIQLVQSGGEVKKPGASVRVSCKASGYSFTWVRQSPGKGLEWIGKATLTVDKSTSTAYMELSSLRSEDTAVYFCARWGQGTTVTVSS

SEQ ID NO: 92 - protein, 87 aa, Homo sapiens; identical to SEQ ID NO: 91

SEQ ID NO: 93 - protein, 87 aa, Homo sapiens
QIQLVQSGPEVKKPGASVRVSCKASGYSFTWVRQSPGKGLEWIGKATLTVDKSTSTAYMELSSLRSEDTAVYFCARWGQGTTVTVSS

SEQ ID NO: 94 - protein, 87 aa, Homo sapiens
QIQLVQSGPELKKPGASVRVSCKASGYSFTWVRQSPGKGLEWIGKATLTVDKSTSTAYMELSSLRSEDTAVYFCARWGQGTTVTVSS

SEQ ID NO: 95 - protein, 87 aa, Homo sapiens
QIQLVQSGPELVKPGASVRVSCKASGYSFTWVRQSPGKGLEWIGKATLTVDKSTSTAYMELSSLRSEDTAVYFCARWGQGTTVTVSS

SEQ ID NO: 96 - protein, 87 aa, Homo sapiens
QIQLVQSGPEVVKPGASVRVSCKASGYSFTWVRQSPGKGLEWIGKATLTVDKSTSTAYMELSSLRSEDTAVYFCARWGQGTTVTVSS

SEQ ID NO: 97 - protein, 107 aa, Homo sapiens [sequence omitted]

SEQ ID NO: 98 - protein, 332 aa, Homo sapiens
EFASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 107 aa, Homo sapiens [sequence omitted]

SEQ ID NO: 100 - protein, 329 aa, Homo sapiens
EFASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 17 aa, Homo sapiens
YIDPYNGITIYDQNLKG

SEQ ID NO: 102 - protein, 38 aa, Homo sapiens
DIQMTQSPASQSASLGESVTITCWYQQKPGKSPQLLIY

SEQ ID NO: 103 - protein, 11 aa, Homo sapiens
LASQTIDTWLA

SEQ ID NO: 104 - protein, 5 aa, Homo sapiens
DYNVY

SEQ ID NO: 105 - DNA, 324 bp, Homo sapiens
  CDS (1)..(324)
gatatcgttctctcccagtctccagcaatcctgtctgcatctccaggggaaaaggtcacaatgacttgcagggccagctcaagtgtaaattacatgcactggtaccagcagaagccaggatcctcccccaaaccctggatttctgccacatccaaccttctggctggagtccctgctcgcttcagtggcagtgggtctgggacctcttactctctcacaatcagcagagtggaggctgaagatgctgccacttattactgccagcagtggagtagtaacccacccacgttcggaggggggaccatgctggaaataaaacgtaag

SEQ ID NO: 106 - protein, 108 aa, Homo sapiens
DIVLSQSPAILSASPGEKVTMTCRASSSVNYMHWYQQKPGSSPKPWISATSNLLAGVPARFSGSGSGTSYSLTISRVEAEDAATYYCQQWSSNPPTFGGGTMLEIKRK

SEQ ID NO: 107 - DNA, 369 bp, Homo sapiens
  CDS (1)..(369)
gaagtgatgctggtggagtctggtggaggattggtgcagcctaaagggtcattgaaactctcatgtgcagcctctggattcaccttcaataactacgccatgaattgggtccgccaggctccaggaaagggtttggaatgggttgctcgcataagaagtaaaagtaataattatgcaacattttatgccgattcagtgaaagacaggttcaccatctccagagatgattcacaaagcatgctctatctgcaaatgaacaacttgaaaactgaggacacagccatgtattactgtgtgagacggggggcttcagggattgactatgctatggactactggggtcaaggaacctcactcaccgtctcctca

SEQ ID NO: 108 - protein, 123 aa, Homo sapiens
EVMLVESGGGLVQPKGSLKLSCAASGFTFNNYAMNWVRQAPGKGLEWVARIRSKSNNYATFYADSVKDRFTISRDDSQSMLYLQMNNLKTEDTAMYYCVRRGASGIDYAMDYWGQGTSLTVSS

SEQ ID NO: 109 - protein, 80 aa, Homo sapiens
DIVLSQSPAILSASPGEKVTMTCWYQQKPGSSPKPWISGVPARFSGSGSGTSYSLTISRVEAEDAATYYCFGGGTMLEIK

SEQ ID NO: 110 - protein, 80 aa, Homo sapiens
QIVLTQSPAILSASPGEKVTMTCWYQQKPGSSPKPWISGVPARFSGSGSGTSYSLTISRVEAEDAATYYCFGGGTKLEIK

SEQ ID NO: 111 - protein, 80 aa, Homo sapiens
QIVLTQSPAILSASPGEKVTMTCWYQQKPGQSPKPWISGVPARFSGSGSGTSYSLTISRVEAEDAATYYCFGGGTKLEIK

SEQ ID NO: 112 - protein, 80 aa, Homo sapiens
QIVLTQSPAILSASPGEKVTMTCWYQQKPGSSPKPWISGVPARFSGSGSGTSYSLTISAMEAEDAATYYCFGGGTKLEIK

SEQ ID NO: 113 - protein, 80 aa, Homo sapiens
QIVLTQSPAILSASPGEKVTMTCWYQQKPGQSPKPWISGVPARFSGSGSGTSYSLTISAMEAEDAATYYCFGGGTKLEIK

SEQ ID NO: 114 - protein, 80 aa, Homo sapiens
QIVLTQSPAIMSASPGEKVTITCWYQQKPGQSPKPWISGVPARFSGSGSGTSYSLTISAMEAEDAATYYCFGGGTKLEIK

SEQ ID NO: 115 - protein, 10 aa, Homo sapiens
RASSSVNYMH

SEQ ID NO: 116 - protein, 7 aa, Homo sapiens
ATSNLAS

SEQ ID NO: 117 - protein, 9 aa, Homo sapiens
QQWSSNPPT

SEQ ID NO: 118 - protein, 82 aa, Homo sapiens
EVMLVESGGGLVQPKGSLKLSCAASWVRQAPGKGLEWVARFTISRDDSQSMLYLQMNNLKTEDTAMYYCVRWGQGTSLTVSS

SEQ ID NO: 119 - protein, 82 aa, Homo sapiens
EVQLVESGGGLVQPKGSLKLSCAASWVRQAPGKGLEWVARFTISRDDSQSMLYLQMNNLKTEDTAMYYCVRWGQGTSVTVSS

SEQ ID NO: 120 - protein, 82 aa, Homo sapiens
EVQLVESGGGLVQPKGSLKLSCAASWVRQAPGKGLEWVARFTISRDDSQSMLYLQMNSLKTEDTAVYYCVRWGQGTSVTVSS

SEQ ID NO: 121 - protein, 82 aa, Homo sapiens
EVQLVESGGGLVQPKGSLKLSCAASWVRQAPGKGLEWVARFTISRDDSQSMLYLQMNSLKTEDTAVYYCARWGQGTSVTVSS

SEQ ID NO: 122 - protein, 82 aa, Homo sapiens
EVQLVESGGGLVQPGGSLKLSCAASWVRQAPGKGLEWVARFTISRDDSQSMLYLQMNSLKTEDTAVYYCARWGQGTSVTVSS

SEQ ID NO: 123 - protein, 82 aa, Homo sapiens
EVQLVESGGGLVQPGGSLKLSCAASWVRQAPGKGLEWVARFTISRDDSKNSLYLQMNSLKTEDTAVYYCARWGQGTSVTVSS

SEQ ID NO: 124 - protein, 10 aa, Homo sapiens
GFTFNNYAMN

SEQ ID NO: 125 - protein, 19 aa, Homo sapiens
RIRSKSNNYATFYADSVKD

SEQ ID NO: 126 - protein, 12 aa, Homo sapiens
RGASGIDYAMDY

SEQ ID NO: 127 - protein, 80 aa, Homo sapiens
QIVLTQSPAIMSASPGDKVTITCWYQQKPGQSPKPWISGVPARFSGSGSGTSYSLTISAMEAEDAATYYCFGGGTKLEIK

SEQ ID NO: 128 - DNA, 20 bp, Artificial Sequence
  Description of Artificial Sequence Synthetic oligonucleotide
ggagacccaagcttgttaac

SEQ ID NO: 129 - DNA, 19 bp, Artificial Sequence
  Description of Artificial Sequence Synthetic oligonucleotide
cccagaggtgctcttggag

SEQ ID NO: 130 - DNA, 42 bp, Artificial Sequence
  Description of Artificial Sequence Synthetic oligonucleotide
tgttttcgtacgtcttgtccgaagtgcagctggtggagtctg

SEQ ID NO: 131 - DNA, 47 bp, Artificial Sequence
  Description of Artificial Sequence Synthetic oligonucleotide
gaacagcttgaaaactgaggacacagccgtgtattactgtgtgagac

SEQ ID NO: 132 - DNA, 53 bp, Artificial Sequence
  Description of Artificial Sequence Synthetic oligonucleotide
aatacacggctgtgtcctcagttttcaagctgttcatttgcagatagagcatg

SEQ ID NO: 133 - DNA, 44 bp, Artificial Sequence
  Description of Artificial Sequence Synthetic oligonucleotide
aattctgaattctgaggagacggtcactgaggttccttgacccc

SEQ ID NO: 134 - DNA, 34 bp, Artificial Sequence
  Description of Artificial Sequence Synthetic oligonucleotide
cagccgtgtattactgtgcgagacggggggcttc

SEQ ID NO: 135 - DNA, 34 bp, Artificial Sequence
  Description of Artificial Sequence Synthetic oligonucleotide
gaagccccccgtctcgcacagtaatacacggctg

SEQ ID NO: 136 - DNA, 35 bp, Artificial Sequence
  Description of Artificial Sequence Synthetic oligonucleotide
ggattggtgcagcctggcgggtcattgaaactctc

SEQ ID NO: 137 - DNA, 35 bp, Artificial Sequence
  Description of Artificial Sequence Synthetic oligonucleotide
gagagtttcaatgacccgccaggctgcaccaatcc

SEQ ID NO: 138 - DNA, 39 bp, Artificial Sequence
  Description of Artificial Sequence Synthetic oligonucleotide
gaggctctgcacaaccgcttcacgcagaagagcctctcc

SEQ ID NO: 139 - DNA, 39 bp, Artificial Sequence
  Description of Artificial Sequence Synthetic oligonucleotide
ggagaggctcttctgcgtgaagcggttgtgcagagcctc

SEQ ID NO: 140 - DNA, 39 bp, Artificial Sequence
  Description of Artificial Sequence Synthetic oligonucleotide
gattcaaaaaacagcctctatctgcaaatgaacaacttg

SEQ ID NO: 141 - DNA, 38 bp, Artificial Sequence
  Description of Artificial Sequence Synthetic oligonucleotide
cagatagaggctgttttttgaatcatctctggagatgg

SEQ ID NO: 142 - DNA, 19 bp, Artificial Sequence
  Description of Artificial Sequence Synthetic oligonucleotide
gagacccaagcttggtacc

SEQ ID NO: 143 - DNA, 21 bp, Artificial Sequence
  Description of Artificial Sequence Synthetic oligonucleotide
ctgactttaactcctaacatg

SEQ ID NO: 144 - DNA, 54 bp, Artificial Sequence
  Description of Artificial Sequence Synthetic oligonucleotide
aatcatgtctgcatctccaggggaaaaggtcacaatcacttgcagggccagctc

SEQ ID NO: 145 - DNA, 57 bp, Artificial Sequence
  Description of Artificial Sequence Synthetic oligonucleotide
caagtgattgtgaccttttcccctggagatgcagacatgattgctggagactgggag

SEQ ID NO: 146 - DNA, 42 bp, Artificial Sequence
  Description of Artificial Sequence Synthetic oligonucleotide
acttataccggtcagatcgttctcacccagtctccagcaatc

SEQ ID NO: 147 - DNA, 42 bp, Artificial Sequence
  Description of Artificial Sequence Synthetic oligonucleotide
accagcagaagccaggatcccagcccaaaccctggatttctg

SEQ ID NO: 148 - DNA, 28 bp, Artificial Sequence
  Description of Artificial Sequence Synthetic oligonucleotide
cagcgcaatggaggctgaagatgctgcc

SEQ ID NO: 149 - DNA, 27 bp, Artificial Sequence
  Description of Artificial Sequence Synthetic oligonucleotide
ttgggctgggatcctggcttctgctgg

SEQ ID NO: 150 - DNA, 36 bp, Artificial Sequence
  Description of Artificial Sequence Synthetic oligonucleotide
cttcagcctccattgcgctgattgtgagagagtaag

SEQ ID NO: 151 - DNA, 56 bp, Artificial Sequence
  Description of Artificial Sequence Synthetic oligonucleotide
attaccttcgaaaagtgtacttacgttttatttccaggttggtcccccctccgaac

SEQ ID NO: 152 - DNA, 56 bp, Artificial Sequence
  Description of Artificial Sequence Synthetic oligonucleotide
attaccttcgaaaagtgtacttacgttttatttccagcttggtcccccctccgaac

SEQ ID NO: 153 - protein, 80 aa, Mus musculus
QIVLTQSPAIMSASPGEKVTITCWFQQKPGTSPKLWIYGVPARFSGSGSGTSYSLTISRMEAEDAATYYCFGSGTKLELK

SEQ ID NO: 154 - protein, 80 aa, Homo sapiens
DIQMTQSPSTLSASVGDRVTITCWYQQKPGKAPKLLMYGVPSRFIGSGSGTEFTLTISSLQPDDFATYYCFGQGTKVEVK

SEQ ID NO: 155 - protein, 80 aa, Unknown Organism
  Description of Unknown Organism CEA4-8A
EIVLTQSPSSLSASVGDRVTITCWYQQKPGKAPKLLIYGVPSRFSGSGSGTDFTLTISSLQPEDFATYYCFGGGTKVDIK

SEQ ID NO: 156 - protein, 80 aa, Artificial Sequence
  Description of Artificial Sequence Illustrative FR Best Fit peptide
EIVLTQSPATLSASVGDRVTIMCWFQQKPGKAPKLLIYGVPDRFSGSGSGTSFSLTISSLQAEDVATYYCFGQGTKLELK

SEQ ID NO: 157 - protein, 87 aa, Mus musculus
QVQLQQSGAELAKPGASVKMSCKASGYTFTWVKQRPGQGLEWIGKATLTADKSSSTAYMQLSSLTFEDSAVYYCARWGQGTTLTVSS

SEQ ID NO: 158 - protein, 87 aa, Homo sapiens
QVQLVQSGAEVKKPGSSVKVSCKASGGTFSWVRQAPGQGLEWMGRVTITADESTNTAYMELSSLRSEDTAFYFCAGEYNGGLVTVSS

SEQ ID NO: 159 - protein, 87 aa, Unknown Organism
  Description of Unknown Organism A10 (045903)
QVQLQQSGAEVAKPGASVKVSCKASGDTFTWVKQRPGQGLEWIGKATLTVDKSSSTAYMQLSSLTSEDSAVYYCARWGQGTTVTVSS

SEQ ID NO: 160 - protein, 87 aa, Artificial Sequence
  Description of Artificial Sequence Illustrative FR Best Fit peptide
QVQLQQSGAEVAKPGASVKVSCKASGDTFTWVKQRPGQGLEWIGKATLTVDKSSSTAYMQLSSLTSEDSAVYYCARWGQGTTLTVSS

SEQ ID NO: 161 - protein, 80 aa, Homo sapiens
DIVMTQSHKFMSTSEGDWVSITCWYQQKPGQSPKLLIYGVPDRFSGSGSGTDFTFTISSVQAEDLAVYYCFGSGTNLEIK

SEQ ID NO: 162 - protein, 80 aa, Homo sapiens
DIVMTQSPDSLAVSLGERATITCWYQQKPGQSPKLLIYGVPDRFSGSGSGTDFTLTISSLQAEDVAVYYCFGSGTKVEIK

SEQ ID NO: 163 - protein, 80 aa, Artificial Sequence
  Description of Artificial Sequence Illustrative Framework peptide
DIVMTQSPSSLSASVGDRVTITCWYQQKPGKAPKLLIYGVPSRFSGSGSGTDFTLTISSLQPEDFATYYCFGGGTKVEIK

SEQ ID NO: 164 - protein, 80 aa, Artificial Sequence
  Description of Artificial Sequence Illustrative FR Best Fit peptide
DIVMTQSPSSLSASVGDRVSITCWYQQKPGQSPKLLIYGVPDRFSGSGSGTDFTLTISSLQAEDVAVYYCFGQGTNLEIK

SEQ ID NO: 165 - protein, 87 aa, Homo sapiens
EVQLQQSGPELVKPGASMKISCEASGYSFTWVKQSHGMNLEWIGKATLTVDKSSGTAYMELLSLTSEDSAVYFCARWGQGTSVTVSS

SEQ ID NO: 166 - protein, 87 aa, Homo sapiens
EVQLVQSGAEVKKPGASVKVSCKASGYSFTWVKQSPGMNLEWIGKATLTVDKSTSTAYMELSSLRSEDTAVYFCARWGQGTLVTVSS

SEQ ID NO: 167 - protein, 87 aa, Artificial Sequence
  Description of Artificial Sequence Illustrative Framework peptide
QVQLQQSGAELVKPGASVKLSCKASDYTFTWVKQRPGQGLEWIGKATLTVDKSSSTAYMQLSSLTSEDSAVYYCARWGQGTTVTVSS

SEQ ID NO: 168 - protein, 87 aa, Artificial Sequence
  Description of Artificial Sequence Illustrative FR Best Fit peptide
EVQLVQSGAEVKKPGASVKVSCKASGYSFTWVKQRPGQGLEWIGKATLTVDKSSSTAYMQLSSLTSEDSAVYYCARWGQGTSVTVSS

SEQ ID NO: 169 - protein, 80 aa, Artificial Sequence
  Description of Artificial Sequence Illustrative Framework peptide
DIQMTQSPASQSASLGESVTITCWYQQKPGKSPQLLIYGVPSRFSGSGSGTQFSLKINSLQPEDFGSYYCFGSGTKLEIK

SEQ ID NO: 170 - protein, 80 aa, Artificial Sequence
  Description of Artificial Sequence Illustrative FR Best Fit peptide
DIQMTQSPASQSASLGESVTITCWYLQKQGKSPQLLVYGVPSRFSGSGSGTDFSFTISSLQPEDFATYYCFGQGTKLELK

SEQ ID NO: 171 - protein, 87 aa, Artificial Sequence
  Description of Artificial Sequence Illustrative Framework peptide
QVQLQQSGAELVKPGASKLVSCKASDYTFTWVKQRPGQGLEWIGKATLTVDKSSSTAYMQLSSLTSEDSAVYYCARWGQGTTVTVSS

SEQ ID NO: 172 - protein, 87 aa, Artificial Sequence
  Description of Artificial Sequence Illustrative FR Best Fit peptide
QIQLVQSGGEVKKPGASRQVSCKASGYSFTWVRQSPGKGLEWIGKATLTVDKSSSTAYMQLSSLTSEDSAVYYCARWGQGTTLTVSS

SEQ ID NO: 173 - protein, 80 aa, Artificial Sequence
  Description of Artificial Sequence Illustrative Framework peptide
QIVLTQSPAIMSASPGDKVTITCWFQQKPGTSPKLWIYGVPARFSGSGSGTSYSLTISAMEAEDAATYYCFGSGTKLEIK

SEQ ID NO: 174 - protein, 82 aa, Artificial Sequence
  Description of Artificial Sequence Illustrative Framework peptide
EVQLVESGGGLVKPRGSLRLSCAASWVRQAPGKGLEWVGRFTISRDDSKNTLYLQMNSLKTEDTAVYYCTTWGGGTLVTVSS

(19) United States
(12) Patent Application Publication
(10) Pub. No.: US 2003/0190705 A1
(43) Pub. Date: Oct. 9, 2003

(54) Method of humanizing immune system molecules

(75) Inventors: Hing C. Wong, Fort Lauderdale, FL (US); Jeffrey R. Stinson, Brookeville, MD (US); Luis A. Mosquera, Miami, FL (US)

Correspondence Address: EDWARDS & ANGELL, LLP, P.O. BOX 9169, BOSTON, MA, 02209, US

(73) Assignee: Sunol Molecular Corporation

(21) Appl. No.: 10/230,880

(22) Filed: Aug. 29, 2002

Related U.S. Application Data
(63) Continuation-in-part of application No. 09/990,586, filed on Nov. 21, 2001.
(60) Provisional application No. 60/343,306, filed on Oct. 29, 2001.

(51) Int. Cl. C12P 21/02; C12N 5/06; C07K 16/44; C07H 21/04

(52) U.S. Cl. 435/69.1; 435/328; 435/320.1; 530/387.3; 536/23.53

(57) ABSTRACT

Disclosed are methods for making immune system molecules, particularly humanized antibodies and fragments thereof. A typical method optimizes similarity between individual antibody framework regions to help identify human framework regions suitable for making the molecules. Comparisons between larger antibody frameworks, hypervariable domains or both is avoided. Also disclosed are antibodies that have been humanized by the method. The invention has a wide spectrum of applications including use in the production of humanized monoclonal antibodies with suitable binding affinity and minimized human immunogenicity.

### CROSS-REFERENCE TO RELATED APPLICATIONS

The present application is a continuation-in-part of U.S. application Ser. No. 09/990,586 entitled Antibodies For Inhibiting Blood Coagulation and Methods of Use Thereof by Jiao, J. et al. as filed on Nov. 21, 2001, which application claims priority to U.S. Provisional Application U.S. S No. 60/343,306 as filed on Oct. 29, 2001. The U.S. application Ser. No. 09/990,586 is related to U.S. application Ser. No. 09/293,854 which application is a divisional of U.S. application Ser. No. 08/814,806 (now U.S. Pat. No. 5,986,065). The disclosures of the U.S. application Ser. Nos. 09/990,586, 60/343,306 and the U.S. Pat. No. 5,986,065 are incorporated by reference.

### FIELD OF THE INVENTION

The present invention features methods for making humanized immune system molecules. In one aspect, the invention provides methods for humanizing antibodies that involve optimizing sequence similarity between individual antibody framework regions rather than a larger framework or variable domain. The invention has a wide spectrum of applications including use in the production of humanized monoclonal antibodies with suitable binding affinity and minimized human immunogenicity.

### BACKGROUND OF THE INVENTION

There is general recognition that antibodies have important uses. For example, many are known to detect antigen with exquisite specificity. Polyclonal and monoclonal antibodies have been reported. See generally Molecular Biology of the Cell (B. Alberts et al. Eds. 2^{nd} edition) (1989) Garland Publishing, Inc. New York and references cited therein.

There has been substantial interest toward understanding antibody structure and function. For instance, much is known about the structure and function of variable (V) domains. Nearly all antibody V domains include hypervariable complementarity determining regions (“CDRs”) and framework regions (“FRs”). For most antibody molecules, a single CDR is separated from another CDR by an intervening FR. It is generally recognized that FRs serve as molecular scaffolding that helps position CDR loops in the proper configuration for antigen recognition and binding. See generally B. Alberts et al., supra, and references cited therein.

Many workers have used “framework” to describe the FRs in their entirety from a single antibody light chain variable domain (VL) or heavy chain variable domain (VH). Accordingly, one antibody V domain includes individual framework region subsets (FR1, FR2, FR3 and FR4) in which each subset is linked to its corresponding CDR (CDR1, CDR2, and CDR3). A framework consists of the framework region subsets in their entirety from a single antibody.

At least for some antibodies, the amino acid residues in FRs are thought to contibute to antigen binding. See Foote, J. and G. Winter (1992) J. Mol. Biol. 224: 487.

There have been attempts to use monoclonal antibodies as therapeutic agents. However, the approach has been challenging at least with certain antibodies. For example, some human subjects have been reported to develop undesirable immune side reactions following exposure to antibodies derived from non-human sources. These reactions, in some instances, can pose serious health problems and may be life threatening.

There have been attempts to make antibodies more immunologically acceptable to human subjects. Although there are some reports of human monoclonal antibodies, it is generally accepted that such molecules are difficult to make and use.

An alternative strategy for making antibodies more acceptable to humans has been to introduce genes that encode human antibodies into non-human animals (e.g. mice). Such “transgenic” animals have been reported to make antibodies with human sequence. However, making such animals has often been difficult and time consuming. Also, these transgenic mice are proprietary and possess less than optimal human antibody gene repertoires.

Another approach for making immunologically acceptable antibodies has been to use recombinant DNA technologies. One concept has been to clone and modify non-human antibodies so that resulting molecules resemble human antibodies. Collectively, such antibodies have been referred to as “humanized”. See U.S. Pat. No. 5,766,886 to Studnicka et al.; U.S. Pat. No. 5,693,762 to Queen et al.; U.S. Pat. No. 5,985,279 to Waldeman et al.; U.S. Pat. No. 5,225,539 to Winter; U.S. Pat. No. 5,639,641 to Pedersen, et al.; and references cited therein.

Several specific approaches have been proposed for humanizing antibodies.

One strategy involves making what has been described as a chimeric antibody molecule. Typically, an antigen is used to immunize a non-human animal such as a mouse. Monoclonal antibodies are then made from the animal using conventional techniques. Genes encoding the monoclonal antibody are generated using conventional polymerase chain reaction (PCR) amplification. Isolated sequences encoding murine antibody variable domains are genetically fused by standard recombinant DNA technology to the sequences encoding the constant domains of human antibodies. This strategy has been used to make a human-mouse chimeric antibody. See e.g., S. L. Morrison and V. Oi (1989) Adv. Immunol. 44: 65.

Unfortunately, there have been reports of problems making and using some chimeric antibodies. Specifically, there has been disclosure of unacceptable immune responses after administration of the antibodies to human subjects. Reduced circulatory half-life is also believed to be a problem with at least some chimeric antibodies. See e.g., Boulianne et al. in Nature 312: 643 (1984); Junghans et al. Cancer Res. 50: 1495 (1990) and Bruggemann et al. (1989) J. Exp. Med. 170:2153.

Additional strategies for making humanized antibodies have been reported.

One approach has been described as “CDR grafting” (sometimes called “framework grafting” or “antibody reshaping”). CDR grafting has been taught to involve inserting murine CDRs into a human V domain. The murine CDRs are then substituted for the corresponding human CDRs. See E. Padlan Mol. Immunol. 28: 489(1991). Usually, all FRs in the V domain are derived from a single human antibody. Some in the field have predicted that human FRs of the framework will correctly position the murine CDRs to bind antigen. The expectation has been that such antibodies will evade recognition by the human immune system. See, e.g., Jones et al., Nature 321: 522-525 (1986); Junghans et al., supra.

However, there have been problems using many CDR grafting techniques.

For instance, some “grafted” antibodies have unacceptable antigen binding properties. See Gorman, et al. PNAS (USA) 88: 4181 (1991). Attempts to improve antigen binding, usually by adding murine residues back to CDR grafted V domain, have not always been successful. See Queen et al., PNAS (USA) 86: 10029 (1989); and Co, et al., PNAS (USA) 88: 2869 (1991).

There have been further attempts to produce humanized antibodies using what has been described as “antibody resurfacing”. One approach has been to replace exposed amino acid residues of an allogeneic antibody with those usually found in host antibodies. The hope has been to decrease immunogenicity of the antibody while preserving as much antigen binding function as possible. See Roguska, et al. PNAS (USA) 91: 969 (1994).

Unfortunately, there have been problems using many of the prior antibody reshaping techniques.

For instance, many reshaped antibodies are thought to exhibit unacceptable antigen binding properties. See E. Padlan Mol. Immunol. 28: 489(1991). Further, most reshaping techniques require detailed knowledge about the structure of the antibody to be reshaped. Substantial information about solvent accessibility and related parameters is often required. However such information is not always available for antibodies that need to be humanized.

A major drawback shared by most antibody humanization techniques is that for a desired antibody to be humanized, a relatively large framework or V domain is selected for grafting or reshaping manipulations. The concept of using such large antibody segments as the basis for manipulating non-human antibodies has produced undesirable results e.g., reducing antigen binding affinity and retaining unacceptable immunogenicity. To address these deficiencies, additional efforts are employed to restore affinity. These efforts typically end in a compromise between adding enough non-human amino acid residues to boost affinity while not adding so much that immunogenicity is also increased.

More particularly, it is believed that many of the prior humanization techniques involve manipulations that use an unacceptably large antibody framework, or worse, entire V domain to humanize antibodies. According to one approach, a large non-human framework or V domain is used as a query to identify a set of human frameworks with acceptable sequence identity to the query. The identified human framework is often said to be the “best fit” if it exhibits the greatest sequence identify to the query non-human sequence with respect to the search parameters used.

It is believed that past practice of selecting best fit human frameworks based on comparisons with relatively large framework or V domains has hindered attempts to make and use many humanized immune system molecules. For instance, use of the prior antibody humanization methods is believed to have missed many potential “best fit” matches. That is, by limiting the pool of “best fit” matches to framework or V domains, the ability to humanize many antibodies has been compromised.

Additionally, many prior methods for making humanized antibodies have searched for “best fit” matches using complete antibody sequences. However this strategy poses significant disadvantages.

For example, such searching approaches cannot include FR sequences from incompletely sequenced human antibodies. This shortcoming has made it difficult to take advantage of more complete collections of human antibody sequence information.

The prior methods for making humanized antibodies are believed to suffer from other deficiencies.

For instance, there is recognition in the field that many humanized antibodies do not exhibit optimal antigen binding affinity. Steric hinderance (sometimes called interference) between amino acids of the non-human CDRs adjacent to the human FRs and the amino acid residues in key locations in the FRs (referred to as vernier zone residues) is thought to contribute to the problem. Because the prior humanization methods are believed to miss many potential “best fit” human FR sequences, the ability to address these and related interference problems has been limited.

It would be useful to have more effective methods for humanizing immune system molecules. More specifically, it would be useful to have methods for making humanized antibodies that involve optimizing sequence similarity between individual framework subsets as the basis for making humanized immune system molecules with suitable binding affinity and minimized human immunogenicity.

### SUMMARY OF THE INVENTION

The present invention features effective and useful methods for humanizing immune system molecules. In one aspect, the invention provides methods for making humanized antibodies and fragments thereof that optimize sequence similarity between individual framework region subsets (FRs). Comparisons between larger antibody frameworks and/or variable domains is avoided and selected “best fit” human FRs are used to make the humanized immune system molecules. The invention has a wide variety of important applications including use in making humanized immunoglobulins with suitable antigen affinity and minimized human immunogenicity.

We have discovered a novel method for “humanizing” a broad spectrum of immune system molecules e.g., antibodies and antigen-binding fragments thereof The method generally involves optimizing sequence similarity between at least one non-human FR subset and at least one corresponding human FR. Sequence similarity is typically optimized for one, two or three of the non-human FRs, more preferably for all four of the FRs (i.e., FR1, FR2, FR3 and FR4). Such sequence similarity can be optimized by considering one FR at a time (e.g., consecutive FRs like FR1 and FR2) or more than one at a time (e.g., all four FRs together) as needed to suit intended use. One or more identified human FR subsets that have the highest sequence similarity to the corresponding non-human FR subset is selected for further manipulation. The selected human FR subset is often referred to as being the “best fit” with respect to the non-human FR subset to which it has been compared. Practice of the invention further involves substituting at least one of the “best fit” human FR subsets for at least one of the corresponding non-human FR subsets. Preferred use of the invention is thus intended to maximize sequence similarity between at least one and preferably all of the individual human and non-human FR subsets. Optimizing sequence similarity at the level of framework sets (i.e., FR1, FR2, FR3 and FR4 together) and whole variable domains is avoided. Selected best fit human FR subsets are subsequently used as components for assembling the humanized immune system molecules disclosed herein.

The present invention provides many important advantages.

For example, prior practice has often detected best fit human frameworks (i.e., FR1-4) based on similarity between entire framework sets or antibody variable domains. This approach is believed to have hampered efforts to make and use humanized immunoglobulins e.g., by causing a lack of FR selection sensitivity. Moreover, many best fit matches between individual human FRs have been missed.

More specifically, it is believed that prior humanization methods have severely limited the number of best fit possibilities. In contrast, practice of the invention is not so constrained. That is, optimal use of the invention involves identifying and maximizing sequence similarity at the level of individual FR subsets. Less sensitive sequence comparisons between much larger framework sets and/or antibody variable domains are specifically avoided. These and related advantages provided by the invention expand the number of best fit possibilities, typically by the arithmetic factorial of the number of FR subsets on the immune system molecule to be humanized. This feature of the invention substantially enhances opportunities to make and use humanized molecules, helps lower costs associated with humanization procedures, and provides a rational basis for assembling nearly any humanized immune system molecule of interest.

By way of illustration, the present invention can be used to humanize a desired non-human antibody. In this example, the pool of available human FR subsets for sequence comparison is the arithmetic factorial of each of the four framework subsets (FR1, FR2, FR3 and FR4) on each of the light and heavy chains for which sequence information is available (including, significantly, FR sequence information from incomplete variable domain sequences). A key result is an increase in pool size of at least about four fold. In some embodiments, the pool size will be even larger due to availability of best fit FRs from incomplete V domain sequences. This is a significant advantage over prior methods, since it boosts the size of the pool of human FRs subsets available for selection. Potential best fit human FR possibilities are thus enhanced. Accordingly, the chances of detecting a best fit human FR subset is higher according to the invention when compared to past approaches.

Practice of the present invention provides other significant advantages.

As discussed, it is an object of the invention to optimize and select the best fit for each non-human FR subset to be humanized. This process, unlike past approaches, is conducted essentially independently of the other FRs within the subject immune system molecule. Thus, the number of mismatched amino acids, to the extent they exist at all, will be substantially lower by using the invention method. Unlike past methods which are often limited by the pool of fully sequenced human antibodies and variable domains available, use of the invention provides new opportunities for selecting best fit human FR subsets. The invention further provides better chances of detecting best fit human FRs with no or few mismatches when compared to the corresponding non-human FR. Practice of the present methods will thus enhance opportunities for making and using a wide variety of immune system molecules. Importantly, such methods can be used to prepare humanized antibodies and fragments whose antigen binding affinity is substantially preserved when compared to a parental non-human or chimeric molecule from which it was obtained.

Accordingly, and in one aspect, the invention provides a method for producing a humanized antibody variable (V) domain or fragment thereof. Preferred fragments specifically bind antigen alone or in combination with its corresponding V domain binding partner or fragment. In most embodiments, the method includes at least one and preferably all of the following steps.

Typically, at least one amino acid sequence of a desired non-human antibody FR subset, preferably less than about four of such FRs, more preferably about one FR such as FR1 or FR2, is compared to a pool of corresponding human amino acid antibody or variable domain sequences or fragments thereof. In most instances, a collection of corresponding human antibody framework amino acid sequences is used for the comparison although other collections may be used for some applications including collections of partially framework sequences. A corresponding human FR subset is selected from the pool that has the best fit with respect to the corresponding non-human FR subset. Generally, a suitable best fit human FR subset will exhibit at least about 75% sequence identity, preferably at least about 80% sequence identity, more preferably at least about 90% up to about 100% sequence identity when compared with the corresponding non-human FR subset.

Subsequently, the non-human FR subset is mutagenized to encode essentially the best fit human FR subset identified above. One or a combination of standard mutagenesis procedures can be used as discussed below. Preferably, mutagenesis of the non-human FR subset is conducted to produce a humanized FR subset (sometimes called “huFR”) that is substantially identical to the selected human FR subset i.e., at least about 75% identical, preferably at least about 80% identical, more preferably at least about 90% identical up to about 100% identical to the selected subset.

The prior humanization steps of the invention can be repeated, as needed, to humanize one or more desired FR subsets of the non-human V domain. More specifically, the steps can be repeated one, two, three, or even more times, and under conditions that produce a plurality of DNA sequences in which each DNA sequence encodes a corresponding huFR subset. In most instances (e.g., as when a fully humanized immune system molecule is required), it will be useful to repeat the steps about three to four times to humanize each of the FR subsets (FR1, FR2, FR3, and FR4). Thus in embodiments in which a non-human antibody is of interest, each of the FR subsets on each of the immunoglobulin light and heavy chains will be subjected to the aforementioned humanization steps.

Successful practice of the invention does not depend on any particular order of humanizing the non-human FRs so long as intended results are achieved.

One or a combination of the DNA sequences encoding the huFR subsets is then substituted into a suitable (first) vector using standard recombinant approaches. Preferably, the first vector encodes at least the V domain of the non-human antibody to be humanized. However in some embodiments, the first vector further encodes an immunoglobulin light or heavy chain constant domain or a fragment thereof covalently linked to the V domain.

Preferred substitution steps are generally conducted under conditions in which at least one and preferably all of the huFR DNA sequences is used as a replacement for one or more corresponding non-human FR subsets encoded by the vector. The order of FR substitution is usually not important provided the intended humanized molecule is produced. Thus for example, a non-human FR1 can be humanized first followed by non-human FR2. Alternatively, the non-human FR2 can be humanized before the non-human FR1. Preferred substitution steps are conducted according to conventional manipulations and result in each of the huFR subsets being operatively linked to one or more corresponding complimentarity determining regions (CDR).

The first vector can be employed to express one or more encoded humanized immune system molecules in a wide spectrum of suitable host cells as discussed below. Generally preferred methods are conducive to expressing the humanized antibody V domain or the fragment thereof in the host cells. Additionally preferred methods are fully compatible with approaches intended to purify the humanized molecule from cell constituents which accompany it.

The invention is fully compatible with a wide range of recombinant approaches that convert a non-human amino acid at each mismatched position on a given non-human FR subset to a desired human amino acid. An example of an acceptable recombinant approach is site-directed mutagenesis or related PCR-based method. Because the pool of potential best fit human FR subsets is greater according to the invention, there is a much higher probability of identifying a human FR subset with fewer mismatches. This feature of the invention helps reduce the number of FR mismatches that need to be considered when a particular immune system molecule is to be humanized. These and other invention advantages help reduce cost and time expenditures that are typical of many prior humanization methods.

As discussed, most prior humanization approaches have been hindered by unacceptable antigen binding of the resulting molecules. More specifically, most prior humanized antibodies exhibit too low an affinity for cognate antigen when compared to the parental molecule. Many parental chimeric antibodies have better antigen binding characteristics than the humanized version of the antibody. Without wishing to be bound to theory, it is believed that such unacceptable antigen binding can be attributed, in large part, to hindrance stemming from one or more amino acids in the FR subsets proximal to the CDRs (the vernier zone residues). There are reports that at least for some antibodies, the vernier zone residues may contribute to antigen binding by the antibody V domain.

Past attempts to minimize vernier zone hindrance problems have been largely unsuccessful or require sophisticated computer algorithms to correct and these rely heavily on computer-based molecular modeling and structural knowledge.

For example, in one approach, computer-assisted modeling software has been used to help reduce the hindrance. Such software is often complicated and difficult to use. The present invention addresses the problem by optimizing the detection and selection of best fit huFR subsets without assistance from complicated software. That is, the invention provides more best fit candidates, thereby decreasing chances for mismatches in the vernier zone and elsewhere in the immune system molecule to be humanized. Accordingly, use of the invention can significantly maintain antigen binding affinity of the parental immune system molecule. This invention advantage is especially important when a non-human antibody of interest has an unusually high affinity (Kd>10⁻⁹ M or greater) for antigen. In such situations, high affinity often places demands on maintaining the CDR in the appropriate configuration. The invention provides for substantial compliance with these demands by minimizing or eliminating potential for vernier zone incompatibilities. Past practice has used X-ray crystallographic or computer based antibody information to help address the vernier zone incompatibilities. Significantly, the invention reduces and in many instances avoids user reliance on this information.

Other aspects of the invention are discussed infra.

### BRIEF DESCRIPTION OF THE DRAWINGS

FIGS. 1A and 1B shows the nucleic acid (SEQ ID NOS: 1 and 3) and amino acid (SEQ ID NOS: 2 and 4) sequences of light chain and heavy chain variable domains of H36.D2.B7, the murine anti-tissue factor antibody, with hypervariable regions (CDRs or Complementarity Determining Regions) underlined (single underline for nucleic acid sequences and double underline for amino acid sequences).

FIG. 2 is a drawing showing a plasmid map of humanized anti-TF IgG1 antibody expression vector (pSUN-34).

FIGS. 3A-D are sequences of partially and fully humanized light chain (LC) variable domains of the anti-TF antibody (SEQ ID NO.: ______). FIG. 3A shows the sequence named “LC-09” which is representative of a fully humanized LC framework (SEQ ID NO.:______). Light chain CDR sequences of cH36 and LC-09 are shown in FIGS. 3B-D (SEQ ID NOS.: ______, respectively).

FIGS. 4A-D are drawings showing the sequences of partially and fully humanized heavy chain (HC) variable domains of the anti-TF antibody (SEQ ID NOS.: ______). FIG. 4A shows the sequence named “HC-08” which is representative of a fully humanized HC framework (SEQ ID NO.: ______). Heavy chain CDR sequences for cH36 and HC-08 are shown in FIGS. 4B-D (SEQ ID NOS.: ______, respectively).

FIGS. 5A-B are sequences showing human constant domains in the IgG1 anti-tissue factor antibody (hOAT), with FIG. 5A showing the human kappa light chain constant domain (SEQ ID NO.: ______) and FIG. 5B showing the human IgG1 heavy chain constant domain (SEQ ID NO.: ______). The figures show hOAT (IgG1) constant domain amino acid sequences.

FIGS. 6A-B are sequences showing human constant domains in the IgG4 anti-tissue factor antibody (hFAT) with FIG. 6A showing the human kappa light chain constant domain (SEQ ID NO.: ______) and FIG. 6B showing the human IgG4 heavy chain constant domain (SEQ ID NO.: ______).

FIGS. 7A and 7B shows the nucleic acid (SEQ ID NOS.: ______) and amino acid (SEQ ID NOS: ______) sequences of light chain and heavy chain variable domains of A110, the chimeric anti-LTA antibody, with hypervariable regions (CDRs or Complementarity Determining Regions) underlined (single underline for nucleic acid sequences and double underline for amino acid sequences).

FIG. 8 is a drawing showing a plasmid map of an expression vector encoding humanized anti-LTA IgG1 (PJRS 391).

FIGS. 9A-H are drawings showing sequences of partially and fully humanized variable domains of the anti-LTA antibody. FIG. 9A shows the sequence of the humanized light chain (LC) variable domain framework regions (SEQ ID NOS.: ______). FIGS. 9B-D show light chain CDRs1-3 (SEQ ID NOS.: ______). FIG. 9E shows the sequences of partially and fully humanized heavy chain (HC) variable domain framework regions (SEQ ID NOS.: ______). FIGS. 9F-K show heavy chain CDRs1-3 (SEQ ID NOS.: ______).

FIG. 10 is a table showing plasmid constructs producing humanized Al 10 antibody for evaluation.

FIG. 11 is a graph showing determination of antibody expression by humanized anti-LTA by different plasmids.

FIG. 12 is a graph showing determination of LTA binding.

### DETAILED DESCRIPTION OF THE INVENTION

As discussed, the invention features methods for making humanized immune system molecules. Examples of such molecules include humanized antibody variable (V) domains, humanized antibodies (chimeric and monoclonal) as well as antigen-binding fragments thereof. For instance, the invention provides new methods for humanizing antibodies that involves optimizing sequence similarity between at least one non-human framework (FR) subset, typically two, three or four of such subsets, and one or more corresponding human FR subsets. At least one selected humanized FR subset (huFR) is substituted for the corresponding non-human FR subset to humanize the molecule. Preferred humanized immune system molecules of the invention feature good antigen binding affinity and minimal immunogenicity in human subjects.

The advantages of using the present invention to humanize immune system molecules is demonstrated throughout the disclosure including Examples 6-9, below. As discussed, a key difference between past humanization attempts and the present invention is that the invention methods searches for FRs with the best fit. In contrast, the prior attempts have used entire frameworks (usually derived from a single antibody variable domain) or worse, entire variable domains to determine best fits. Thus with the present methods identified FRS can be derived from as many as four different antibodies for each antibody chain, or a total of eight FRs per antibody. This feature of the invention greatly expands the available pool of detectable best fit FRs. This flexibility in searching for the FRs with the best fit results in fewer constraints being placed on the search so that more FRs can be considered. These benefits help provide for better fits requiring fewer amino acid substitutions, fewer changes to vernier residues and better homology or identity scores. In searching for an entire framework that has the best fit, the result is a compromise best fit over that entire framework. In some examples, the entire frameworks for both chains are taken from a single antibody, or in a less constrained search, the frameworks for each chain can come from different antibodies.

More particularly, Example 6, shown below, provides an in silico comparison between a past attempt to humanize the anti-TAC antibody and the present invention methods (sometimes referred to herein as “FR Best Fit Humanization”). As shown in this Example, use of the present invention provides a superior humanization result. That is, virtual humanization of the anti-TAC antibody in accord with the present invention produces a better antibody.

Further advantages of using the invention are apparent from the in silico results shown in Example 7 (Mc3 antibody); Example 8 (anti-TF antibody); and Example 9 (anti-LTA antibody). In these Examples including Example 6, virtual humanization of described antibodies produces a superior humanized antibody when contrasted with prior humanization approaches. Thus, the invention is one of general application that can be used to make and use a wide range of humanized immune system molecules.

As discussed, the invention can be used to humanize a wide spectrum of immune system molecules such as antibodies and fragments thereof. Particular invention methods can be used to produce a humanized antibody variable (V) domain or antigen binding fragment thereof. As mentioned, preferred fragments specifically bind antigen typically in combination with a corresponding V domain binding partner or fragment. In one embodiment, the method includes at least one and preferably all of the following steps:

a) comparing the amino acid sequence of a framework region (FR) subset of a non-human antibody variable (V) domain to a collection of human antibody frameworks or variable domain amino acid sequences or sequences of fragments thereof,

b) selecting a human FR subset from the collection having the greatest amino acid sequence identity to the non-human FR subset,

c) mutagenizing DNA of the non-human FR subset to encode a humanized FR subset having an amino acid sequence substantially identical to the selected human FR subset from step b),

d) repeating steps a) thru c) for each of the FR subsets in the non-human V domain to produce a plurality of DNA sequences in which each DNA sequence encodes a humanized FR subset (huFR); and

e) substituting into a first vector encoding at least the V domain of the non-human antibody, each of the huFR DNA sequences from step d) for the corresponding non-human FR subsets encoded by the vector; wherein the substitution operatively links each of the huFRs to its corresponding complimentarity determining region (CDR); and expressing the first vector in host cells and under conditions conducive to making the humanized antibody V domain, fill antibody or the antigen binding fragment thereof

The V domain or antigen binding fragment to be humanized will include at least one murine complimentarily determining region (CDR). As will be appreciated, immunoglobulin light and heavy chain share certain structural similarities e.g., each includes a framework of four framework region subsets (FR1-4) whose sequences are relatively conserved. Each of FR1-4 (FR1, FR2, FR3, FR4) are covalently connected by three CDRs i.e., CDR1, CDR2, CDR3. There is general recognition that the four FRs largely adopt a beta-sheet configuration and the interconnected CDRs form connecting loops, and in some instances, forming part of the beta-sheet structure. Most CDRs are held close to adjoining FRs, and with a corresponding CDR from the opposite light or heavy chain, help form the antigen binding site. A wide range of CDRs and FRs have been disclosed. See e.g., Kabat et al. in Sequences of Proteins of Immunological Interest Fifth Edition, U.S. Dept. of Health and Human Services, U.S. Government Printing Office (1991) NIH Publication No. 91-3242.

See also EP-A-0239400 and U.S. Pat. No. 5,985,279 (describing methods of making altered antibodies in which CDRs are derived from different species than the FR).

By the phrase “antigen binding fragment” is meant at least a part of an antibody that specifically binds antigen. An example of such a fragment includes an antibody V domain and its V domain binding partner. Further suitable fragments further include parts of the V domain having a combined molecular mass for the V domain and it V domain binding partner of between from about 15 kilodaltons to about 40 kilodaltons, preferably between from about 20 kilodaltons to about 30 kilodaltons, more preferably about 25 kilodaltons as determined by a variety of standard methods including SDS polyacrylamide gel electrophoresis or size exclusion chromatography using appropriately sized marker fragments, mass spectroscopy or amino acid sequence analysis.

Additionally suitable antigen binding fragments include at least part of an antigen binding V domains alone or in combination with a cognate constant (C) domain or fragment thereof (“cognate” is used to denote relationship between two components of the same immunoglobulin heavy (H) or light (L) chain). Typical C domain fragments have a molecular mass of between from about 5 kilodaltons to about 50 kilodaltons, more preferably between from about 10 kilodaltons to about 40 kilodaltons, as determined by a variety of standard methods including SDS polyacrylamide gel electrophoresis or size exclusion chromatography using appropriately sized marker fragments, mass spectroscopy or amino acid sequence analysis. Additionally suitable antigen binding fragments are disclosed below.

By the term, “specific binding” or a similar term is meant a molecule disclosed herein which binds another molecule, thereby forming a specific binding pair. However, the molecule does not recognize or bind to other molecules as determined by, e.g., Western blotting ELISA, RIA, mobility shift assay, enzyme-immunoassay, competitive assays, saturation assays or other protein binding assays know in the art. See generally, Sambrook et al. in Molecular Cloning: A Laboratory Manual (2d ed. 1989); and Ausubel et al., Current Protocols in Molecular Biology, John Wiley & Sons, New York, 1989. See Harlow and Lane in, Antibodies: A Laboratory Manual (1988) Cold Spring Harbor, N.Y. for examples of methods for detecting specific binding between molecules.

By the phrase “humanized” is meant an immunoglobulin that includes at least one human FR subset, preferably at least two or three of same, more preferably four human FR subsets, and one or more CDRs from a non-human source, usually rodent such as a rat or mouse immunoglobulin. Typically preferred humanized immunoglobulins of the invention will include two or more preferably three CDRs. Constant domains need not be present but are often useful in assisting function of humanized antibodies intended for in vivo use. Preferred constant domains, if present, are substantially identical to human immunoglobulin constant domains i.e., at least about 90% identical with regard to the amino acid sequence, preferably at least about 95% identical or greater. Accordingly, nearly all parts of the humanized immunoglobulin, with the possible exception of the CDRs, are preferably substantially identical to corresponding parts of naturally occurring human immunoglobulin sequences.

Methods for determining amino acid sequence identity are standard in the field and include visual inspection as well as computer-assisted approaches using BLAST and FASTA (available from the National Library of Medicine (USA) website). Preferred matching programs for most embodiments are available from website for the international ImMunoGeneTics (IMGT) database and a more preferred matching program for this embodiment is the program called Match which is available in the Kabat database. See Johnson G, Wu T. “Kabat database and its application: Future directions.” Nucleic Acids Res. (2001) 29:205-206.

By the phrase “humanized antibody” is meant an antibody that includes a humanized light chain and a humanized heavy chain immunoglobulin. See S. L. Morrison, supra; Oi et al., supra; Teng et al., supra; Kozbor et al., supra; Olsson et al., supra; and other references cited previously. Accordingly, a “humanized antibody fragment” means a part of that antibody, preferably a part that binds antigen specifically.

As discussed, the invention includes one or more method steps intended to compare and optimize the amino acid sequence of each individual non-human FR to a collection of human amino acid sequences, preferably a collection of sequences which includes human antibody framework amino acid sequences. In practice, the FR in the human framework with the highest sequence identity score has been an FR corresponding to the non-human antibody FR, but this is not necessarily required by the search parameters. By “corresponding” is meant relationship between two FRs from the same or similar position on the antibody V domain. For instance, a rodent FR1 from a given antibody light chain corresponds to a human FR1 from that light chain. Correspondence is often denoted by FR number i.e., a rodent FR1 corresponds with human FR1, a rodent FR2 corresponds with human FR2, etc.

In one embodiment of the method, the described V domain or fragment is from a non-human antibody light chain. As discussed the precise order of FR subset humanization is typically not important. Thus in one example of the invention, the light FR subset of step a) in the method will be the first variable domain framework region (FR1). However in other embodiments, other FR subsets will be humanized before FR1 e.g., FR2, FR3 or FR4.

As discussed, step b) of the method involves selecting a human FR subset from a plurality of human amino acid sequences having the greatest amino acid sequence identity to the non-human FR subset. In embodiments in which the non-human framework to be humanized is FR1, the sequence identity between the FR1 of the non-human antibody light chain and the selected human FR subset is preferably at least about 70%, more preferably at least about 80%, even more preferably at least about 95%.

As also discussed, particular invention methods involve iterative (typically sequential) humanization of each non-human framework region. Thus in embodiments in which FR1 is manipulated first, the method subsequently includes manipulation of FR2, FR3 and FR4. As mentioned, the precise order of humanizing the FRs is not important but in the interest of convenience, it may be helpful to humanize the light and heavy frameworks in numerical order i.e., FR1, FR2, FR3 and FR4.

Thus in one invention embodiment, step d) of the method will further include comparing the second non-human framework region (FR2) of the non-human light chain (or heavy chain) V domain to the collection and selecting a human FR subset having at least about 70% sequence identity, preferably at least about 80%, more preferably at least about 95% sequence identity to its human FR subset (in practice, this is typically a human FR2). The step d) of the method will further include comparing a third framework region (FR3) of the non-human light chain (or heavy chain) V domain to the collection and selecting a human framework region having at least about 70% sequence identity, preferably at least about 80%, more preferably at least about 95% sequence identity to a human FR subset (in practice, this is typically a human FR3). Typically, the step d) will further include comparing a fourth framework region (FR4) of the non-human light chain (or heavy chain) V domain to the collection and selecting a human framework region having at least about 70% sequence identity, preferably at least about 80%, more preferably at least about 95% sequence identity to its corresponding human framework subset (in practice, this is typically a human FR4).

As will be apparent from the present disclosure, the invention can be employed to humanize a wide variety of immune system molecules including light chain V domains, heavy chain V domains of any heterodimeric immunoglobulin-like molecules, including but not limited to T-cell receptors, major histocompatibility complexes, antibodies; and antigen binding fragments thereof. In one embodiment, the humanized light chain (or heavy chain) includes the following components covalently linked in sequence: huFR1-CDR1-huFR2-CDR2-huFR3-CDR3-huFR4. Also encompassed by the invention method are antigen-binding fragments of the light or heavy chain V domains as well as molecules that further include relevant constant domains and fragments thereof.

As mentioned, it is an object of the invention to produce humanized antibodies (and antigen binding fragments) in which potential vernier zone hindrance problems on at least one of the V domains is minimized and preferably eliminated. This invention feature helps to maximize specific binding between the immune system molecule and its cognate antigen. Thus in one embodiment of the method, vernier zone amino acid residues in each FR subset on at least one of the light and heavy chains of the variable domain is identical when the non-human FR subset is compared with the corresponding human FR subset of the antibody V domain. For example, the vernier zone amino acid residues of non-human FR1 on the light or heavy chain should be identical to corresponding residues in the human FR1 subset.

The first vector used in the method typically includes sequence information needed for suitable expression of the encoded immune system molecule in a desired host. In embodiments in which the immune system molecule is a humanized light chain V domain, it will often be useful if the first vector further includes a human light chain constant domain or fragment thereof. Typically, but not exclusively, the human light chain constant domain or fragment will be covalently linked to the humanized light chain.

In preferred embodiments, the human light chain constant domain is C_{κ}, C_{λ} or a fragment thereof. Often, the humanized light chain fragment will have an amino acid length of between from about 80 to about 250 amino acids, preferably between from about 95 to about 235 amino acids, more preferably between from about 104 to about 225 amino acids. The size of the humanized light chain fragment can be determined by a variety of standard methods including SDS polyacrylamide gel electrophoresis or size exclusion chromatography using appropriately sized marker fragments, mass spectroscopy or amino acid sequence analysis.

In embodiments in which the humanized immune system molecule is a heavy chain V domain, the humanized heavy chain fragment will have an amino acid length of between about 80 to about 650 amino acids, preferably between from about 95 to about 540 amino acids, more preferably about 102 to about 527 amino acids as determined e.g., by standard SDS polyacrylamide gel electrophoresis or size exclusion chromatography using appropriately sized marker fragments, mass spectroscopy or amino acid sequence analysis.

As discussed, it is an object of the invention to provide novel humanization methods in which each framework region from a non-human immune system molecule is independently compared to a collection of human framework subsets. So for example, to humanize a chimeric antibody, the sequence of the first framework region (FR1) in the heavy chain (HC) variable domain is compared to all known sequences for FR subsets in the heavy chain variable domains of human antibodies. Candidates with the highest degree of identity or homology (fewest number of mismatches in the amino acid sequence) are identified. The process is then preferably repeated for each of FR2, FR3 and FR4 for the HC. A similar process is performed for the FR in the variable domain of the light chain. Best fit human FR subsets may be taken from the same or different antibodies as needed to suit an intended use of the invention. This is a significant departure from other humanization methods in which the best fit selected is a single framework in its entirety from a single human antibody sequence.

In a particular embodiment of the method, the first vector further includes a human heavy chain constant domain or fragment thereof covalently linked to the humanized heavy chain. Preferably, the human constant domain is one of an IgG1, IgG2, IgG3 or IgG4 isotype or a fragment thereof.

By the phrase “chimeric antibody” or related phrase including plural forms is meant antibodies whose light and heavy chain genes have been constructed, typically by genetic engineering, from immunoglobulin gene segments belonging to different species. For example, the variable (V) domains of the genes from a mouse monoclonal antibody may be joined to human constant (C) domains, such as γ₁, γ₂, γ₃, or γ₄. A typical therapeutic chimeric antibody is thus a hybrid protein consisting of the V or antigen-binding domain from a mouse antibody and the C or effector domain from a human antibody, although other mammalian species may be used. Specific chimeric antibodies are the anti-tissue factor antibody, cH36 and the anti-lipotechoic acid antibody, c96-110 (sometimes referred to as A110) disclosed below.

As discussed, the invention method compares the amino acid sequence of a non-human antibody variable (V) domain framework region (FR) to a plurality of human amino acid sequences, preferably a collection of human antibody framework amino acid sequences or sequences of fragments thereof. An example of such a collection is one from a database that includes a list of fully sequenced human antibodies. Unlike prior humanization methods, the collection may further include one or more amino acid sequences of partially sequenced human antibodies. Alternatively, the collection may consist essentially of only the partially sequenced human antibodies. Examples of such collections include, but are not limited to, the following databases: GenBank, IMGT, Swiss-Prot, and Kabat, supra.

In a more particular example of the invention, there is provided a method for making a humanized antibody or an antigen-binding fragment thereof. In one embodiment, the method includes at least one and preferably all of the following steps:

a) comparing the amino acid sequence of a non-human antibody light chain variable (V) domain framework region (1-FR) to a collection of human antibody light chain framework amino acid sequences,

b) selecting a human FR subset from the collection having the greatest amino acid sequence identity to the 1-FR,

c) mutagenizing DNA of the 1-FR to encode a light chain humanized FR subset (L-huFR) having an amino acid sequence substantially identical to the selected human FR subset from step b),

d) repeating steps a) thru c) for each of the FR subsets in the light chain V region to produce a plurality of DNA sequences in which each DNA sequence encodes an L-huFR,

e) substituting into a first vector encoding at least the light chain V domain of the non-human antibody, each of the L-huFR DNA sequences from step d) for the corresponding l-FRs encoded by the vector; wherein the substitution operatively links each of the L-huFRs to a corresponding complimentarity determining region (CDR),

f) comparing the amino acid sequence of a non-human antibody heavy chain variable (V) domain framework region (h-FR) to a collection of human antibody heavy chain amino acid sequences,

g) selecting a human FR subset from the collection having the greatest amino acid sequence identity to the h-FR,

h) mutagenizing DNA of the h-FR to encode a humanized heavy chain FR subset (H-huFR) having an amino acid sequence substantially identical to the selected human FR from step g),

i) repeating steps f) thru h) for each of the h-FRs in the non-human heavy chain V domain to produce a plurality of DNA sequences in which each DNA sequence encodes a H-huFR,

j) substituting into a second vector encoding at least the heavy chain V domain of the non-human antibody, each of the H-huFR DNA sequences from step i) for the corresponding h-FRs of the antibody; wherein the substitution operatively links each of the H-huFRs to a corresponding heavy chain CDR; and

k) expressing the first and second vectors in host cells and under conditions conducive to producing humanized light and heavy chains and making the humanized antibody or the fragment thereof.

In one embodiment of the foregoing method, the first and second vectors are co-expressed in the same host cell. In another embodiment, DNA molecules encoding the humanized light and heavy chains or fragments thereof are contained on a single vector and co-expressed in the same host.

Suitable first vectors for use with the foregoing antibody humanization method will include sequence information needed for suitable expression of the encoded immune system molecule. For example, acceptable first vectors will include a human light chain constant domain or fragment thereof covalently linked to the humanized light chain V domain. Preferably, the constant domain is C_{κ}, C_{λ} or a fragment thereof.

Preferred second vectors in accord with the method typically further include a human heavy chain constant domain or fragment thereof covalently linked to the humanized heavy chain V domain. For instance, the human constant domain can be one of an IgG1, IgG2, IgG3 or IgG4 isotype including fragments thereof, or other isotypes (IgA, IgD, IgE or IgM).

The present invention is also compatible with additional steps intended to purify the humanized immune system molecules from cell components that naturally accompany it. Thus in one embodiment, the forgoing methods will further include one or more steps that include purifying the humanized antibody from the host cells to produce a substantially pure preparation of the antibody. Preferably, the substantially purified humanized antibody specifically binds antigen with an affinity not less than about 10-fold lower than the parental non-human antibody.

As will be apparent, the present invention can be used to make a broad spectrum of humanized immune system molecules. For example, a humanized antibody of the invention includes: 1) light and heavy chain framework regions (FRs) that are each individually at least about 90% identical in amino acid sequence to a human FR subset, preferably at least 95% identical to same, more preferably at least about 98% up to 100% identical to the human FR subset, 2) at least one CDR from a rodent such as a mouse, preferably all the CDRs from the mouse, 3) and an immunoglobulin constant domain that is at least about 90% identical, preferably at least 95% up to about 100% identical to a corresponding human immunoglobulin constant domain. It will be appreciated that an antibody has been “humanized” by the process of “humanization” because the resultant humanized antibody is expected to bind to the same antigen as the antibody that provides the CDRs.

It will be further appreciated that particular humanized immune system molecules disclosed herein, usually humanized antibodies, may have one or more additional conservative amino acid substitutions which can be contiguous or non-contiguous as needed. For example, such substitutions will typically have substantially little or no effect on antigen binding or other immunoglobulin functions. By the phrase “conservative substitution” including plural forms is meant combinations of: gly⇄ala; val⇄ile⇄leu; asp⇄glu; asn⇄gln; ser⇄thr, lys⇄arg; and phe⇄tyr.

Antibodies of the invention are preferably substantially pure when used in the disclosed methods and assays. References to an antibody being “substantially pure” mean an antibody or protein that has been separated from components which naturally accompany it. For example, by using standard immunoaffinity or protein A affinity purification techniques, an antibody of the invention can be purified from hybridoma or cell culture medium by using native TF as an antigen or protein A resin. Similarly, native TF can be obtained in substantially pure form by using an antibody of the invention with standard immunoaffinity purification techniques. Particularly, an antibody or protein is substantially pure when at least 50% of the total protein (weight % of total protein in a given sample) is an antibody or protein of the invention. Preferably the antibody or protein is at least 60 weight % of the total protein, more preferably at least 75 weight %, even more preferably at least 90 weight %, and most preferably at least 98 weight % of the total material. Purity can be readily assayed by known methods such as SDS polyacrylamide gel electrophoresis (PAGE), column chromatography (e.g., affinity chromatography, size exclusion chromatography), mass spectroscopy or HPLC analysis.

Such substantially purified and humanized antibodies can be used to specifically bind a wide range of antigens. For instance, in one embodiment, the antibodies produced by the present methods can be used to specifically recognize and bind lipotechoic acid or a related fatty acid. Other humanized antibodies and fragments are produced that specifically recognize and bind human tissue factor.

Humanized immune system molecules according to the invention provide a broad spectrum of important uses. For instance, the humanized antibodies and antigen-binding fragments of the invention can be used to prevent or treat diseases in humans or animals. Other contemplated uses include use as a diagnostic product.

Antibodies for humanization in accord with this invention can be readily obtained from a variety of sources. Alternatively, they can be made de novo. In one approach, such molecules can be prepared by immunizing a mammal with a purified sample of native human TF, or an immunogenic peptide as discussed above, alone or complexed with a carrier or as a mixture with an adjuvant. Suitable mammals include typical laboratory animals such as sheep, goats, rabbits, guinea pigs, rats and mice. Rats and mice, especially mice, are preferred for obtaining monoclonal antibodies. The antigen can be administered to the mammal by any of a number of suitable routes such as subcutaneous, intraperitoneal, intravenous, intramuscular or intracutaneous injection. The optimal immunizing interval, immunizing dose, etc. can vary within relatively wide ranges and can be determined empirically based on this disclosure. Typical procedures involve injection of the antigen several times over a number of months. Antibodies are collected from serum of the immunized animal by standard techniques and screened to find antibodies specific for desired antigen. Monoclonal antibodies can be produced in cells which produce antibodies and those cells used to generate monoclonal antibodies by using standard fusion techniques for forming hybridoma cells. See G. Kohler, et al., Nature 256: 456 (1975). Typically this involves fusing an antibody-producing cell with an immortal cell line such as a myeloma cell to produce the hybrid cell. Alternatively, monoclonal antibodies can be produced from cells by the method of Huse, et al., Science, 256:1275 (1989). Such an antibody can be sequenced by conventional methodologies if desired.

For some applications, it may be desirable to use, as the antibody to be humanized, a chimeric antibody, e.g. antibody molecules that combine a non-human animal variable domain and a human constant domain. A variety of types of such chimeric antibodies can be prepared, including e.g. by producing human variable domain chimeras, in which parts of the variable domains, especially conserved regions of the antigen-binding domain, are of human origin and only the hypervariable regions are of non-human origin. See also discussions of humanized chimeric antibodies and methods of producing same in S. L. Morrison, Science, 229:1202-1207 (1985); Oi et al., BioTechniques 4: 214 (1986); Teng et al., Proc. Natl. Acad. Sci. U.S.A., 80:7308-7312 (1983); Kozobor et al. Immunology Today 4: 72-79 (,1983); Olsson et al. Meth. Enzymol. ⁹2: 3-16 (1983).

Additionally, transgenic mice can be employed to make particular human monoclonal antibodies. For example, transgenic mice carrying human antibody repertoires have been created which can be immunized with an antigen of interest. Splenocytes from such immunized transgenic mice can then be used to create hybridomas that secrete human monoclonal antibodies that specifically react with the antigen. See N. Loriberg et al., Nature, 368:856-859 (1994); L. L. Green et al., Nature Genet., 7:13-21 (1994); S. L. Morrison, et al., Proc. Natl. Acad. Sci. U.S.A., 81:6851-6855 (1984).

Nucleic acids which code for the antibodies of the invention also can be prepared by polymerase chain reaction (see primers disclosed in Example 1 which follows). See generally, Sambrook et al., Molecular Cloning (2^{nd} ed. 1989). Such nucleic acids also can be synthesized by known methods, e.g. the phosphate triester method (see Oligonucleotide Synthesis, IRL Press (M. J. Gait, ed., 1984)), or by using a commercially available automated oligonucleotide synthesizer. Such a prepared nucleic acid of the invention can be employed to express an antibody of the invention by known techniques. For example, a nucleic acid coding for an antibody of the invention can be incorporated into a suitable vector by known methods such as by use of restriction enzymes to make cuts in the vector for insertion of the construct followed by ligation. The vector containing the inserted nucleic acid sequence, suitably operably linked to a promoter sequence, is then introduced into host cells for expression. See, generally, Sambrook et al., supra. Selection of suitable vectors can be made empirically based on factors relating to the cloning protocol. For example, the vector should be compatible with, and have the proper replicon for the host cell that is employed. Further, the vector must be able to accommodate the inserted nucleic acid sequence. Suitable host cells will include a wide variety of prokaryotic or eukaryotic cells, such as E. coli, Bacillus subtilis, Streptomyces lividans or other bacterial hosts, Saccharomyces cerevisiae or other yeast, Aspergillus niger or other fungi, or other microbial hosts, CHO, BK or NSO mammalian cells, avian or plant cells and the like.

According to a particular invention embodiment, the method described herein will include the step of introducing one or more desired vector types into plant cells under conditions suited to expressing the vector(s) in those cells. A particular plant cell of interest is Arabidopsis. See U.S. Pat No. 6,417,429 and references cited therein.

For instance, in the foregoing method for making a V domain or antigen binding fragment thereof, step e) will include introducing the first vector into plant cells, preferably Arabidopsis, and expressing the first vector therein to produce the antibody V domain. The method is readily adapted to express the full (entire) antibody including antigen binding fragments thereof.

With respect to the previously described method for making a humanized antibody or antigen-binding fragment thereof, step k) will include introducing the first and second vectors into plant cells, preferably Arabidopsis, and expressing the vectors therein to produce the desired molecules. In some invention embodiments, what is referred to below as a “mega” vector can be used instead of first and second vectors.

The molecular weight of the antibodies of the invention will vary depending on several factors such as the intended use and whether the antibody includes a conjugated or recombinantly fused toxin, pharmaceutical, radioisotope or detectable label or the like. Also the molecular weight will vary depending on nature and extent of post-translational modifications if any (such as glycosylation) to the antibody. The modifications are a function of the host used for expression with E. coli producing non-glycosylated antibodies and eucaryotic hosts, such as mammalian cells, producing glycosylated antibodies. In general, an antibody of the invention will have a molecular weight of between approximately 20 to 150 kDa. Such molecular weights can be readily determined by molecular sizing methods such as SDS-PAGE followed by protein staining or Western blot analysis.

“Antibody of the invention” or other similar term refers to whole immunoglobulin as well as immunologically active fragments which bind a desired antigen. The immunoglobulins and immunologically active (antigen-binding) fragments thereof include an epitope-binding site (i.e., a site or epitope capable of being specifically bound by an antibody recognizing antigen). Exemplary antibody fragments include, for example, Fab, F(v), Fab′, F(ab′)₂ fragments, “half molecules” derived by reducing the disulfide bonds of immunoglobulins, single chain immunoglobulins, or other suitable antigen binding fragments (see e.g., Bird et al., Science, 242: 423-426 (1988); Huston et al., PNAS, (USA), 85:5879 (1988); Webber et al., Mol. Immunol., 32:249 (1995)). The antibody or immunologically active fragment thereof may be of animal (e.g., a rodent such as a mouse or a rat), or chimeric form (see Morrison et al., PNAS, 81:6851 (1984); Jones et al., Nature, 321: 522 (1986)). Single chain and humanized antibodies of the invention may be useful for some applications of the invention.

Thus according to another embodiment, the forgoing methods further include additional steps intended to make a humanized single-chain antibody (sc-Fv) from the humanized V regions. Additionally, fragments of humanized antibodies can be made by conventional methods, particularly F(v), F(ab′)₂, Fab′ or Fab; as well as antigen-binding fragments thereof.

Similarly, a “nucleic acid of the invention” refers to a nucleotide sequence which can be expressed to provide an antibody of the invention as such term is specified to mean immediately above.

In some instances, it may be desirable to modify the antibody of the present invention to impart a desirable biological, chemical or physical property thereto. Thus the present invention method is compatible with additional conventional steps intended to conjugate (i.e. covalently link) one or more humanized immune system molecules to a pharmaceutical agent. Such linkage can be accomplished by several methods including use of a linking molecule such as a heterobifunctional protein cross-linking agent, e.g. SPDP, carbodimide, or the like, or by recombinant methods. Particular conjugation strategies compatible with use of the humanized antibodies of this invention have been disclosed in PCT Application WO 99/21572 to Rhode, P. et al. See also Ausubel et al., Current Protocols in Molecular Biology, John Wiley & Sons, New York, (1989); Harlow and Lane in Antibodies: A Laboratory Manual, CSH Publications, NY (1988).

Particular humanized antibodies of the present invention can be polyclonal or monoclonal, as needed, and may have, without limitation, an IgG1, IgG2, IgG3 or IgG4 isotype or IgA, IgD, IgE, IgM.

The humanized antibodies disclosed herein can be produced by one or a combination of strategies including those described below in Examples 1-9.

In an approach described in Examples 1 and 4, four general steps were employed to humanize an antibody. First, the amino acid sequences of the mouse antibody light and heavy chains were obtained from the cH36 mouse-human chimeric antibody. Second, the cH36 antibody was humanized by determining which human antibody framework region gave the “best fit” i.e., most closely resembled the mouse framework region amino acid sequence. Third, relevant light and heavy chain FR sequences were humanized, and fourth, transfection and expression of isolated nucleic acid(s) that encode the humanized light or heavy chain (or humanized light and heavy chain e.g., see the mega vectors described below).

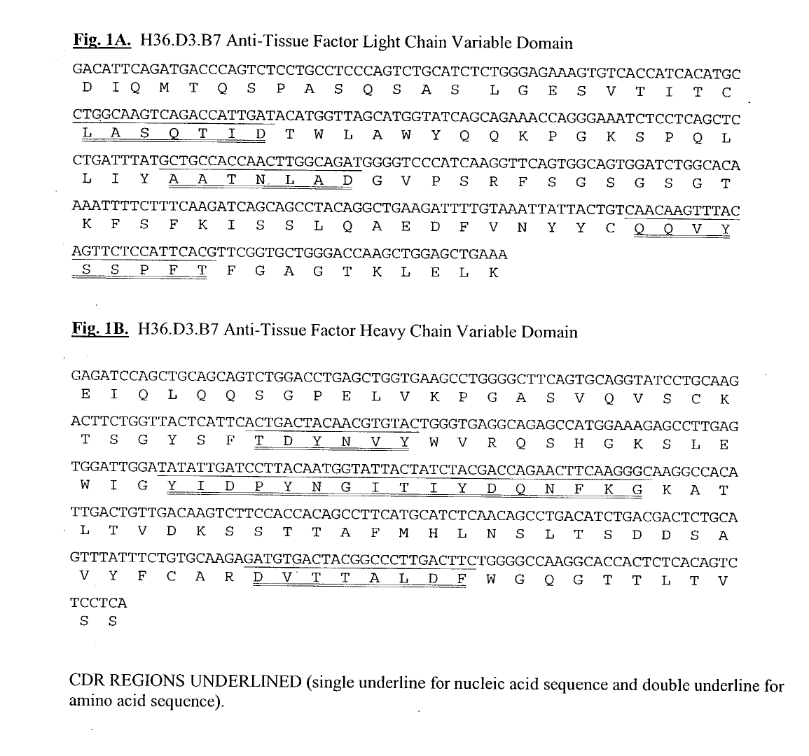

More particularly, the “FR best fit” approach was applied to humanizing the chimeric anti-tissue factor antibody cH36. In this approach, the murine light and heavy chain variable domain sequences shown in FIGS. 1A and 1B (SEQ ID NOS.: 2 and 4) were used to search (“compare”) all available protein databases for those human antibody variable domain sequences that are most homologous to the murine variable domain. See e.g., Kabat et al., supra. A number of readily available computer programs can be used to perform this step such as BLAST, FASTA and related programs. Framework regions 1, 2, 3, and 4 of the light and heavy chain were of special interest since these sites are almost universally understood to hold the CDRs in proper orientation for antigen binding. Output stemming from the search was typically a list of sequences most homologous to the query mouse sequences, the percent homology to each sequence, and an alignment of each human sequence to the corresponding murine sequence. The analysis was generally performed on the light and heavy chains independently.

According to the “FR best fit” approach, the number of mismatched amino acids was minimized between the query mouse FR subset and the human FR subset. In most cases, suitable human framework region subsets were selected based on the following identity criteria. On the light chain, the amino acid sequence of the murine FR1 was at least about 80% identical to the human FR subset; the murine FR2 was at least about 90% identical to the human FR subset, the murine FR3 was at least about 90% identical to the human FR subset; and the murine FR4 was at least about 75% identical to the human FR subset. And on the heavy chain, the amino acid sequence of the murine FR1 was chosen to be at least about 80% identical to the human FR subset; the murine FR2 was at least about 85% identical to the human FR subset; the murine FR3 was chosen to be at least about 70% identical to the human FR subset; and the murine FR4 was at least about 90% identical to the human FR subset. Typically, conservative amino acid substitutions were favored when evaluating similar candidate human framework region sequences. It was found that when such factors were considered the resulting human framework regions served as a good reference point for humanization of the chimeric cH36 antibody.

Once a decision on a desired human framework region was made, recombinant polymerase chain reaction (PCR) techniques were used to make desired amino acid substitutions in both the light and heavy chains. Typically, oligonucleotides were made and used to mutagenize mouse variable domain frameworks to contain desired residues. Oligonucleotides having a variety of lengths were employed. See WO 92/07075 for general disclosure relating to recombinant PCR and related methods.

In general, regular PCR was used for cloning, to introduce cloning or diagnostic restriction endonuclease sites, and to change amino acid residues located at the ends of the variable domains. PCR-based mutagenesis was used to change multiple amino acid residues at a time, especially when these residues were in the middle of the variable domains. Site-directed mutagenesis was used to introduce one or two amino acid substitutions at a time. After each step, the partially humanized clones were sequenced and some of these variable domains were later cloned into expression vectors. More specific methods for performing these manipulations are described in the Examples section.

After performing the foregoing “FR best fit” approach to humanizing each non-human FR, mutagenized nucleic acids encoding humanized framework region (huFR) and/or CDR were linked to an appropriate DNA encoding a light or heavy chain constant domains. Such constructs were then cloned into an expression vector, and transfected into host cells, preferably mammalian cells. These steps were achieved by using recombinant and cell culture techniques known in the field.

In one approach, a humanized antibody can be prepared by the following general method:

(a) preparing a first expression vector including a replicon appropriate for the expression host and a suitable promoter operably linked to a DNA sequence which encodes at least a variable domain of an Ig heavy or light chain, the variable domain comprising individually humanized framework regions (FR1-4) and murine CDRs 1-3 from a desired antibody, (b) preparing a second replicable expression vector including a suitable promoter operably linked to a DNA sequence which encodes at least the variable domain of a complementary Ig light or heavy chain respectively, that variable domain comprising corresponding and individually humanized framework regions (FR1-4) and murine CDRs 1-3 from the antibody;

(c) transfecting a cell line with the first or both prepared vectors; and

(d) culturing said transfected cell line to produce said humanized antibody.

Preferably the DNA sequence in steps (a) and (b) encode suitable constant domains from the human antibody chain. Suitable isotypes include, without limitation, IgG1 and IgG4, for example.

Alternatively, a suitable humanized antibody of the invention can be prepared by making a single replicable “mega” vector that includes an appropriate promoter operably linked to a DNA sequence which encodes at least a variable domain of an Ig heavy or light chain, the variable domain comprising each of the individually humanized framework regions (FR1-4) and murine CDRs 1-3 from a subject antibody.

In one embodiment, the mega vector will further include a suitable promoter operably linked to a DNA sequence which encodes at least the variable domain of a complementary Ig light or heavy chain respectively, that variable domain comprising corresponding and individually humanized framework regions (FR1-4) and murine CDRs 1-3 from the cH36 antibody or other suitable CDRs. Use of the mega vector will often be appropriate where humanized antibody expression from a single vector is needed.

It will be apparent that the foregoing method for making the humanized cH36 antibody can be readily adapted to make other humanized antibodies and antigen binding fragments according to the invention.

Particular examples of antibodies well suited for the humanization methods of this invention have been disclosed in the U.S. Ser. No. 09/990,586; and No. 60/343,306 applications as well as the Examples that follow.

By the words “assembling” or “assembled” is meant use of standard recombinant techniques to introduce subject DNA sequences encoding the humanized frameworks or framework regions into the vectors. Such assembly can be performed by one or combination of approaches including, but not limited to, introducing iterative changes to a single framework or framework region sequence, cutting and pasting fragments together (via use of restriction endonucleases and ligase), or by synthetic DNA synthesis techniques. See generally Harlow and Lane supra and Ausubel et al. supra.

The foregoing methods for making humanized antibodies can be practiced with nearly any acceptable mutagenesis technique. In particular, relevant method steps can employ site-directed mutagenesis and/or standard PCR methods to replace desired rodent amino acids in the framework with appropriate human amino acids. It can also be accomplished by DNA synthesis of modified fragments or entire coding regions, or by the in vitro recombination using standard recombinant DNA or genetic engineering techniques or any combination of these. Typically, the sequence of the modified (humanized) framework or framework region corresponds to the selected human framework or framework region sequence from the database.

Suitable nucleic acids of the invention encode at least one of the heavy or light chains of the humanized antibodies or fragments thereof disclosed herein. Typically, the nucleic acid is a recombinant DNA vector that includes the isolated nucleic acid. The DNA vector will typically further include an expression control polynucleotide sequence operably linked to the humanized immunoglobulin coding sequences, including naturally associated or heterologous promoter regions. Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high-level expression of the nucleotide sequences, and, as desired, the collection and purification of the light chains, heavy chains, light/heavy chain dimers or intact antibodies, binding fragments or other immunoglobulin forms may follow.

The nucleic acid sequences of the present invention capable of ultimately expressing the desired humanized antibodies can be formed from a variety of different polynucleotides (genomic or cDNA, RNA, synthetic oligonucleotides, etc.) and components (e.g., V, J, D, and C regions), as well as by a variety of different techniques. Joining appropriate genomic and synthetic sequences is presently the most common method of production, but cDNA sequences may also be utilized. See e.g., S. L. Morrison, supra; Oi et al., supra; Teng et al., supra; Kozbor et al., supra; Olsson et al., supra; European Patent Publication No. 0239400 and Riechmann, L. et al., Nature, 332: 323-327 (1988); and references cited therein.

Use of the invention is compatible with a wide range of suitable hosts e.g., mammalian, plant or microbial host cells. In one embodiment, suitable DNA expression vectors include one or more selection markers, e.g., tetracycline, ampicillin, geneticin, hygromycin, puromycin, or neomycin (or the like), to permit detection of those cells transformed with the desired DNA sequences (see, e.g., U.S. Pat. No. 4,704,362, which is incorporated herein by reference). E. coli is one prokaryotic host useful particularly for cloning the polynucleotides of the present invention. Other microbial hosts suitable for use include but are not limited to bacilli, such as Bacillus subtilus, and other Enterobacteriacea, such as Salmonella, Serratia, various Pseudomonas species and other microbes such as actinomycetes (e.g., Streptomyces species), yeast (e.g., Saccharomyces species) or fungi (e.g., Aspergillus species). In these prokaryotic hosts, one can also make expression vectors, which will typically contain expression control sequences compatible with the host cell (e.g., promoters and an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation. Other microbes, such as yeast, may also be used for expression. Saccharomyces is a preferred host, with suitable vectors having expression control sequences, such as promoters, including 3-phosphoglycerate kinase or other glycolytic enzymes, and an origin of replication, termination sequences and the like as desired.

In addition to forgoing microorganism-based systems, eukaryotic hosts may also be used to express and produce the polypeptides of the present invention (see, Winnacker, “From Genes to Clones”, VCH Publishers, N.Y., N.Y. (1987), which is incorporated herein by reference). In many embodiments, eukaryotic hosts will be generally preferred, typically mammalian cell lines without limitation, including CHO cell lines, various COS cell lines, NSO cells, BK cells, HeLa cells, preferably myeloma cell lines, etc., or transformed B-cells of hybridomas. Expression vectors for these cells can include expression control sequences, such as an origin of replication, a promoter, and enhancer (Queen et al., Immunol. Rev. 89: 46-68 (1986)), and necessary processing information sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, cytomegalovirus and the like. In other embodiments, eukaryotic hosts will be generally preferred where the eukaryotic host is a plant or plant cells without limitation, including e.g., Arabidopsis, Nicotinia, etc. and plant cell culture may also be used to express and produce the antibodies of the present invention. In other embodiments, eukaryotic hosts will be generally preferred where the eukaryotic host is an insect cell, avian species or a transgenic animal.

Preferred DNA vectors for practicing the invention include the following operatively linked sequences: an antibiotic resistance marker e.g., ampicillin resistance, F1 origin, and heavy chain (HC) or light chain (LC) variable domain. That variable domain can be inserted into an appropriate HC expression vector that includes operatively linked in sequence: the HC variable domain, human IgG1 or IgG4 constant domain, first polyA site, SV40 promoter, antibiotic resistance marker such as neomycin resistance, second polyA site, cytomegelovirus (CMV) promoter/enhancer, and suitable leader sequence.

Additionally preferred DNA vectors include the LC variable domain operatively linked to a rodent kappa intron (e.g., mouse) which intron is operatively linked to a suitable human kappa constant domain; and antibiotic resistance marker such a neomycin resistance.

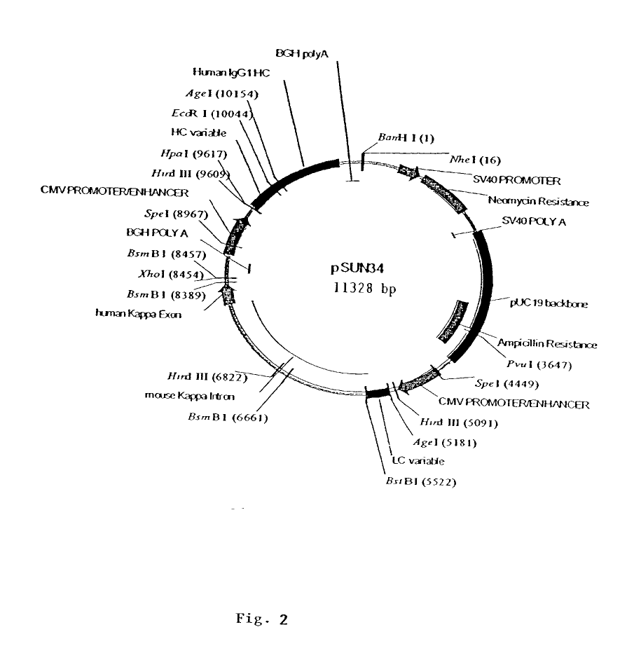

As discussed, it will often be highly useful to express humanized antibodies of the present invention from a single nucleic acid. A preferred DNA vector is sometime referred to herein as a “mega” vector and includes operatively linked in sequence the following components: SV40 promoter, antibiotic resistance marker such as neomycin, first polyA site, first CMV promoter/enhancer, LC variable domain, rodent kappa intron (e.g., mouse), human kappa exon, second polyA site, second CMV promoter/enhancer, HC variable domain, and human IgG1 or IgG4 heavy chain constant domain. A specific example of such a mega vector is the humanized anti-TF IgG1 antibody expression vector described below in Examples 1-3. See also FIG. 2

A. Preparation of Humanized Anti-Tissue Factor Binding Antibody

Preparation and use of a humanized anti-tissue factor binding antibody has already been described in the pending U.S. application Ser. No. 09/990,586 and No. 60/343,306. See also Examples 1-3 below.

Briefly, preferred antibodies bind human tissue factor to form a binding complex. The tissue factor may be naturally occurring or recombinant human (rhTF). Preferably, factor X or factor IX binding to the complex is inhibited. In a preferred invention embodiment, the humanized antibody has an apparent affinity constant (KA, M-1) for the hTF of less than about 1 nM, preferably less than about 0.5 nM, more preferably between from about 0.01 nM to about 0.4 nM. See Examples 1-3, below for more information about determining affinity constants for the humanized antibodies. By “specific binding” is meant that the humanized antibodies form a detectable binding complex with the TF (or rhTF) and no other antigen as determined by standard immunological techniques such as RIA, Western blot or ELISA.

More preferred humanized anti-TF binding antibodies made in accord with this invention exhibit an apparent affinity constant (KA, M-1) for native human TF of at least about 1×10⁸ M⁻¹ as determined by surface plasmon analysis (particularly, BIACore analysis in accordance with the procedures of Example 3 which follows), more preferably at least about 5×10⁸ M⁻¹ as determined by surface plasmon analysis, still more preferably an apparent affinity constant (K_{A}, M⁻¹) for native human TF of at least about 3×10⁹M⁻¹ as determined by surface plasmon resonance analysis. Such substantial binding affinity of antibodies of the invention contrast sharply from much lower binding affinities of previously reported antibodies.

In this regard, a quite low effective concentration of the humanized tissue factor binding antibody can be employed, e.g. a relatively low concentration of antibody can be employed to inhibit TF function as desired (e.g. at least about 95, 98 or 99 percent inhibition) in an in vitro assay such as described in Example 3 which follows.

The nucleic acid (SEQ ID NOS. 1 and 3) and amino acid (SEQ ID NOS. 2 and 4) sequences of a particular tissue factor binding antibody that has been humanized by the present methods i.e. H36.D2.B7. See FIGS. 1A and 1B of the drawings. SEQ ID NOS. 1 and 2 are the nucleic acid and amino acid respectively of the light chain variable domain, and SEQ ID NOS. 3 and 4 are the nucleic acid and amino acid respectively of the heavy chain variable domain, with hypervariable regions (CDRs or Complementarity Determining Regions) underlined in all of those sequences.

Additional tissue factor binding humanized antibodies of the invention will have substantial amino acid sequence identity to either one or both of the light chain or heavy sequences shown in FIGS. 1A and 1B. More particularly, such antibodies include those that have at least about 70 percent homology (amino acid sequence identity) to SEQ ID NOS. 2 and/or 4, more preferably about 80 percent or more homology to SEQ ID NOS. 2 and/or 4, still more preferably about 85, 90 or 95 percent or more homology to SEQ ID NOS. 2 and/or 4.

More particular tissue factor binding humanized antibodies of the invention will have high amino acid sequence identity to hypervariable regions (shown with double underlining in FIGS. 1A and 1B) of SEQ ID NOS. 2 and 4). Specific antibodies will have one, two or three hypervariable regions of a light chain variable domain that has high sequence identity (at least 90% or 95% amino acid sequence identity) to or be the same as one, two or three of the corresponding hypervariable regions of the light chain variable domain of H36.D2.B7 (those hypervariable regions shown with underlining in FIG. 1A and are the following:

1) LASQTID (SEQ ID NO. 5);

2) AATNLAD (SEQ ID NO. 6); and

3) QQVYSSPFT (SEQ ID NO. 7).

Additionally specific antibodies that have been humanized by the methods described herein and bind tissue factor will have one, two or three hypervariable regions of a heavy chain variable domain that have high sequence identity (at least 90% or 95% amino acid sequence identity) to or be the same as one, two or three of the corresponding hypervariable regions of the heavy chain variable domain of H36.D2.B7 (those hypervariable regions shown with underlining in FIG. 1B and are the following:

1) TDYNVY (SEQ ID NO. 8);

2) YIDPYNGITIYDQNFKG (SEQ ID NO. 9); and

3) DVTTALDF (SEQ ID NO. 10).

Certain nucleic acids of the invention preferably are of a length sufficient (preferably at least about 100, 200 or 250 base pairs) to bind to the sequence of SEQ ID NO. 1 and/or SEQ ID NO. 3 under the following moderately stringent conditions (referred to herein as “normal stringency” conditions): use of a hybridization buffer comprising 20% formamide in 0.9M saline/0.12M sodium citrate (6×SSC) buffer at a temperature of 37° C. and remaining bound when subject to washing once with that 2×SSC buffer at 37° C.

More specifically, certain nucleic acids of the invention (preferably at least about 100, 200 or 250 base pairs) will bind to the sequence of SEQ ID NO. 1 and/or SEQ ID NO. 3 under the following highly stringent conditions (referred to herein as “high stringency” conditions): use of a hybridization buffer comprising 20% formamide in 0.9M saline/0.12M sodium citrate (6×SSC) buffer at a temperature of 42° C. and remaining bound when subject to washing twice with that 1×SSC buffer at 42° C.

Nucleic acids of the invention preferably comprise at least 20 base pairs, more preferably at least about 50 base pairs, and still more preferably a nucleic acid of the invention comprises at least about 100, 200, 250 or 300 base pairs.

Generally preferred nucleic acids of the invention will express an antibody of the invention that exhibits the preferred binding affinities and other properties as disclosed herein.

Other nucleic acids of the invention also will have substantial sequence identity to either one or both of the light chain or heavy sequences shown in FIGS. 1A and 1B. More particularly, preferred nucleic acids will comprise a sequence that has at least about 70 percent homology (nucleotide sequence identity) to SEQ ID NOS. 1 and/or 3, more preferably about 80 percent or more homology to SEQ ID NOS. 1 and/or 3, still more preferably about 85, 90 or 95 percent or more homology to SEQ ID NOS. 1 and/or 3.

Additionally specific nucleic acid sequences of the invention will have high sequence identity to hypervariable regions (shown with underlining in FIGS. 1A and 1B) of SEQ ID NOS. 1 and 3). Such nucleic acids include those that code for an antibody light chain variable domain and have one, two or three sequences that code for hypervariable regions and have high sequence identity (at least 90% or 95% nucleotide sequence identity) to or be the same as one, two or three of the sequences coding for corresponding hypervariable regions of H36.D2.B7 (those hypervariable regions shown with underlining in FIG. 1A and are the following:

1) CTGGCAAGTCAGACCATTGAT (SEQ ID NO: 11);

2) GCTGCCACCAACTTGGCAGAT (SEQ ID NO: 12); and

3) CAACAAGTTTACAGTTCTCCATTCACGT (SEQ ID NO: 13).

More specific nucleic acids also code for an antibody heavy chain variable domain and have one, two or three sequences that code for hypervariable regions and have high sequence identity (at least 90% or 95% sequence identity) to or be the same as one, two or three of the sequences coding for corresponding hypervariable regions of H36.D2.B7 (those hypervariable regions shown with underlining in FIG. 1B and are the following:

1) ACTGACTACAACGTGTAC (SEQ ID NO. 14);

2) TATATTGATCCTTACAATGGTATTACTATCTACGACCAGAACTTCAAGGGC (SEQ ID NO. 15); and

3) GATGTGACTACGGCCCTTGACTTC (SEQ ID NO. 16).

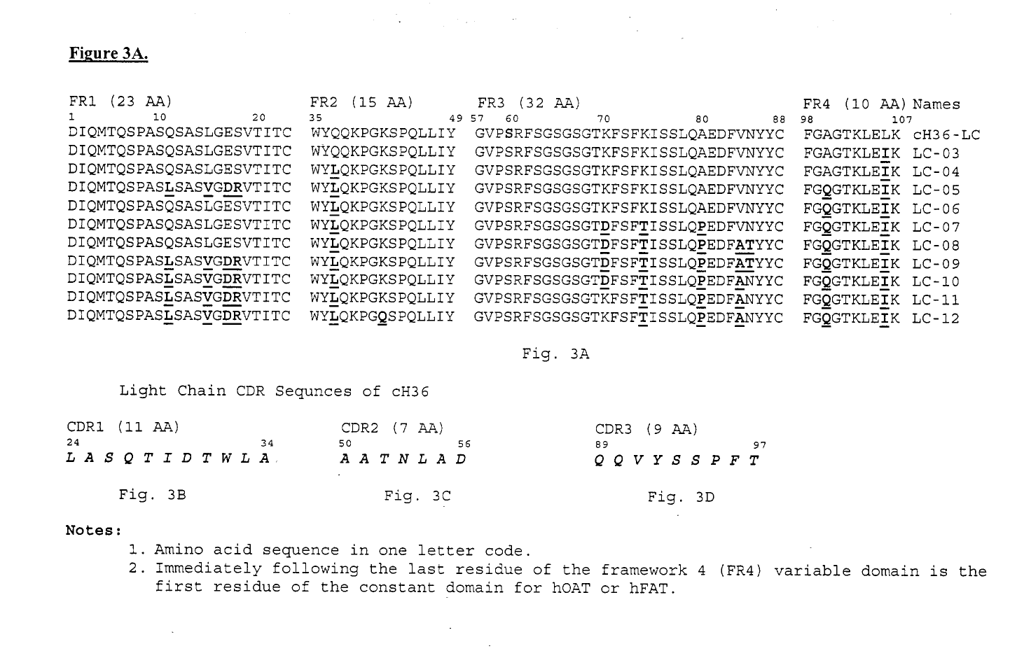

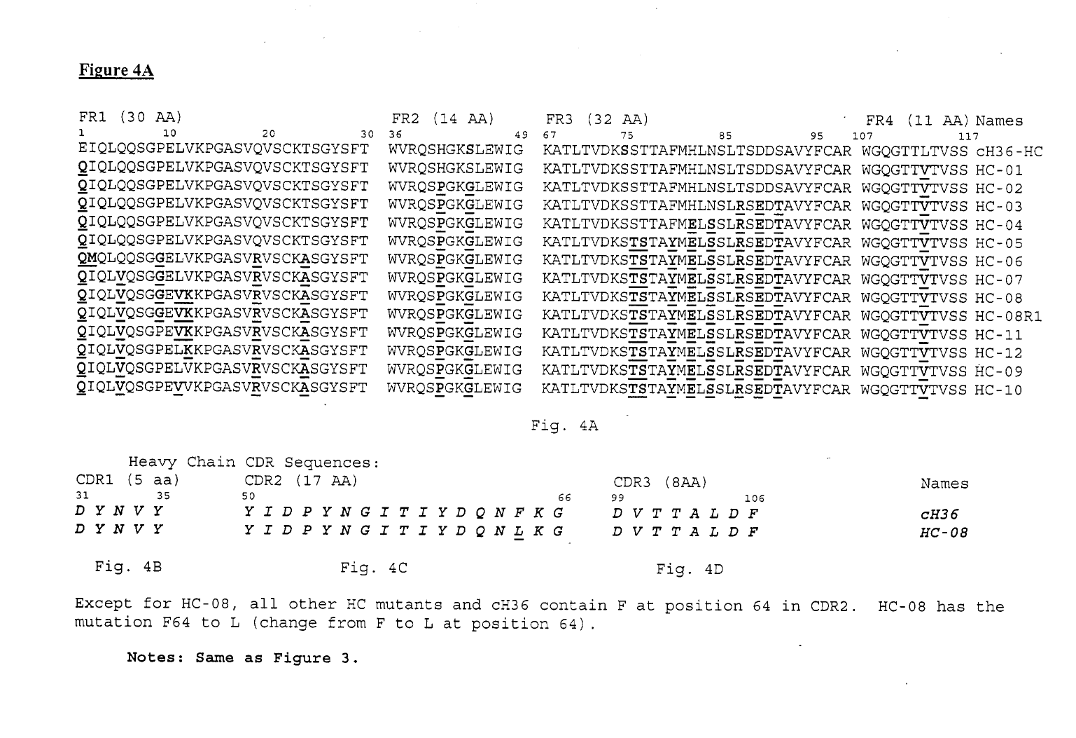

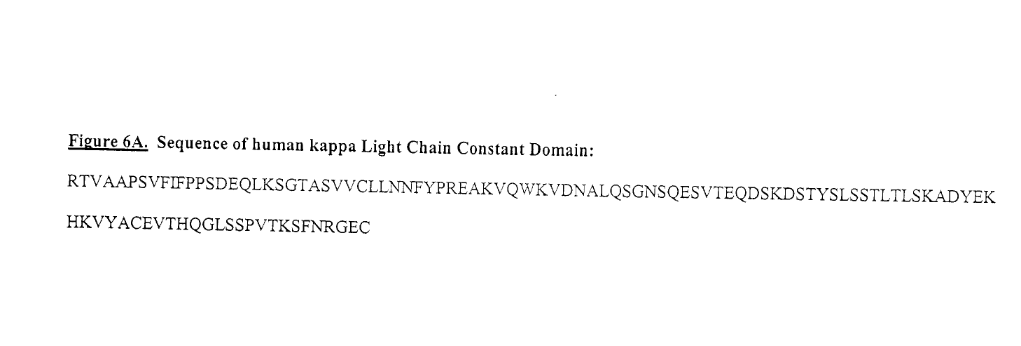

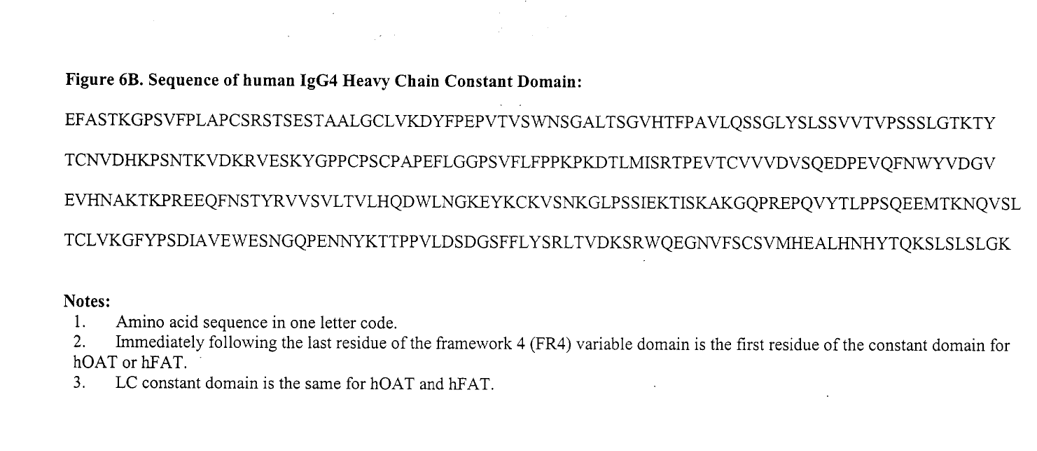

More specific humanized antibodies of the invention that bind TF are those in which each of framework regions (FRs) 1, 2, 3 and 4 has at least about 90% amino acid sequence identity, preferably at least about 95% or greater identity to the light chain FR sequences shown in FIG. 3A (SEQ ID NO. [symbol] ), preferably, the sequence shown as “LC-09” in FIG. 3A. Additionally specific humanized antibodies include a light chain constant domain having at least about 90% amino acid sequence identity, preferably at least about 95% sequence identity or greater to the sequence shown in FIG. 5A (SEQ ID NO. [symbol] ) or FIG. 6A (SEQ ID NO. [symbol] ).

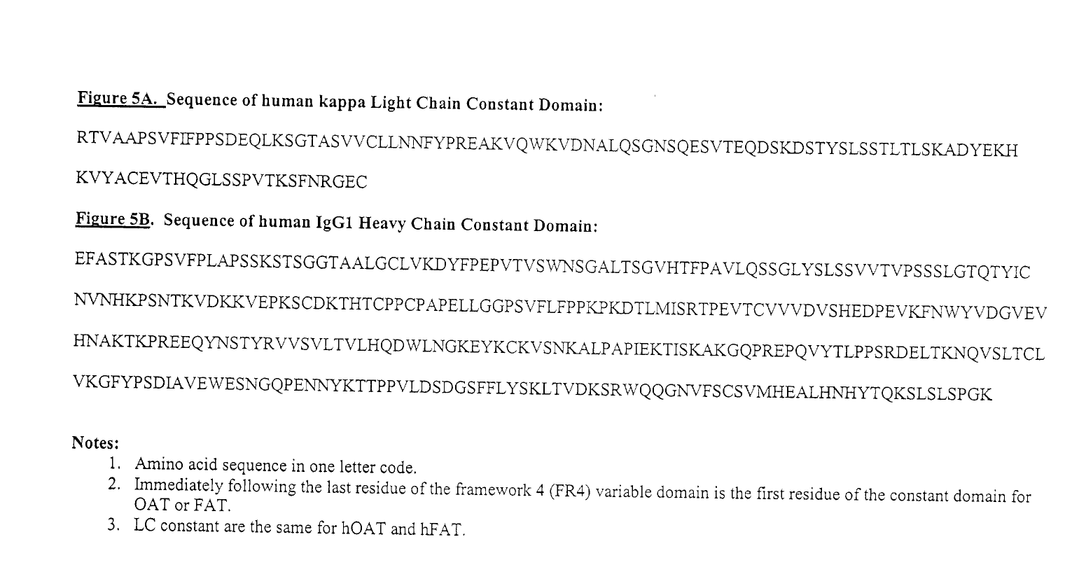

Further specific humanized antibodies are those in which each of framework regions (FRs) 1, 2, 3 and 4 has at least about 90% amino acid sequence identity, preferably about 95% identity or greater to the heavy chain sequences shown in FIG. 4A (SEQ ID NO. [symbol] ) preferably, the sequence shown as “HC-08” in FIG. 4A. Additional humanized antibodies have a heavy chain constant domain with at least about 90% amino acid sequence identity, preferably at least about 95% identity or greater, to sequence shown in FIG. 5B (SEQ ID NO. [symbol] ) or FIG. 6B (SEQ ID NO. [symbol] ).

In certain embodiments, the humanized antibody will have an IgG1 (hOAT) or IgG4 (hFAT) isotype as disclosed in the pending U.S. application Ser. No. 09/990,586 and No. 60/343,306.

Also provided by the present invention are functional fragments of the humanized antibodies disclosed herein. Examples of such fragments include, but are not limited to, those that bind TF with an affinity constant (Kd) of less than about 1 nM, preferably less than about 0.5 nM, more preferably between from about 0.01 nM to about 0.4 nM. Specifically preferred are antigen binding Fab, Fab′, and F(ab)₂ fragments.

As discussed, the invention features humanized antibodies that include at least one murine complementarity determining region (CDR), e.g., CDR1, CDR2, CDR3. In one invention embodiment, the antibodies bind specifically to human tissue factor (TF) to form a complex. Typically, the factor X or factor IX binding to TF or TF:FVIIa and activation by TF:FVIIa thereto is inhibited. As mentioned above, preferred CDRs (light and heavy chain) are from a rodent source, typically the mouse.

In one embodiment of the humanized antibodies of the invention, the antibodies further include at least one human framework region (FR) subset. Preferably, all the FRs (light and heavy chains) are human.

In a more particular embodiment, the first CDR (CDR1) of the heavy chain hypervariable region that binds human TF is at least 90% identical to the CDR1 amino acid sequence shown in FIG. 4B (SEQ ID NO. [symbol] ), preferably at least about 95% identical or greater to that sequence. Typically, the second CDR (CDR2) of the heavy chain hypervariable region is at least 90% identical to the CDR2 amino acid sequence shown in FIG. 4C (SEQ ID NO. [symbol] ), preferably at least about 95% identical or greater. Preferably also, the third CDR (CDR3) of the heavy chain hypervariable region is at least 90% identical to the CDR3 sequence shown in FIG. 4D (SEQ ID NO. [symbol] ), more preferably about 95% identical or greater to that sequence.

In another invention embodiment, the first CDR (CDR1) of the light chain hypervariable region that binds human TF is at least 90% identical to the CDR1 amino acid sequence shown in FIG. 3B (SEQ ID NO. [symbol] ), preferably at least about 95% identical or greater. Typically, the second CDR (CDR2) of the light chain hypervariable region is at least 90% identical to the CDR2 amino acid sequence shown in FIG. 3C (SEQ ID NO. [symbol] ), preferably about 95% identical or greater. Preferably, the third CDR (CDR3) of the light chain hypervariable region is at least 90% identical to the CDR3 amino acid sequence shown in FIG. 3D (SEQ ID NO. [symbol] ), more preferably about 95% identical or greater to that sequence.

Additional humanized antibodies of the invention include a first framework region (FR1) of the heavy chain hypervariable region that binds human TF which FR1 is at least 90% identical to the amino acid sequence shown in FIG. 4A (SEQ ID NO. [symbol] ) as “FR1 HC-08”, preferably about 95% identical or greater to that sequence. In one embodiment, the FR1 comprises at least one of the following amino acid changes: E1 to Q; Q5 to V; P9 to G; L11 to V; V12 to K; Q19 to R; and T24 to A. Preferably, the FR1 includes two, three, four, five, or six of those changes with all of those amino acid changes being preferred for many applications.

Further humanized antibodies of the invention that suitably bind human TF include a second framework region (FR2) of the heavy chain hypervariable region which FR2 is at least 90% identical to the sequence shown in FIG. 4A (SEQ ID NO. [symbol] ) as “FR2 HC-08”, preferably about 95% identical or greater to that sequence. In one embodiment, the FR2 at least one of the following amino acid changes: H41 to P; and S44 to G. A preferred FR2 includes both of those amino acid changes.

The invention also features humanized antibodies that bind human TF in which a third framework region (FR3) of the heavy chain hypervariable region is at least 90% identical to the sequence shown in FIG. 4A (SEQ ID NO. [symbol] ) as “FR3 HC-08”, preferably about 95% identical or greater to that sequence. In one embodiment, the FR3 includes at least one of the following amino acid changes: S76 to T; T77 to S; F80 to Y; H82 to E; N84 to S; T87 to R; D89 to E; and S91 to T. A preferred FR3 includes two, three, four, five or six of those amino acid changes with all seven of those amino acid changes being generally preferred.

Also featured are humanized antibodies that suitably bind human TF in which the fourth framework region (FR4) of the heavy chain hypervariable region is at least 90% identical to the amino acid sequence shown in FIG. 4A (SEQ ID No. [symbol] ) as “FR4 HC-08”, preferably at least about 95% identical or greater to that sequence. Preferably, the FR4 includes the following amino acid change: L113 to V.

Additional humanized antibodies in accord with the invention that bind human TF feature a first framework region (FR1) of the light chain hypervariable region which is at least about 90% identical to the amino acid sequence shown in FIG. 3A (SEQ ID NO. [symbol] ) as “FR1 LC-09”, preferably at least about 95% identical or greater to that sequence. In one embodiment, the FR1 comprises at least one of the following amino acid changes: Q11 to L; L15 to V; E17 to D; and S18 to R. A preferred FR1 includes two or three of such amino acid changes with all four amino acid changes being generally preferred.

The present invention also features humanized antibodies that bind human TF in which a second framework region (FR2) of the light chain hypervariable region is at least about 90% identical to the amino acid sequence shown in FIG. 3A (SEQ ID NO. [symbol] ) as “FR2 LC-09”, preferably at least about 95% identical or greater to that sequence. A preferred FR2 has the following amino acid change: Q37 to L.

Also encompassed by the invention are humanized antibodies that bind human TF in which a third framework region (FR3) of the light chain hypervariable region is at least about 90% identical to the amino acid sequence shown in FIG. 3A (SEQ ID NO. [symbol] ) as “FR3 LC-09”, preferably at least about 95% identical or greater to that sequence. In one embodiment, the FR3 has at least one of the following amino acid changes: K70 to D, K74 to T, A80 to P, V84 to A, and N85 to T. Preferably, the FR3 has two, three, or four of such amino acid changes with all five of the changes being generally preferred.

Additional humanized antibodies of the invention that bind TF include a fourth framework region (FR4) of the light chain hypervariable region which FR4 is at least about 90% identical to the sequence shown in FIG. 3A (SEQ ID NO. [symbol] ) as “FR4 LC-09”, preferably at least about 95% identical or greater to that sequence. In one embodiment, the FR4 includes at least one and preferably all of the following amino acid changes: A100 to Q; and L106 to I.

The invention also features a human TF binding fragment of the foregoing humanized antibodies. Examples of such fragments include Fab, Fab′, and F(ab)₂.

See the U.S. Ser. No. 09/990,586 and No. 60/343,306 patent applications for additionally preferred humanized anti-TF antibodies made in accord with this invention. As disclosed therein, the following three nucleic acid vectors pSUN36 (humanized anti-TF antibody Ig GI-HC expression vector), pSUN37 (humanized anti-TF antibody Ig G4-HC expression vector), and pSUN38 (humanized anti-TF antibody LC expression vector) have been deposited pursuant to the Budapest Treaty with the American Type Culture Collection (ATCC) at 10801 University Boulevard, Manassas Va. 20110-2209. The vectors were assigned the following Accession Numbers: PTA-3727 (pSUN36); PTA-3728 (pSUN37); and PTA-3729 (pSUN38).

Suitable expression and purification strategies for making and using the humanized anti-TF antibodies of this invention have been disclosed in the U.S. Ser. No. 09/990,586 and No. 60/343,306 patent applications.

B. Preparation of Humanized Anti-LTA Binding Antibody

The original anti-LTA chimeric antibody is the subject of the following patent applications: “Opsonic and Protective Monoclonal and Chimeric Antibodies specific of Lipotechoic Acid of Gram Positive Bacteria” Pending U.S. Ser. No. 09/097,055 (Published as WO 98/57994)

Preparation and use of a humanized anti-LTA (lipotechoic acid) binding antibody is described below in Examples 4-5. Preferred antibodies generally bind LTA to form a specific binding complex. Particular chimeric anti-LTA antibodies bind antigen with an apparent affinity constant (KA, M-1) of less than about 1 μM, preferably less than about 100 nM, more preferably between from about 20 nM to about 2 nM. See Example 5, below for further information about characterizing anti-LTA antibodies.

Lipotechoic acid is a cell component found in some Gram-positive bacteria including Staphylococcus species, some Streptococcus species and Entercoccus. It is incorporated into the cell wall as part of a mixed macromolecular polymer that is heterodisperse in molecular weight, and as such the LTA component can be found in cell walls and fragments thereof that can have an extremely broad molecular mass range.

More preferred humanized anti-LTA binding antibodies an association constant (K_{A}, M⁻¹) for LTA of at least about 5×10⁶ M⁻¹ as determined by surface plasmon analysis (particularly, BIACore analysis in accordance with the procedures of Example 3 which follows), more preferably at least about 1×10⁷ M⁻¹ as determined by surface plasmon analysis, still more preferably a Kₐ for LTA of at least about 1×10⁸ M⁻¹ as determined by surface plasmon resonance analysis.

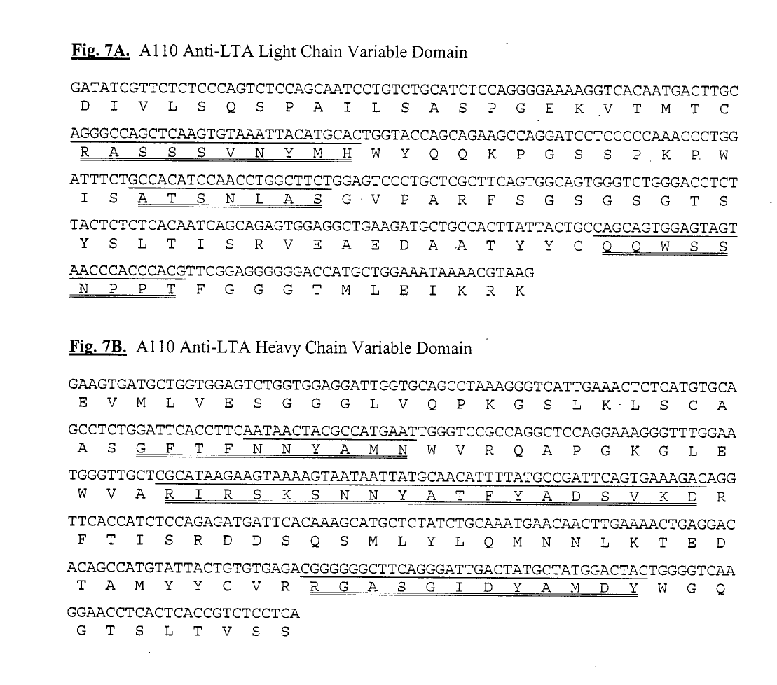

In one embodiment, the first CDR (CDR1) of the light chain hypervariable region that binds the LTA antigen is at least about 90% identical to the CDR1 amino acid sequence shown in FIG. 7A (underlined) preferably at least about 95% or greater identity. Typically, the second CDR (CDR2) of the same light chain hypervariable region is at least about 90% identical to the CDR2 amino acid sequence shown in FIG. 7A (underlined), preferably at least about 95% or greater identity. Preferably also, the third CDR (CDR3) of the light chain hypervariable region is at least about 90% identical to the CDR3 amino acid sequence shown in FIG. 7A (underlined), more preferably at least about 95% or greater sequence identity.

With respect to the heavy chain hypervariable region, additionally preferred anti-LTA antibodies will exhibit at least about 90% identity to the CDR1 amino acid sequence shown in FIG. 7B (underlined), preferably at least about 95% or greater identity. Typically, the second CDR (CDR2) of the same light chain hypervariable region is at least about 90% identical to the CDR2 amino acid sequence shown in FIG. 7B (underlined), preferably at least about 95% or greater identity. Preferably also, the third CDR (CDR3) of the light chain hypervariable region is at least about 90% identical to the CDR3 amino acid sequence shown in FIG. 7B (underlined), more preferably at least about 95% or greater sequence identity.

Additionally specific anti-LTA antibodies of the invention include those in which each of light chain framework region subsets (FRs) 1, 2, 3 and 4 has at least about 90% amino acid sequence identity, preferably at least about 95%, 98% up to 100% identity to each of the light chain FR sequences shown in Table 6 of Example 5. In one embodiment, the framework includes at least one and preferably all of the following amino acid changes: D1 to Q; S5 to T; L11 to M; E17 to D; M21 to I; S41 to Q; R76 to A; V77 to M; and M102 to K. An especially preferred anti-LTA antibody has at least one of and preferably all of each of the L chain FR subsets shown for the A110-LC in Table 6 of Example 5 i.e., LC-FR1 (SEQ ID NO. ______); LC-FR2 (SEQ ID NO. ______); LC-FR3 (SEQ ID NO. ______) and LC-FR4 (SEQ ID NO. ______).

Further specific humanized antibodies that specifically bind the LTA antigen include those in which each of framework regions (FRs) 1, 2, 3 and 4 has at least about 90% amino acid sequence identity, preferably about 95%, 98% up to about 100% identity to the heavy chain sequences shown in Table 7 of Example 5. In one embodiment, the framework includes at least one and preferably all of the following amino acid changes: M3 to Q; K15 to G; Q78 to K; S79 to N; M80 to S; N87 to S; M95 to V, V99 to A and L119 to V. An especially preferred anti-LTA antibody has at least one of and preferably all of each of the H chain FR subsets shown for the A110-HC in Table 7 of Example 5. That is, HC-FR1 (SEQ ID NO. ______); HC-FR2 (SEQ ID NO. ______); HC-FR3 (SEQ ID NO. ______); and HC-FR4 (SEQ ID NO. ______).

In certain embodiments, the humanized anti-LTA antibody will have an IgG1 heavy chain constant domain (similar to hOAT) or an IgG4 heavy chain constant domain (similar to hFAT) isotype. See the pending the U.S. Ser. No. 09/990,586 and No. 60/343,306 patent applications.

Additionally preferred anti-LTA antibodies will have each of the light and heavy chain FRs shown in Tables 6 and 7 of Example 5.

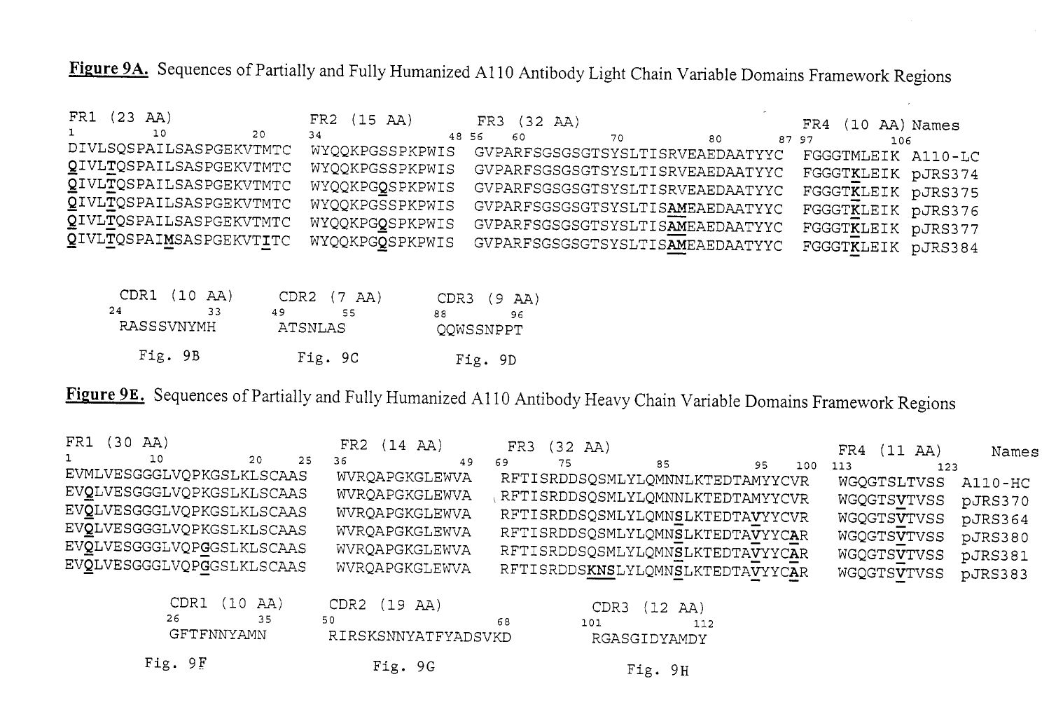

Further preferred humanized anti-LTR antibodies will have an amino acid light chain variable domain with at least 95% sequence identity, preferably at least about 98% or greater identity to the amino acid sequence shown in FIG. 9A (SEQ ID NO. ______). More preferably, such an antibody will have a light chain variable domain that is the same as the sequence shown in FIG. 9A (SEQ ID NO. ______). Additionally preferred antibodies will have an amino acid heavy chain variable domain with at least 95% sequence identity, preferably at least about 98% or greater identity to the amino acid sequence shown in FIG. 9E (SEQ ID NO. ______). More preferably, such an antibody will have a heavy chain variable domain that is the same as the sequence shown in FIG. 9E (SEQ ID NO. ______).

In a particular example of the invention, there is provided a humanized anti-LTA antibody that specifically binds LTA and includes at least one rodent CDR, usually from a mouse. Preferably, the LTA antigen binds specifically to the antibody to form a complex. Also preferably, such a humanized anti-LTA antibody includes, on the heavy chain, at least one of and preferably all of the following components:

a) a first CDR (CDR1) which is at least 95% identical to CDR1 amino acid sequence shown in FIG. 9F (SEQ ID NO. [symbol] ),

b) a second CDR (CDR2) which is at least 95% identical to the CDR2 amino acid sequence shown in FIG. 9G (SEQ ID NO. [symbol] ),

c) a third CDR (CDR3) which is at least 95% identical to the CDR3 amino acid sequence shown in FIG. 9H (SEQ ID NO. [symbol] ),

d) a first framework subset (FR1) which is at least 95% identical to the amino acid sequence shown in Table 6A (SEQ ID NO. [symbol] ) as “LC-FR1”,

e) a second framework subset (FR2) which is at least 95% identical to the amino acid sequence shown in Table 6A (SEQ ID NO. [symbol] ) as “LC-FR2”,

f) a third framework subset (FR3) which is at least 95% identical to the amino acid sequence shown in Table 6B (SEQ ID NO. [symbol] ) as “LC-FR3”, and

g) a fourth framework subset (FR4) which is at least 95% identical to the amino acid sequence shown in Table 6B (SEQ ID No. [symbol] ) as “LC-FR4”.

In a particular embodiment, the humanized anti-LTA antibody also includes, on the light chain, at least one of and preferably all of the following components:

h) a first CDR (CDR1) which is at least 95% identical to CDR1 amino acid sequence shown in FIG. 9B (SEQ ID NO. [symbol] ),

i) a second CDR (CDR2) which is at least 95% identical to the CDR2 amino acid sequence shown in FIG. 9C (SEQ ID NO. [symbol] ),

j) a third CDR (CDR3) which is at least 95% identical to the CDR3 amino acid sequence shown in FIG. 9D (SEQ ID NO. [symbol] ),

k) a first framework subset (FR1) which is at least 95% identical to the amino acid sequence shown in Table 7A (SEQ ID NO. [symbol] ) as “HC-FR1”,

l) a second framework subset (FR2) which is at least 95% identical to the amino acid sequence shown in Table 7A (SEQ ID NO. [symbol] ) as “HC-FR2”,

m) a third framework subset (FR3) which is at least 95% identical to the amino acid sequence shown in Table 7B (SEQ ID NO. [symbol] ) as “HC-FR3”, and

n) a fourth framework subset (FR4) which is at least 95% identical to the amino acid sequence shown in Table 7B (SEQ ID NO. [symbol] ) as “HC-FR4. Preferably, the humanized antibody further includes the light chain constant sequence of FIG. 6A (HFAT (IgG4) SEQ ID NO. [symbol] ). Also preferably, the antibody includes the heavy chain constant region of FIG. 6B (IgG4 SEQ ID NO. [symbol] ).

The invention further encompasses nucleic acid molecules that encode one or more of the amino acid anti-LTA light chain variable domain and anti-LTA heavy chain variable domain shown in FIG. 9A (SEQ ID NO. ______) and 9E (SEQ ID NO. ______), respectively. More specific nucleic acids express at least part of an anti-LTA binding antibody that exhibits the preferred binding affinities and other properties disclosed herein. The molecular weight of such nucleic acids will generally be less than about 1000 basepairs (bp), preferably between from about 200 bp to about 750 bp, as determined by conventional gel electrophoresis methods.

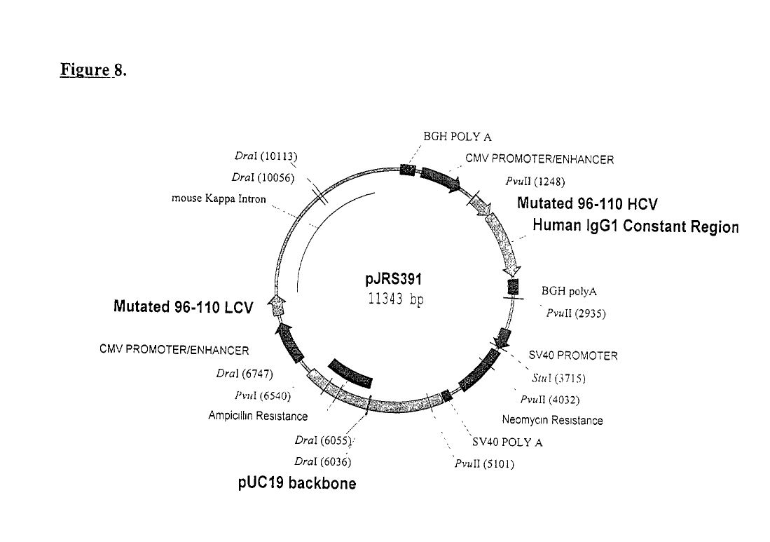

A specifically preferred nucleic acid according to the invention is the plasmid represented by FIG. 8 (PJRS 391).

Also provided by the present invention are functional fragments of the humanized anti-LTA antibodies disclosed herein. Examples of such fragments include, but are not limited to, those that bind LTA with an apparent affinity constant (KA, M-1) of less than about 100 nM, preferably less than about 25 nM, more preferably between from about 1 nM to about 5 nM. Specifically preferred are antigen binding Fab, Fab′, and F(ab)₂ fragments, single chain Fv and full length antibodies.

In general, nucleic acids of the invention are isolated, usually constitutes at least about 0.5%, preferably at least about 2%, and more preferably at least about 5% by weight of total nucleic acid present in a given fraction. A partially pure nucleic acid constitutes at least about 10%, preferably at least about 30%, and more preferably at least about 60% by weight of total nucleic acid present in a given fraction. A pure nucleic acid constitutes at least about 80%, preferably at least about 90%, and more preferably at least about 95% by weight of total nucleic acid present in a given fraction.

The following non-limiting examples are illustrative of the invention. In the following examples and elsewhere the method of the invention is applied to the humanization of the murine anti-tissue factor antibody H36 and the anti-lipotechoic acid antibody A110. H36 is also referred to as H36.D2 and as H36.D2.B7, but H36 is the antibody produced by the mother clone, and H36.D2 is obtained from the primary clone, whereas H36.D2.B7 is obtained from the secondary clone. No differences have been observed between the antibody produced by those three clones with respect to the antibody's ability to inhibit TF or other physical properties. In general usage, H36 is often used to indicate anti-TF antibody produced by any of these clones or related cell lines producing the antibody. The mouse-human chimeric version of H36 is referred to cH36 (and also as Sunol-cH36). The anti-lipotechoic acid antibody A110 is also referred to as 96-110, c96-110 and as BSYX-A110 and is a mouse-human chimeric antibody.

See the U.S. Ser. No. 09/990,586 and No. 60/343,306 patent applications for further disclosure relating to making and using the H36 antibody. See also U.S. Pat. No. 5,986,065.

All documents mentioned herein are fully incorporated by reference in their entirety.

### EXAMPLE 1

### Humanization of Anti-Tissue Factor Antibody

The description of how to make and use a particular murine antibody called H36.D2 (sometimes also called H36 as discussed above) is described in U.S. Pat. No. 5,986,065. The present example shows how to make and use a humanized version of that antibody. A humanized H36 antibody has a variety of uses including helping to minimize potential for human anti-mouse antibody (HAMA) immunological responses. These and other undesired responses pose problems for use of the H36 antibody in human therapeutic applications.

A. Preparation of Chimeric Anti-Tissue Factor Antibody (cH36)

The H36 antibody described previously is an IgG2a murine antibody. H36 was first converted to a mouse-human chimeric antibody for clinical development. To do this, the heavy and light chain genes for H36 were cloned (see U.S. Pat. No. 5,986,065). The heavy chain variable domain was fused to a human IgG4 constant (Fc) domain and the light chain variable domain was fused to a human kappa light chain constant domain. The resulting IgG41c chimeric antibody was designated cH36 (and is also referred to as Sunol-cH36). For multiple uses of H36 or cH36 in patients with chronic diseases, a fully humanized cH36 is preferred so that it will decease or eliminate any human anti-chimeric antibody (HACA) immunological response. The humanization of cH36 is described below.

B. Humanization Strategy for cH36 Antibody

Humanization of the chimeric anti-tissue factor antibody cH36 was achieved by using a “FR best-fit” method of the invention. This method takes full advantage of the fact that a great number of human IgGs with known amino acid sequences or sequences of human IgG fragments are available in the public database. The sequences of the individual framework regions of the mouse heavy and light variable domains in cH36 are compared with the sequences respective heavy or light chain variable domains or human frameworks (or fragments thereof) in the Kabat database (see http://immuno.bme.nwu.edu). The following criteria were used to select the desired human IgG framework region subsets for humanization: (1) The number of mismatched amino acids was kept as low as possible. (2) Amino acids inside the “vernier” zone (amino acids in this zone may adjust CDR structure and fine-tune the fit to antigen, see Foote, J. and Winter, G., J. of Mol. Bio. 224(2): 487-499 [1992]) were left unchanged. (3) Conservative amino acid substitutions were favored when evaluating similar candidates. The matching program used for this comparison can be found in Kabat database. See Johnson G, Wu T. “Kabat database and its application: Future directions.” Nucleic Acids Res. (2001) 29:205-206. The program finds and aligns regions of homologies between the mouse sequences and human sequences in the Kabat's database. By using this unique FR best-fit method, it is anticipated that the humanized LC or HC variable domains of the target IgG may have all the four FRs derived from as few as one human IgG molecule or to as many as four different human IgG molecules.

B(i). Selection of Human IgG Kappa Light Chain Variable Domain Framework Regions

The amino acid sequence in each of the framework regions of cH36 LC was compared with the amino acid sequence in the FRs in human IgG kappa light chain variable domain in Kabat Database. The best-fit FR was selected based on the three criteria described above.

The amino acid sequence of human IgG kappa light chain variable domain with a Kabat Database ID No. 005191 was selected for humanization of cH36 LC FR1. The amino acid sequence of human IgG kappa light chain variable domain with a Kabat Database ID No. 019308 was selected for humanization of cH36 LC FR2. The following mutations were made in cH36 LC FR1 to match the amino acid sequence of a human IgG kappa light chain variable domain with a Kabat Database ID No.005191: Q11 L, L15 V, E17 D, S18 R. One mutation Q37 L was made cH36 LC FR2 to match the amino acid sequence of a human IgG kappa light chain variable domain with a Kabat Database ID No. 019308 (see Table 1A for sequence information).

The amino acid sequence of a human IgG kappa light chain variable domain with a Kabat Database ID No. 038233 was selected for humanization of cH36 LC FR3. The amino acid sequence of a human IgG kappa light chain variable domain with a Kabat Database ID No. 004733 was selected for humanization of cH36 LC FR4. The following mutations were made in cH36 LC FR3 to match the amino acid sequence of a human IgG kappa light chain variable region with a Kabat Database ID No. 038233: K70 D, K74 T, A80 P, V84 A, N85 T. Two mutations A100 Q and L106 I were made cH36 LC FR4 to match the amino acid sequence of a human IgG kappa light chain variable domain with a Kabat Database ID No. 004733 (see Table 1B for sequence information).

B(ii). Selection of Human IgG Heavy Chain Variable Domain Framework Regions

The amino acid sequence in each of the framework regions of cH36 HC was compared with the amino acid sequence in the FRs in human IgG heavy chain variable domain in Kabat Database. The best-fit FR was selected based on the three criteria described above.

The amino acid sequence of a human IgG heavy chain variable domain with a Kabat Database ID No. 000042 was selected for humanization of cH36 HC FR1. The amino acid sequence of a human IgG heavy chain variable domain with a Kabat Database ID No. 023960 was selected for humanization of cH36 HC FR2. The following mutations were made in cH36 HC FR1 to match the amino acid sequence of a human IgG heavy chain variable domain with a Kabat Database ID No. 000042: E1 Q, Q5 V, P9 G, L11 V, V12 K, Q19 R, T24 A. Two mutations H41 P and S44 G were made cH36 HC FR2 to match the amino acid sequence of a human IgG heavy chain variable domain with a Kabat Database ID No. 023960 (see Table 2A for sequence information).

The amino acid sequence of a human IgG heavy chain variable domain with a Kabat Database ID No. 037010 was selected for humanization of cH36 HC FR3. The amino acid sequence of a human IgG heavy chain variable domain with a Kabat Database ID No. 000049 was selected for humanization of cH36 HC FR4. The following mutations were made in cH36 HC FR3 to match the amino acid sequence of a human IgG heavy chain variable domain with a Kabat Database ID No. 037010: S76 T, T77 S, F80 Y, H82 E, N84 S, T87 R, D89 E, S91 T. One mutations L113 V was made cH36 HC FR2 to match the amino acid sequence of a human IgG heavy chain variable domain with a Kabat Database ID No. 000049 (see Table 2B for sequence information).

TABLE 1

Comparison of cH36 and Human Light Chain (LC) FR Se-
quences
Table 1A

| Names | LC-FR1 (23 aa) | LC-FR2 (15 aa) |
|---|---|---|
| | 1 10 20 | 35 49 |
| cH36-LC | DIQMTQSPASQSASLGESVTITC | WYQQKPGKSPQLLIY |
| Human-LC | L V DR | L |
| | 005191 | 019308 |

Table 1B

| Names | LC-FR3 (32 aa) | LC-FR4 (10 aa) |
|---|---|---|
| | 57 60 70 80 88 | 98 107 |
| cH36 -LC | GVPSRFSGSGSGTKFSFKISSLQAEDFVNYYC | FGAGTKLELK |
| Human-LC | D T P AT | Q I |
| | 038233 | 004733 |

TABLE 2

Comparison of cH36 and Human Heavy Chain (HC) FR Se-
quences
Table 2A

| Names | HC-FR1 (30 aa) | HC-FR2 (14 aa) |
|---|---|---|
| | 1 10 20 30 | 36 49 |
| cH36 -HC | EIQLQQSGPELVKPGASVQVSCKTSGYSFT | WVRQSHGKSLEWIG |
| Human-HC | Q V G VK R A | P G |
| | 000042 | 023960 |

Table 2B

| Names | HC-FR3 (32 aa) | HC-FR4 (11 aa) |
|---|---|---|
| | 67 75 85 95 | 107 117 |
| cH36 -HC | KATLTVDKSSTTAFMHLNSLTSDDSAVYFCAR | WGQGTTLTVSS |
| Human-HC | TS Y E S R E T | V |
| | 037010 | 000049 |

Once the decisions on the desired human framework regions were made, the following three techniques were used to achieve the desired amino acid substitutions in both the light and heavy chains: (1) Regular PCR was used for cloning, to introduce cloning or diagnostic restriction endonuclease sites, and to change amino acid residues located at the ends of the variable domains. (2) PCR-based mutagenesis was used to change multiple amino acid residues at a time, especially when these residues were in the middle of the variable domains. (3) Site-directed mutagenesis was used to introduce one or two amino acid substitutions at a time. Site-directed mutagenesis was done following the protocol described in Stratagene's “QuickChange Site-Directed Mutagenesis Kit” (Catalog #200518).

After each step, the partially humanized clones were sequenced and some of these variable domains were later cloned into expression vectors. The plasmid tKMC180 was used to express LC mutants, and pJRS 355 or pLAM 356 vector was used to express HC mutants as IgG1 or IgG4, respectively. Some of these clones were then combined and expressed transiently in COS cells to determine the expression levels by ELISA.

The final fully humanized forms of the anti-TF heavy and light variable domains were cloned into what is sometimes referred to herein as a “mega vector” and transfected into CHO and NSO cells for IgG expression. Stable cell lines were then used to produce amounts of humanized anti-TF sufficient for analysis. The resulting humanized versions are 100% human in origin (when the CDR sequences are not considered). The humanized IgG4 kappa version is designated HFAT (humanized IgG Four Anti-Tissue Factor antibody) and the IgG1 kappa version is designated hOAT (humanized IgG One Anti-Tissue Factor antibody). These fully humanized versions of cH36 are intended for treating chronic indications, such as thrombosis, cancer and inflammatory diseases.

C. Generation of Humanized Anti-TF Antibody Heavy Chain

1. PCR amplification and cloning into pGem T-easy of anti-TF mAb cH36 heavy chain (HC) variable domain were performed using plasmid pJAIgG4TF.A8 (an expression vector for chimeric H36) as template and primers TFHC1s2 and TFHC1as2. Primer TFHC1s2 introduced a BsiW1 site upstream of the initiation codon and also an amino acid change E1 to Q in framework (FR) 1. Primer TFHC1as introduced an amino acid change L113 to V in FR4. This step resulted in the construct HC01.

2. PCR-based mutagenesis using the previous construct (HC01) and the following four primers generated construct HC02. Upstream PCR used primers TFHC1s2 and TFHC7 as. Downstream PCR used primers TFHC7s and TFHC1as2. PCR using upstream and downstream PCR products as templates and with primers TFHC1s2 and TFHC1as2 yielded HC02. The use of primers TFHC7s and TFHC7 as introduced two amino acid changes in FR2: H41 to P and S44 to G.

3. PCR-based mutagenesis using HC02 as template and the following four primers generated construct HC03. Upstream PCR used primers TFHC1s2 and TFHC5as2. Downstream PCR used primers TFHC5s and TFHC1as2. PCR using upstream and downstream PCR products as templates and with primers TFHC1s2 and TFHC1as2 yielded HC03. The use of primers TFHC5s and TFHC5 as2 introduced three amino acid changes in FR3: T87 to R, D89 to E, and S91 to T. A Bgl II site was also introduced at position. 87.

4. PCR amplification was performed using primers TFHC2s and TFHC3 as and HC03 in pGem as template. TFHC2s sits upstream of the cloning site in pGem. TFHC3 as sits in framework 3 and introduces two amino acid changes in FR3: H82 to E and N84 to S. The resulting PCR band was cloned into pGem and then the proper size insert was digested with BsiW1 and Bgl II. Cloning of this fragment into HC03 yields HC04.

5. PCR-based mutagenesis using HC04 as template and the following primers resulted in HC05. Upstream PCR used primers TFHC1s2 and TFHC6 as. Downstream PCR used primers TFHC6s and TFHC1as2. Mutagenic PCR using upstream and downstream PCR products as templates and with primers TFHC1s2 and TFHC1as2 yielded HC05. This step introduced the following amino acid changes in FR3: S76 to T, T77 to S, and F80 to Y.

6. PCR-based mutagenesis using HC05 as template and the following four primers generated HC06. Upstream PCR used primers TFHC2s and TFHC2 as2. Downstream PCR used primers TFHC3s2 and TFHC1as2. Amplification using TFHC2 as2 introduced an amino acid change in FR1: P9 to G. Primer TFHC3s2 changes Q19 to R and T24 to A. PCR using upstream and downstream PCR products as template and with primers TFHC1s2 and TFHC1as2 yielded HC06.

7. A point mutation from 1 to M in position 2 of FR1 was spontaneously introduced during construction of HC06. PCR amplification using HC06 as template and TFHC1s3 and TFHC1as2 as primers, corrected this erroneous substitution and also introduced an amino acid. change in FR1: Q5 to V. The resulting construct was HC07.

8. Construct HC08 was made by PCR-based mutagenesis using HC07 as template and the following primers. TFHC2s and TFHC2 as3 were used for the upstream product. The downstream product was previously amplified using TFHC1s3 and TFHC1as2 (see step 7). The use of primer TFHC2 as3 introduced two amino acid changes in FR1: L11 to V and V12 to K. A spontaneous point mutation resulted in a phenylalanine to leucine (F→L) change at position 64 in CDR2. Further screening and sequencing yielded construct HC08R1, which has the correct sequence of F at position 64 in CDR2.

9. Two constructs, HC11 and HC12, were generated by site-directed mutagenesis from HC07. Two complementary primers TFHC8sP and TFHC8asP were used along with HC07 as template to produce HC11 which contains three amino acid changes in FR1: G9 to P, L11 to V, and V12 to K. Then, HC11 was methylated and column purified for the next round of site directed mutagenesis. PCR using HC11 as a template and the complementary primers TFHC9sL and TFHC0asL generated HC12 which has a mutation from V11 to L in FR1.

10. Construct HC09 was derived from HC12 by performing PCR using HC12 as a template and the complementary primers TFHC10sK and TFHC10asK. HC09 contains an amino acid change: K12 to V in FR1.

11. Construct HC10 was made from HC09. PCR using HC09 as a template and the complementary primers LV-1 and LV-2 resulted in the generation of HC10, which contains a mutation from L11 to V in FR1.

After each mutation step, the partially humanized or fully humanized clones were sequenced and some of these variable domains were later cloned into expression vectors. pJRS 355 or pLAM 356 vector was used to express HC mutants fused to Fe of human IgG1 or IgG4. FIGS. 3A-D summarize steps 1-11 and shows incremental amino acid changes introduced into FR1-4. Except HC08, all other heavy chain mutants and cH36 contain F at position 64 in CDR2. HC08 has a mutation from F to L at position 64. FIGS. 4B-D show the heavy chain CDR sequences.

Primers Used for Heavy Chain Humanization

TFHC1s2
5′ TTTCGTACGTCTTGTCCCAGATCCAGCTGCAGCAGTC 3′
TFHC1as2
5′ AGCGAATTCTGAGGAGACTGTGACAGTGGTGCCTTGGCCCCAG 3′
TFHC7s
5′ GTGAGGCAGAGCCCTGGAAAGGGCCTTGAGTGGATTGG 3′
TFHC7as
5′ CCAATCCACTCAAGGCCCTTTCCAGGGCTCTGCCTCAC 3′
TFHC5s
5′GCATCTCAACAGCCTGAGATCTGAAGACACTGCAGTTTATTTCTGTG 3′
TFHC5as2
5′ CTGCAGTGTCTTCAGATCTCAGGCTGTTGAGATGCATGAAGGC 3′
TFHC3as
5′ GTCTTCAGATCTCAGGCTGCTGAGCTCCATGAAGGCTGTGGTG 3′
TFHC2s
5′ TACGACTCACTATAGGGCGAATTGG 3′
TFHC6s
5′ CTGTTGACAAGTCTACCAGCACAGCCTACATGGAGCTCAGCAG 3′
TFHC6as
5′ CTGCTGAGCTCCATGTAGGCTGTGCTGGTAGACTTGTCAACAG 3′
TFHC2as2
5′ GCACTGAAGCCCCAGGCTTCACCAGCTCACCTCCAGACTGCTGCAGC 3′
TFHC3s2
5′CTGGGGCTTCAGTGCGGGTATCCTGCAAGGCTTCTGGTTACTCATTCAC 3′
TFHC1s3
5′ TCGTACGTCTTGTCCCAGATCCAGCTGGTGCAGTCTGGAGGTGAGC 3′
TFHC2as3
5′ GCACTGAAGCCCCAGGCTTCTTCACCTCACCTCCAGACTGCACC 3′
TFHC9sL
5′ GCAGTCTGGACCTGAGCTGAAGAAGCCTGGGG 3′
TFHC9asL
5′ CCCCAGGCTTCTTCAGCTCAGGTCCAGACTGC 3′
TFHC8sP
5′ GCTGGTGCAGTCTGGACCTGAGGTGAAGAAGCC 3′
TFHC8asP
5′ GGCTTCTTCACCTCAGGTCCAGACTGCACCAGC3′
TFHC10sK
5′ GCAGTCTGGACCTGAGCTGGTGAAGCCTGGGGCTTC 3′
TFHC10asK
5′ GAAGCCCCAGGCTTCACCAGCTCAGGTCCAGACTGC 3′
LV-1
5′ CAGTCTGGACCTGAGGTGGTGAAGCCTGGG 3′
LV-2
5′ CCCAGGCTTCACCACCTCAGGTCCAGACTG 3′

D. Generation of Humanized Anti-TF Antibody Light Chain

1. PCR amplification was performed using plasmid pJAIgG4TF.A8 (an expression vector for chimeric H36) as template and primers TFLC1s2.1 and TFLC1as2. This step introduced a cloning site, AgeI, upstream of the coding region. It also introduced the L1061 mutation in FR4. This step yielded the construct LC03.

2. Site-directed mutagenesis was performed using complementary primers TFLC5s and TFLC5 as and LC03 as template. This step introduced the mutation Q37L in FR2 and added a PstI site for diagnostic purposes. This new construct is named LC04.

3. PCR amplification was performed using LC04 as template and primers TFHC2s and TFLC2as1. This step generated Fragment A that will be used in step 6. This step introduced Q11L and L15V mutations in FR1.

4. PCR amplification was performed using LC04 as template and primers TFLC1s2.1 and TFLC1asR. This introduced the KpnI site at the end of LC variable domain. Cloning of this PCR fragment into pGEM yields pGEM04K that will be used in step 6.

5. PCR amplification was performed using LC04 as template and primers TFLC2s and TFLC4 as. This step generated Fragment C that will be used in step 6. Three mutations E17D, S18R in FR1 and A100Q in FR4 were introduced in this step.

6. PCR-based mutagenesis using Fragment A and Fragment C as templates and primers TFHC2s and TFLC4 as yielded Fragment D. Cloning of Fragment D into pGEM04K yielded the construct LC05.

7. PCR amplification was performed using pGEM04K as template and primers TFLC1s2.1 and TFLC4 as. This step generated Fragment H, which is then cloned into pGEM04K. This introduced the A100Q mutation in FR4 and the construct is named LC06.

8. PCR amplification was performed using LC06 as template and primers TFLC1s2.1 and TFLC3 as. This step generated Fragment I that will be used in step 10. This introduced the K70D and the K74T mutations in FR3.

9. PCR amplification was performed using LC06 as template and primers TFLC3s2 and TFLC4 as. This step generated Fragment F that will be used in step 10. This introduced the A80P mutation in FR3.

10. PCR using Fragment I and Fragment F as templates and primers TFLC1s2.1 and TFLC4 as yielded Fragment J. Cloning of Fragment J into pGEM yielded the construct LC07.

11. Site-directed mutagenesis was conduced using complementary primers TFLC08sds and TFLC08sdsa and LC07 as template. This step introduced the mutations V84A and N85T in FR3. This construct is named LC08.

12. The Agel to EcoO109I fragment from LC05 containing the mutations Q11L, L15V, E17D, S18R and Q37L is cloned into LC08. This yielded the construct LC09.

13. Site-directed mutagenesis was conduced using LC09 as template and the complementary primers LC105 and LC103. This step introduced the T85N mutation in FR3 and yielded the construct LC10.

14. Site-directed mutagenesis was conducted using LC10 as template and the complementary primers LC115 and LC113. This step introduced the D70K mutation in FR3. This yielded the construct LC11.

15. Site-directed mutagenesis was conducted using LC11 as template and the complementary primers LC125a and LC123a. This step introduced the K42Q mutation in FR2. This yielded the construct LC12.

After each mutation step, the partially humanized or fully humanized LC clones were sequenced and some of these variable domains were later cloned into expression vector tKMC180.

Oligonucleotide Primers Used for Light Chain Humanization

TFLC1as2:
5′ TTCGAAAAGTGTACTTACGTTTGATCTCCAGCTTGGTCCCAG 3′
TFLC1s2.1:
5′ ACCGGTGATATCCAGATGACCCAGTCTCC 3′
TFLC5s:
5′ GGTTAGCATGGTATCTGCAGAAACCAGGG 3′
TFLC5as:
5′ CCCTGGTTTCTGCAGATACCATGCTAACC 3′
TFHC2s:
5′ TACGACTCACTATAGGGCGAATTGG 3′
TFLC2as1:
5′ CCACAGATGCAGACAGGGAGGCAGGAGACTG 3′
TFLC1asR:
5′ TTCGAAAAGTGTACTTACGTTTGATCTCCAGCTTGGTACCAGCACCGAACG 3′
TFLC2s:
5′ CCTGTCTGCATCTGTGGGAGATAGGGTCACCATCACATGC 3′
TFLC4as:
5′ GATCTCCAGCTTGGTACCCTGACCGAACGTGAATGG 3′
TFLC3as:
5′ GTAGGCTGCTGATCGTGAAAGAAAAGTCTGTGCCAGATCC 3′
TFLC3s2:
5′ CACGATCAGCAGCCTACAGCCTGAAGATTTTGTAAATTATTACTGTC 3′
TFLC08sds:
5′ GCAGCCTACAGCCTGAAGATTTTGCAACTTATTACTGTCAACAAG 3′
TFLC08sdsa:
5′ CTTGTTGACAGTAATAAGTTGCAAAATCTTCAGGCTGTAGGCTGC 3′
LC105:
5′ CAGCAGCCTACAGCCTGAAGATTTTGCAAATTATTACTGTCAAC 3′
LC103:
5′ GTTGACAGTAATAATTTGCAAAATCTTCAGGCTGTAGGCTGCTG 3′
LC115:
5′ CAGTGGATCTGGCACAAAGTTTTCTTTCACGATCAGCAGC 3′
LC113:
5′ GCTGCTGATCGTGAAAGAAAACTTTGTGCCAGATCCACTG 3′
LC125a:
5′ CTGCAGAAACCAGGGCAATCTCCTCAGCTCCTG 3′
LC123a:
5′ CAGGAGCTGAGGAGATTGCCCTGGTTTCTGCAG 3′

FIG. 5A shows the sequence of the human kappa light chain constant domain (SEQ ID NO. ______). FIG. 5B shows the human IgG1 heavy chain constant domain (SEQ ID NO. ______). FIG. 6A shows the hFAT (IgG4) constant domain sequence (SEQ ID NO. ______). FIG. 6B provides the human IgG4 heavy chain constant domain (SEQ ID NO. ______). See also the U.S. Ser. No. 09/990,586 and No. 60/343,306 for additional disclosure relating to the foregoing immunoglobulin constant domain sequences.

### EXAMPLE 2

### Expression and Purification of Humanized anti-TF Antibodies

The partially humanized or fully humanized LC and HC clones were cloned into expression vectors. The plasmid tKMC18 was used to express LC mutants fused to human kappa chain, and pJRS 355 or pLAM 356 vector was used to express HC mutants fused to Fc of human IgG1 or IgG4. Some combinations of the HC and LC clones were then co-transfected into COS cells. The transiently expressed IgGs in COS cells were assayed for the whole IgG production and binding to TF by ELISA. For disclosure relating to these particular vectors see the pending U.S. application Ser. No. 09/990,586 and No. 60/343,306.

The final fully humanized forms of the anti-TF heavy and light variable domains (combination of HC08 and LC09) were cloned into what is referred to as a Mega expression vector (pSUN34, see FIG. 2) and transfected into CHO and NSO cells for IgG expression. Stably transfected cell lines producing the IgG4K or IgGIK humanized anti-TF antibody were cloned. The selected stable cell lines were then used to produce amounts of humanized anti-TF sufficient for analysis. The resulting humanized versions are approximately 100% human in origin (when the CDR sequences are not considered). The humanized IgG4 kappa version (produced by pSUN35) is designated HFAT (humanized IgG Four Anti-Tissue Factor antibody) and the IgG1 kappa version (produced by pSUN34) is designated hOAT (humanized IgG One Anti-Tissue Factor antibody). These fully humanized versions of cH36 are intended for treating chronic indications, such as cancer and inflammatory diseases.

One of the NSO cell lines (OAT-NSO-P10A7) that expresses hOAT (combination of HC08 and LC09) was thawed and extended in 10 mL of IMDM medium supplemented with 10% FBS in a 15 mL tube and centrifuged. The cell pellet was resuspended in 10 mL of fresh media and passed to a T25 flask and incubated at 37° C. in 5% CO₂. In order to prepare a sufficient number of cells to inoculate a hollow fiber bioreactor, the cells were expanded to obtain a total of 6×10⁸ cells. A bioreactor was set up as per manufacturer's instruction manual. The harvested cells were pelleted and resuspended in 60 mL of IMDM containing 35% FBS and injected into the extracapillary space of the bioreactor. Concentrations of glucose and lactate were monitored daily and the harvest material was centrifuged and pooled. The harvested material was tested for anti-TF antibody concentrations by ELISA assay. The pooled sample containing anti-TF antibody (hOAT) were then purified and analyzed as described below.

A. rProtein A Sepharose Fast Flow Chromatography

Recombinant humanized anti-TF monoclonal antibody consists of two light and two heavy chains. Heavy chain is a fusion of mouse variable domain (unaltered or humanized as described above) and human IgG1 or IgG4 Fc domain, while light chain contains mouse variable domain (unaltered or humanized as described above) and human K domain. It is well established that human IgG Fc region has high affinity for Protein A or recombinant Protein A (rProtein A).

Harvest pools containing humanized anti-TF antibody (hOAT) were adjusted to pH 8.0±0.1 by adding 0.08 ml of 1 M Tris-HCl, pH 8.0 per ml of sample. Then the sample is filtered through low protein-binding 0.22 micron filters (e.g., Nalgene sterile disposable tissue culture filter units with polyethersulfone membrane from Nalge Nunc International, Cat. No. 167-0020). Following sample application, rProtein A column (from Pharmacia) is washed with 5 bed volumes of 20 mM Tris-HCl, pH 8.0 to remove unbound materials such as media proteins. Since the harvest medium contains high content of bovine serum, a stepwise pH gradient wash was used to remove bovine IgG from the column. The stepwise pH gradient was achieved by increasing the relative percentage of Buffer B (100 mM acetic acid) in Buffer A (100 mM sodium acetate). A typical pH stepwise wash employed 20%, 40%, and 60% Buffer B. Elute the column with 100% Buffer B and collect fractions based on A₂₈₀. The pooled fractions were adjusted to pH 8.5 with addition of 1 M Tris base.

B. Q Sepharose Fast Flow Chromatography

Anion ion exchange chromatography is very effective in separating proteins according to their charges. The eluted and pH-adjusted sample from rProtein A column was diluted with two volumes of water, and the pH is checked and adjusted to 8.5. The sample was then loaded to a 5 ml (1.6×2.5 cm) Q Sepharose Fast Flow equilibrated with 20 mM Tris-HCl, pH 8.5 and the column washed with (1) 5 bed volumes of 20 mM Tris-HCl, pH 8.5; and (2) 4 bed volumes of 20 mM Tris-HCl, pH 8.5 containing 100 mM NaCl. The IgG protein was then eluted with bed volumes of 20 mM Tris-HCl, pH 8.5 containing 500 mM NaCl. The protein peaks were pooled and buffer-exchanged into PBS using ultrafiltration device.

Using the same transfection, cell culture, and purification methods, hFAT was also produced and purified.

### EXAMPLE 3

### Properties of Humanized Anti-TF Antibodies

A. Inhibition of TF Function by Humanized Anti-TF Antibody

One of the key properties of anti-TF antibodies is its ability to inhibit tissue factor-initiated blood coagulation. The purified hOAT and hFAT were measured for their ability to inhibit TF activity in a standard PT assay. PT assay is widely used to measure tissue factor-dependent blood clotting times. The principal of this assay is that tissue factor (TF) forms complex with factor VIIa in plasma. This complex then activates factor X to FXa; FXa then converts prothrombin to thrombin in the presence of factor Va and phospholipids. Thrombin eventually leads to formation of a blood clot. In standard PT assays, lipidated TF is added to plasma to initiate blood coagulation and the clotting is recorded by an Organon Teknika Coag-A-Mate Coagulation Analyzer or equivalent.

The anti-TF antibody, H36, inhibits human TF activity by a unique mechanism. It binds to TF (free or in complex with factor VIIa) in such a way that factor X and IX binding to TF:FVIIa complex is prohibited, thus FX and FIX activation by TF:FVIIa is blocked (see U.S. Pat. No. 5,986,065). In PT tests, the prolongation of clotting times anti-TF antibody added into human plasma is a clear indication that this TF-dependent coagulation is inhibited. The clotting time is related to the amount of TF activity. A TF standard curve is generated by measuring PT clotting times of serially diluted TF. From the data of TF standard curve, the inhibition of TF activity by anti-TF antibody is determined.

Reagents: Innovin (Cat No 68100-392) and Ci-Trol Coagulation Control, Level I (Cat No 68100-336) are obtained from VWR. Lipidated recombinant human TF was produced as described in Example 3 in U.S. Pat. No. 5,986,065.

Method: PT test is performed at 37 C using a Coagulation Analyzer. PT reaction is initiated by adding 0.2 ml of lipidated recombinant human tissue factor (e.g., Innovin) into 0.1 ml of human plasma (Ci-Trol Control Level I) containing 0.01 ml buffer (50 mM Tris-HCl, pH 7.5, 0.1% BSA) or anti-TF antibody.

1. Add purified water to a vial of Innovin according to manufacturer's instruction. Warm the reagent to 37° C. The reagent is stable for a few days if stored at 4-8° C.

2. Add 1 ml purified water to each vial of Ci-Trol. Mix to solubilize. If more one vials are used, combine them into one container (e.g., a 10 ml test tube). 1 ml Ci-Trol can run 5 assays (each assay uses 2×0.1 ml=0.2 ml). Ci-Trol can be stored on ice and last for a few hours.

3. From anti-TF antibody stock, make a series of anti-TF antibody solutions (200 nM to 1600 nM) with 50 mM Tris-HCl, pH 7.5, 0.1% BSA

4. Add 10 μl of 50 mM Tris-HCl, pH 7.5, 0.1% BSA or 10 μl of diluted anti-TF to each well of the twin-well cuvette that contains 0.1 ml of Ci-Trol. Use a pipette with 0.1 ml tip to mix each well. Make sure no air bubbles are in the well. Following mixing anti-TF (or buffer) with plasma (Ci-Trol), measure clotting times within 10 min by adding 0.2 ml of Innovin to the plasma.

5. For TF standard curve, first dilute Innovin (100% TF) to 20%, 10%, 5%and 2.5% with 50 mM Tris-HCl, pH 7.5, 0.1% BSA. Then PT assays were performed as in Step 4 but using diluted Innovin samples.

Table 3 is the summary of the effect of cH36, hOAT, and HFAT on PT clotting times. Compared to the data in Table 4, cH36, HFAT, and hOAT showed very potent inhibition of TF function. At a protein concentration of above 12.9 nM, all antibodies achieved about 95% inhibition. The results in Table 3 also indicate that humanization of anti-TF, cH36, by the method described above did not have any significant effect on cH36 inhibitory activity since both HFAT and hOAT showed very similar ability to inhibit TF-dependent blood coagulation as seen for cH36.

TABLE 3
Effect on Prothrombin Times by Chimeric (cH36) and
Humanized) Anti-TF Antibodies (hFAT and hOAT)^{#}

| Anti-TF Antibody | | | |
|---|---|---|---|
| Concentrations (nM) in | PT Time (in seconds) | | |
| PT Assays | cH36 | hOAT | hFAT |
| 0 | 12.2 | 12.2 | 12.2 |
| 6.45 | 14.9 | nd | nd |
| 9.7 | 17.8 | 16.5 | nd |
| 12.9 | 19.8 | 18.9 | 20.5 |
| 25.8 | 40 | 33.7 | 41.7 |
| 51.6 | 101.3 | 82.1 | 94.8 |

^{#}All assays used the same 100% TF activity (concentration) sample as in TABLE 4.

TABLE 4
Clotting Times and Relative Tissue Factor
Activities (Concentrations)

| Relative TF Activities (Concentrations) | PT Clotting Times (Seconds) |
|---|---|
| 100% (neat) | 11.90 |
| 20% | 13.225 |
| 10% | 14.675 |
| 5% | 16.700 |
| 2.5% | 20.000 |

B. Determination of Affinity Constants

The affinity of humanized anti-TF antibody for TF was determined by surface plasmon resonance (BIAcore from Pharmacia Biosensor) with recombinant human tissue factor covalently immobilized on a CM5 sensor chip. The affinity constants were the average data calculated from four anti-TF monoclonal antibody concentrations (0.125 nM, 0.25 nM, 0.5 nM, and 1 nM) by the BIAcore computer software. The results in Table 5 indicate that humanization of anti-TF, cH36, by the method described above did not have any significant effect on cH36 affinity for TF since both cH36 and hFAT have similar affinity for TF.

TABLE 5
Apparent Affinity and Dissociation Constants of Anti-TF Antibodies

| Anti-TF Antibody | Apparent K_{A} (M⁻¹) | Apparent K_{d} (M) |
|---|---|---|
| H36 | 1.56 × 10¹⁰ | 6.4 × 10⁻¹¹ |
| cH36 | 7.94 × 10⁹ | 1.26 × 10⁻¹⁰ |
| hFAT | 2.99 × 10⁹ | 3.35 × 10⁻¹⁰ |

### EXAMPLE 4

### Humanization of Anti-LTA Antibody

A. Chimeric anti-LTA Antibody

B. Humanization Strategy for Anti-LTA Antibody

Humanization of the chimeric anti-LTA (lipoteichoic acid) antibody, A110, was achieved by using an “FR best-fit” method. This method takes full advantage of the fact that a great number of human IgG variable domains with known amino acid sequences are available in the public database. The individual frameworks of the mouse heavy and light variable domains in A110 are compared with their corresponding human frameworks in the Kabat database (see http://immuno.bme.nwu.edu). The following criteria were used to select the desired human IgG frameworks for humanization. (1) The number of mismatched amino acids was kept as low as possible. (2) Amino acids inside the “vernier” zone (amino acids in this zone may adjust CDR structure and fine-tune the fit to antigen, see Foote, J. and Winter, G., J. Mol. Bio. 224; (2) 487-499 [1992]) were left unchanged. (3) Conservative amino acid substitutions were favored when evaluating similar candidates. The matching program used for this comparison can be found in Kabat's internet home page. The program finds and aligns regions of homologies between the mouse sequences and human sequences in the database. By using this unique best-fit method, it is anticipated that the humanized LC or HC variable domain of the target IgG may have all the four FRs derived from as few as one human IgG molecule to as many as four human IgG molecules.

B (i). Selection of Human IgG Kappa Light Chain Variable Domain Frameworks

The amino acid sequence in each of the frameworks of A110 LC was compared with the amino acid sequence in the corresponding FR in human IgG kappa light chain variable domain in Kabat Database. The best-fit FR was selected based on the three criteria described above.

The amino acid sequence of human IgG kappa light chain variable domain with a Kabat Database ID No. 036047 was selected for humanization of A110 LC FR1 and FR3. The amino acid sequence of human IgG kappa light chain variable domain with a Kabat Database ID No. 037658 was selected for humanization of A110 LC FR2 and No. 004763 was selected for humanization of FR4. The following mutations were made in A110 LC to match the amino acid sequence of a human IgG kappa light chain variable domain with selected FRs: D1 Q, S5 T, L11 M, M21 I, S42 Q, R76 A, V77 M, and, M102 K. (see Table 6 for sequence information).

B (ii). Selection of Human IgG Heavy Chain Variable Domain Frameworks

The amino acid sequence in each of the frameworks of A110 HC was compared with the amino acid sequence in the corresponding FR in human IgG heavy chain variable domain in Kabat Database. The best-fit FR was selected based on the three criteria described above.

The amino acid sequence of a human IgG heavy chain variable domain with a Kabat Database ID No. 000468 is selected for humanization of A110 HC FR1. The amino acid sequence of a human IgG heavy chain variable domain with a Kabat Database ID No. 000565 is selected for humanization of A110 HC FR2. The amino acid sequence of a human IgG heavy chain variable domain with a Kabat Database ID No. 000628 is selected for humanization of A110 HC FR3. The amino acid sequence of a human IgG heavy chain variable domain with a Kabat Database ID No. 031571 is selected for humanization of A110 HC FR4. The following mutations were made in A110 HC FR1 to match the amino acid sequence of a human IgG heavy chain variable domain with a Kabat Database ID No. 000468: M3 Q, and, K15 G. No changes were necessary for A110 HC FR2 in order to match the Kabat Database ID No. 000565 FR2 sequence. Six mutations were made A110 HC FR3 to match the amino acid sequence of a human IgG heavy chain variable domain with a Kabat Database ID No. 000628: Q78 K, S79 N, M80 S, N87 S, M95 V, and, V99 A. One mutation, L119 V was made Al 10 HC FR4 to match the amino acid sequence of a human IgG heavy chain variable domain with a Kabat Database ID No. 031571 (see Table 7 for sequence information).

TABLE 6

Comparison of A110 and Human Light Chain (LC) FR Se-
quences
Table 6A

| Names | LC-FR1 (23 aa) | LC-FR2 (15 aa) |
|---|---|---|
| | 1 10 20 | 34 48 |
| A110-LC | DIVLSQSPAILSASPGEKVTMTC | WYQQKPGSSPKPWIS |
| Human-LC | Q T M D I | Q |
| | 036047 | 037658 |

Table 6B

| Names | LC-FR3 (32 aa) | LC-FR4 (10 aa)- |
|---|---|---|
| | 56 60 70 80 87 | 97 106 |
| A110 -LC | GVPARFSGSGSGTSYSLTISRVEAEDAATYYC | FGGGTMLEIK |
| Human-LC | AM | K |
| | 036047 | 004763 |

TABLE 7

Comparison of A110 and Human Heavy Chain (HC) FR Se-
quences
Table 7A

| Names | HC-FR1 (25 aa) | HC-FR2 (14 aa) |
|---|---|---|
| | 1 10 20 | 36 49 |
| A110 -HC | EVMLVESGGGLVQPKGSLKLSCAAS | WVRQAPGKGLEWVA |
| Human-HC | Q G | unchanged |
| | 000468 | 000565 |

Table 7B

| Names | HC-FR3 (32 aa) | HC-FR4(11 aa) |
|---|---|---|
| | 69 75 85 95 100 | 113 123 |
| A110 -HC | RFTISRDDSQSMLYLQMNNLKTEDTAMYYCVR | WGQGTSLTVSS |
| Human-HC | KNS S V A | V |
| | 000628 | 031571 |

Once the decision on the desired human frameworks were made, the following three techniques were used to achieve the desired amino acid substitutions in both the light and heavy chains. (1) Regular PCR was used for cloning, to introduce cloning or diagnostic endonuclease sites, and to change amino acid residues located at the ends of the variable domains. (2) Mutagenic PCR was used to change multiple amino acid residues at a time, especially when these residues were in the middle of the variable domains. (3) Site directed mutagenesis was used to introduce one or two amino acid substitutions at a time. Site directed mutagenesis was done following the protocol described in Stratagene's “QuickChange Site-Directed Mutagenesis Kit” (Catalog #200518).

After each step, the partially humanized clones were sequenced and some of these variable domains were later cloned into expression vectors. The plasmid tKMC 180 was used to express LC mutants, and pJRS355 was used to express HC mutants as IgG1. Some of these clones were then combined and expressed transiently in COS cells.

The final fully humanized forms of the anti-LTA heavy and light variable domains were cloned into what is sometimes referred to herein as a “mega vector” and transfected into COS cells for IgG expression and LTA binding analysis.

C. Generation of Humanized Anti-LTA Antibody Heavy Chain

1. PCR amplification and cloning into pGEM T-easy (Promega) of anti-LTA mAb A110 heavy chain (HC) variable domain were performed using plasmid pJRS334 (an expression vector for A110) as template and primers HChuF1 and HChuR2. Primer HChuF1 introduced a BsiW1 site upstream of the first codon of the variable domain and also an amino acid change M3 to Q in framework (FR) 1. Primer HChuR2 introduced an amino acid change L119 to V in FR4 and a C-terminal EcoRI restriction site for cloning purposes. This step resulted in the construct pJRS362. This fragment was then sub-cloned into the expression vector pJRS355 as a BsiWI to EcoRI restriction fragment resulting in pJRS370.

2. PCR amplification of anti-LTA mAb A110 heavy chain (HC) variable domain fragments was performed using plasmid pJRS334 (an expression vector for A110) as template and primers HChuF1 and HChuR1 for the N-terminal fragment and primers HChuF2 and HChuR2 for the C-terminal fragment. The PCR resulted in a variable domain fragment containing two additional mutations, N87 to S and M95 to V into FR3. The cloning of this fragment into pGEM T-Easy resulted in the construct pJRS364.

3. Site directed mutagenesis using the QuikChange system (Stratagene) was then used to introduce another mutation into the heavy chain variable domain. A pair of complementary primers JSS80 and JSS81 were combined with plasmid pJRS364 and amplfied with PfuTurbo DNA polymerase. Following digestion of the products with DpnI, the DNA was used to transform E. coli, XL1B cells. This manipulation resulted in the V99 to A mutation in FR3 and the construct was designated pJRS373. The cloning of this fragment into the expression vector pJRS355 as a BsiWI to EcoRI restriction fragment resulted in plasmid pJRS380.

4. Site directed mutagenesis using the QuikChange system (Stratagene) was then used to introduce another mutation into the heavy chain variable domain. A pair of complementary primers JSS82 and JSS83 were combined with plasmid pJRS373 and amplfied with PfuTurbo DNA polymerase. Following digestion of the products, the DNA was used to transform E. coli, XL1B cells. This manipulation resulted in the K15 to G mutation in FR1 and the construct was designated pJRS378. The cloning of this fragment into the expression vector pJRS355 as a BsiWI to EcoRI restriction fragment resulted in plasmid pJRS381.

5. PCR amplification of anti-LTA mAb A110 heavy chain (HC) variable domain fragments was performed using plasmid pJRS381 (an expression vector for a mutated HCV of A110) as template. Primers MV-HC Leader and JSS87, for the N-terminal fragment, and primers JSS86 and HCV Back, for the C-terminal fragment. The PCR resulted in a variable domain fragment containing three additional mutations, Q78 to K, S79 to N, and, M80 to S into FR3. The cloning of this fragment into pJRS355 as a BsiWI to EcoRI restriction fragment resulted in the construct pJRS383.

Primers Used for Heavy Chain Humanization

D. Generation of Humanized Anti-LTA Antibody Light Chain

1. PCR amplification and cloning into pGEM T-easy (Promega) of anti-LTA mAb A110 heavy chain (LC) variable domain were performed using plasmid pJRS334 (an expression vector for A110) as template and primers LChuF1 and LChuR3. Primer LChuF1 introduced a AgeI site upstream of the first codon of the variable domain and also amino acid changes, D1 to Q and S5 to T, in framework 1. Primer LChuR3 introduced an amino acid change M102 to L in FR4 and a C-terminal BstBI restriction site for cloning purposes. This step resulted in the construct pJRS363.

2. PCR amplification of anti-LTA mAb A110 light chain (LC) variable domain fragments was performed using plasmid pJRS334 (an expression vector for A110) as template and primers LChuF1 and LChuR1 for one N-terminal fragment (LCN1) and primers LChuF1 and LChuR2 for a second N-terminal fragment (LCN2). Two C-terminal fragments were generated using the primers LChuF2 and LChuR3 (LCC1) for one, and LChuF3 and LChuR3 for the second (LCC2). An internal fragment for PCR was also generated using the primers LChuF2 and LChuR2 (LCI).

3. PCR reactions using fragments LCN1 and LCC1 as templates and LChuF1 and LChuR3 as primers were performed and the resulting products were cloned into pGEM T-easy to produce the plasmid pJRS365. This introduced another mutation, S41 to Q in FR2.

4. PCR reactions using fragments LCN2 and LCC2 as templates and LChuF1 and LChuR3 as primers were performed and the resulting products were cloned into pGEM T-easy to produce the plasmid pJRS366. This introduced two other mutations, R76 to A and V77 to M in FR3.

5. PCR reactions using fragments LCI and LCC2 as templates and LChuF1 and LChuR3 as primers were performed. A second PCR was then done using this new fragment and LCN1 as template and LChuF1 and LChuR3 as primers. The resulting product was cloned into pGEM T-easy to produce the plasmid pJRS367. This combined all of the light chain mutations into one light chain variable domain fragment.

6. The L102 in all of the LC variable domain clones was then converted to a K by PCR. Using plasmids pJRS363, 365, 366 and 367 as templates, the variable domains were re-amplified using primers LChuF1 and LChuR4. The resulting products were again cloned into pGEM T-easy to generate plasmids pJRS363K, 365K, 366K, and 367K.

7. All four versions of humanized variable domain were subsequently cloned into a light chain expression vector, tKMC180, as an AgeI to BstBI restriction fragment. The final constructs for mammalian cell expression of the various mutant light chains were designated pJRS374, 375, 376, and, 377.

8. PCR amplification of anti-LTA mAb A 10 light chain (LC) variable domain fragments was performed using plasmid pJRS376 (an expression vector for humanized A110 light chain containing 6 mutations) as template and primers MV-LC leader and JSS89 for the N-terminal fragment (LCN1) and primers JSS90 and LC reverse for the C-terminal fragments. PCR reactions using these fragments as template and primers MV-LC leader and LC reverse resulted in products containing the mutations L11 to M and M21 to I. These products were then cloned into tKMC180, the LC expression vector, as an AgeI to BstBI restriction fragment to generate the plasmid pJRS384.

All of the above expression vector constructs were tested positive for the ability to express antibodies in co-transfection experiments using COS cells. All of the combinations of humanized heavy and light chain A110 variable domains bound to LTA when tested using ELISA. Subsequently, combination expression vectors, that is, single plasmids with both heavy and light chain coding regions, were constructed. The final expression plasmid containing all 16 mutations (8 heavy and 8 light) made from the variable domains cut from pJRS383 and pJRS384 was designated pJRS394. When this plasmid was used to transfect COS cells, the resulting antibody produced was able to bind to LTA as shown by ELISA experiments.

Primers Used for Cloning:

| | |
|---|---|
| Name | Oligo Sequence |

| | |
|---|---|
| HC-MV Leader | GGAGACCCAAGCTTGTTAAC |
| HCV back | CCCAGAGGTGCTCTTGGAG |
| HChuF1 | TGTTTTCGTACGTCTTGTCCGAAGTGCAGCTGGTGGAGTCTG |
| HChuF2 | GAACAGCTTGAAAACTGAGGACACAGCCGTGTATTACTGTGTGAGAC |
| HChuR1 | AATACACGGCTGTGTCCTCAGTTTTCAAGCTGTTCATTTGCAGATAGAGCATG |
| HChuR2 | AATTCTGAATTCTGAGGAGACGGTCACTGAGGTTCCTTGACCCC |
| JSS80 | CAGCCGTGTATTACTGTGCGAGACGGGGGGCTTC |
| JSS81 | GAAGCCCCCCGTCTCGCACAGTAATACACGGCTG |
| JSS82 | GGATTGGTGCAGCCTGGCGGGTCATTGAAACTCTC |
| JSS83 | GAGAGTTTCAATGACCCGCCAGGCTGCACCAATCC |
| JSS84 | GAGGCTCTGCACAACCGCTTCACGCAGAAGAGCCTCTCC |
| JSS85 | GGAGAGGCTCTTCTGCGTGAAGCGGTTGTGCAGAGCCTC |
| JSS86 | GATTCAAAAAACAGCCTCTATCTGCAAATGAACAACTTG |
| JSS87 | CAGATAGAGGCTGTTTTTTGAATCATCTCTGGAGATGG |
| LC-MV Leader | GAGACCCAAGCTTGGTACC |
| LC reverse | CTGACTTTAACTCCTAACATG |
| JSS88 | AATCATGTCTGCATCTCCAGGGGAAAAGGTCACAATCACTTGCAGGGCCAGCTC |
| JSS90 | CAAGTGATTGTGACCTTTTCCCCTGGAGATGCAGACATGATTGCTGGAGACTGGGAG |
| LChuF1 | ACTTATACCGGTCAGATCGTTCTCACCCAGTCTCCAGCAATC |
| LChuF2 | ACCAGCAGAAGCCAGGATCCCAGCCCAAACCCTGGATTTCTG |
| LChuF3 | CAGCGCAATGGAGGCTGAAGATGCTGCC |
| LChuR1 | TTGGGCTGGGATCCTGGCTTCTGCTGG |
| LChuR2 | CTTCAGCCTCCATTGCGCTGATTGTGAGAGAGTAAG |
| LChuR3 | ATTACCTTCGAAAAGTGTACTTACGTTTTATTTCCAGGTTGGTCCCCCCTCCGAAC |
| LChuR4 | ATTACCTTCGAAAAGTGTACTTACGTTTTATTTCCAGCTTGGTCCCCCCTCCGAAC |

### EXAMPLE 5

### Characterization of Humanized Anti-LTA Antibodies

A. Transient Production of Recombinant Humanized c96-110 Antibody Variants

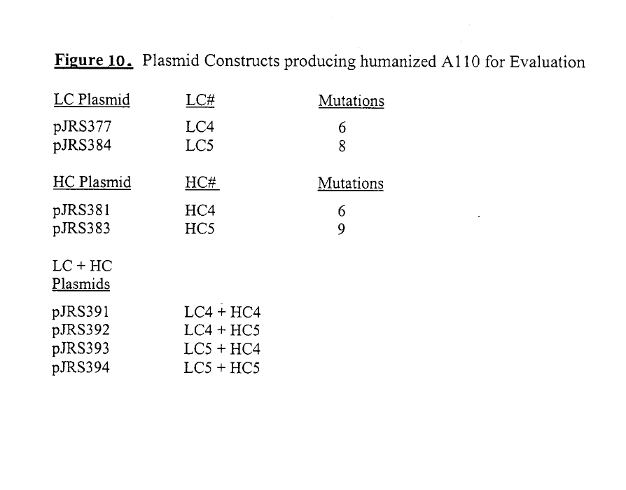

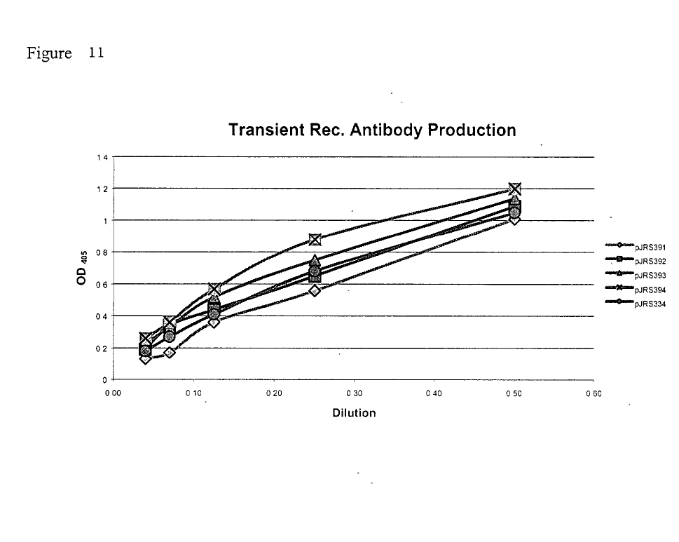

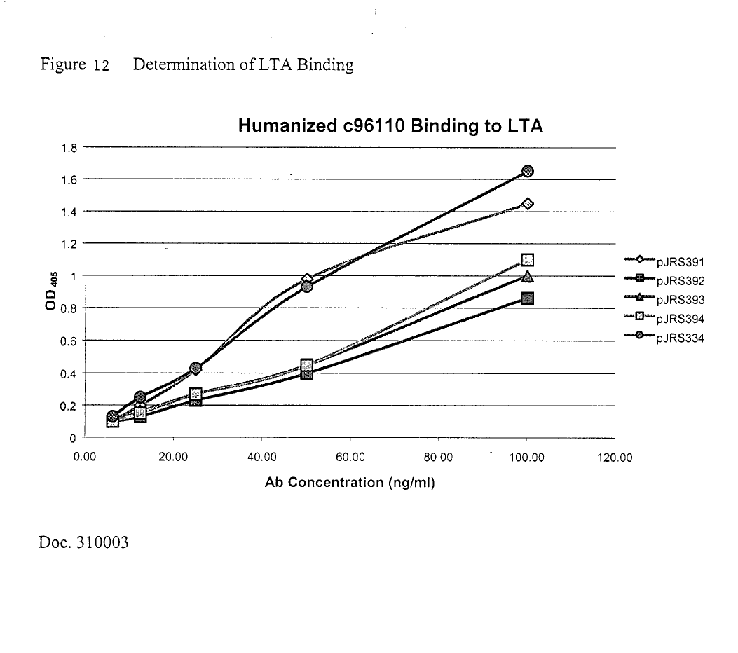

To characterize the humanized versions of the anti-LTA antibody, the plasmids pJRS334, 391, 392, 393, and 394 (see FIG. 10) were transected into COS cells using Superfect (Qiagen) in 6 well tissue culture wells as described by the manufacturer. The plasmid pJRS334 encodes the c96-110 antibody and the plasmids pJRS391-4 encode humanized variants of c96-110. After two days the supernatant was assayed for the production of chimeric antibody. These antibodies were then assayed for the capability for the expressed antibody to bind to S. aureus LTA antigen.

Antibody production assays were preformed in 8-well strips from 96-well microtiter plates (Maxisorp F8; Nunc, Inc.) coated at a 1:500 dilution with a goat antihuman Fc (Pierce). The plates are covered with pressure sensitive film and incubated overnight at 4° C. Plates were then washed once with Wash solution (Imidazole/NaCl/0.4%Tween-20). One hundred microliters of culture supernatant dilutions of the transiently transfected COS cells were then applied to duplicate wells and allowed to incubate for 60 minutes on plate rotator at room temperature. The plates were washed seven times with Wash solution. A Goat anti-Human IgG H+L-HRP (Zymed) conjugate was diluted 1:4000 in the sample/conjugate diluent and one hundred microliters of the dilution was added to each of the samples, and then incubated on a plate rotator for 60 minutes at room temperature. The samples were washed as above and then incubated with 100 μL/well of ABTS developing substrate (BioFx) for 1 minute at room temperature. The reaction was stopped with 100 μL/well of Quench buffer (BioFx) and the absorbance value at 405 nm was determined using an automated microtiter plate ELISA reader (see results in Table 8 and FIG. 11). This assay demonstrates that the transfections of COS cells with these plasmid constructs results in the cells producing molecules containing both human IgG and Kappa domains. Approximation of antibody concentration in each of the cellular supernatants was determined by comparisons to a standard curve dilution series using human monoclonal IgG1 at 0.5 μg/mL to 0.04 μg/mL.

TABLE 8
Antibody Production in COS Cells (O.D._{405 nm})

| Dilution | pJRS391 | pJRS392 | pJRS393 | pJRS394 | pJRS334 |
|---|---|---|---|---|---|
| 1:1 | 1.01 | 1.09 | 1.14 | 1.2 | 1.05 |
| 1:2 | 0.56 | 0.65 | 0.75 | 0.88 | 0.68 |
| 1:4 | 0.36 | 0.44 | 0.52 | 0.57 | 0.41 |
| 1:8 | 0.17 | 0.34 | 0.33 | 0.36 | 0.27 |
| 1:16 | 0.13 | 0.19 | 0.23 | 0.26 | 0.18 |

B. Characterization of the Antibody Binding to LTA by the Humanized Variants.

The antibody containing culture supernatants from the transiently transfected COS cells were then assayed for the ability of the expressed antibodies to bind to S. aureus LTA. The activity assays were preformed in 8-well strips from 96-well microtiter plates (Maxisorp F8; Nunc, Inc.) coated at 1 μg/mL with S. aureus LTA (Sigma) using PBS. The plates were covered and incubated overnight at 4° C. Plates are then washed once with PBS. One hundred microliters of culture supernatant dilutions were then applied to duplicate wells and allowed to incubate for 60 minutes on a plate rotator at room temperature. The plates were washed seven times with Wash solution. The goat anti-Human IgG H+L-HRP (Zymed) was diluted 1:4000 in the sample/conjugate diluent and one hundred microliters of the dilution was added to each of the samples, and then incubated on a plate rotator for 60 minutes at room temperature. The samples were washed as above and then incubated with 100 μL/well of ABTS developing substrate (BioFx) for 10-15 minutes on a plate rotator at room temperature. The reaction was stopped with 100 μL/well of Quench buffer (BioFx) and the absorbance value at 405 nm was determined using an automated microtiter plate ELISA reader (see results in Table 9 and FIG. 1). This assay demonstrates that the transfection of cells with these plasmid constructs result in the cells producing comparable levels of humanized A110 antibodies.

TABLE 9
Antibody Binding Assay (O.D._{405 nm})

| equivalent | | | | | |
|---|---|---|---|---|---|
| [Ab] | | | | | |
| in ng/mL | pJRS391 | pJRS392 | pJRS393 | pJRS394 | pJRS334 |
| 100.00 | 1.45 | 0.86 | 1 | 1.1 | 1.65 |
| 50.00 | 0.98 | 0.4 | 0.45 | 0.45 | 0.93 |
| 25.00 | 0.42 | 0.23 | 0.27 | 0.27 | 0.43 |
| 12.50 | 0.2 | 0.13 | 0.16 | 0.15 | 0.25 |
| 6.25 | 0.1 | 0.1 | 0.1 | 0.1 | 0.13 |

C. Affinity Constant Determinations of c96-110 Using Surface Plasmon Resonance (SPR) Analysis.

These antibodies were subsequently examined using surface plasmon resonance to evaluate antibody binding to the S. aureus LTA antigen. It is apparent from these results in Table 10 that the humanized A110 antibody produced by pJRS391 is comparable in its ability to bind LTA to the parent antibody A110 (or c96-110). Therefore the humanized version must have a similar apparent binding affinity to LTA as its parent.

C-1. Vesicle Preparation and Immobilization.

Lipoteichoic acid (LTA) containing vesicles were prepared according to the method of Kalb et al., Biochemica et Biophysica Acta (1992) 1103, 307-316. Briefly, phosphatidyl-ethanolamine linoleoyl-palmitoyl (PE-L-P, SIGMA, St. Louis, Mo.) solution in chloroform is evaporated to dryness under vacuum. BIAcore eluent (HBS) and LTA from Staphylococcus aureus (SIGMA, St. Louis Mo.) was added to make a 0.2 mM PE-L-P solution in HBS and 1% LTA in PE-L-P. After vigorous vortexing the solution is passed 8 times through a polycarbonate filter (0.1 μm pores, Nucleopore Coming). This solution is immediately injected onto the BIAcore HPA chip (BIAcore Inc. Piscataway, N.J.) until a plateau of approx. 1400 RU is obtained. The vesicles spontaneously fuse to the surface of a HPA chip.

C-2. SPR Analysis

The binding kinetics were determined on a BIAcore instrument (BIAcore Inc., Piscataway, N.J.) fitted with a HPA chip coated with PE-L-P/1% LTA. Different concentrations of c96-110 were injected over the surface. Since the chip surface could not be regenerated, only 1 injection per surface was performed. The association and dissociation rates were determined with the BIAevaluation Software 2.0 (BIAcore Inc., Piscataway, N.J.) using the one to one binding model.

TABLE 10
Apparent Affinity constants for c96-110 for different
concentrations of LTA

| LTA conc. | kₐ [M⁻¹ s⁻¹] | k_{d} [s⁻¹] | K_{A} [M⁻¹] |
|---|---|---|---|
| 0.1% | 1.68 × 10⁶ | 1.68 × 10⁻² | 1 × 10⁸ |
| 1% | 1.33 × 10⁶ | 115 × 10⁻² | 1.16 × 10⁸ |
| 10% | 2.07 × 10⁵ | 3.51 × 10⁻⁴ | 5.89 × 10⁸ |

### EXAMPLE 6

### Comparison in Silico of Humanization of Anti-TAC Antibody with the FR Best Fit Approach of the Invention

To compare approaches, specific antibodies for which humanization has been performed and described in publications are compared to the results of humanization by the FR best fit approach. When the original humanization work was performed in the past, the Kabat database was not as extensive as it was when this in silico comparison was performed. The expansion of the database provides an increased number of frameworks that could be considered for best fit. To correct for the bias introduced as a result of the expanded database, a revised best fit search was performed and the results presented.

In this Example, the comparison was performed for the anti-TAC antibody. The in silico humanization is presented in Tables 11 and 12. In the first line of each table, the starting murine antibody sequence is reported and the second line is the sequence of the original humanized antibody (Queen, et al. PNAS 86: 10029-10033 (1989)) where the framework showing the best fit was 035921 for the light chain variable domain and 035918 for the heavy chain variable domain. The third line in each table shows the preferred best fit framework as determined at the time of this comparison. The last line shows the sequence that would be preferred using the FR best fit approach.

In performing this comparison, the benefits of the FR best fit approach can be seen by reviewing the overall number of amino acid substitutions required, the number of vernier residues that required changing, and the overall homology score. By minimizing the number of amino acid substitutions, the time, cost and labor involved in the actual humanization are reduced. By identifying FRs in which the vernier residues are maintained as the preferred amino acid, deleterious effects on the confirmation of the CDRs are minimized which should lead to minimal effects on antibody binding affinity. A better overall homology score (i.e. % homology) for the light and heavy chain frameworks is seen for the FR best fit approach compared to the other framework based approaches.

For the anti-TAC antibody, the original humanization was based on the selection of 035921 and 035918 as the frameworks with the best fit for the light chain and the heavy chain respectively. This humanization required substitution of 28 (five of which are vernier residues) of the 86 amino acids in the light chain framework and 29 (five of which are vernier residues) of 87 amino acids in the heavy chain framework. Using the expanded Kabat database, the CEA4-8A (004752) framework had the best fit for the light chain and the A110 (045903) antibody had the best fit for the heavy chain (using relaxed criteria which assumes that the light and heavy chain variable domain frameworks do not have to come from the same antibody). This resulted in a better fit compared to the original humanized anti-TAC, and would require fewer total substitutions (30 vs. 57) and fewer changes in vernier zone residues (4 vs. 10) and better homology. But the FR best fit gives the superior result, requiring only 23 amino acid substitutions, of which only 3 are vernier residues. The overall homology between the murine anti-TAC and the original humanized anti-TAC was 74.3%. The overall homology improves to 86.5% with the expanded database but improves to 89.6% when the FR best fit approach is applied.

| | | |
|---|---|---|
| aa Changes | Vernier | % Homology |

Comparison of Humanization Approaches for Anti-TAC Light Chain

| | | | |
|---|---|---|---|
| huAnti-TAC | 28/106 | 5 | 73.6% |
| CEA4-8A (004752) | 24/106 | 3 | 77.4% |
| FR Best Fit | 18/106 | 2 | 83% |

Comparison of Humanization Approaches for Anti-TAC Heavy Chain

| | | | |
|---|---|---|---|
| huAnti-TAC | 29/116 | 5 | 75% |
| A10 (045903) | 6/116 | 1 | 94.8% |
| FR Best Fit | 5/116 | 1 | 95.7% |

Overall Comparison of Humanization Approaches for
Anti-TAC Variable Domains

| | | | |
|---|---|---|---|
| huAnti-TAC | 57/222 | 10 | 74.3% |
| A10 (045903) | 30/222 | 4 | 86.5% |
| FR Best Fit | 23/222 | 3 | 89.6% |

TABLE 11

Comparison of humanized anti-TAC Light Chain by approaches
including the FR Best Fit Approach
Table 11A

| Names | LC-FR1 (23 aa) | LC-FR2 (15 aa) |
|---|---|---|
| | 1 10 20 | 35 49 |
| muAnti-TAC | QIVLTQSPAIMSASPGEKVTITC | WFQQKPGTSPKLWIY |
| huAnti-TAC | D QM STL V DR | Y KA LM |
| CEA4-8A | E SSL V DR | Y KA L |
| | 004752 | |
| FR Best Fit | E TL V DR M | KA L |
| | ABO.B1 (047269) | G3D10K (005121) |

Table 11B

| Names | LC-FR3 (32 aa) | LC-FR4 (10 aa) |
|---|---|---|
| | 57 60 70 80 | 98 107 |
| muAnti-TAC | GVPARFSGSGSGTSYSLTISRMEAEDAATYYC | FGSGTKLELK |
| huAnti-TAC | S I EFT SLQPD F | Q V V |
| CEA4-8A | S DFT SLQP F | G VDI |
| | 004752 | |
| FR Best Fit | D SF SLQ V | Q |
| | FRA (037670) | HSC13 (036038) |

TABLE 12

Comparison of humanized anti-TAC Heavy Chain by approaches
including the FR Best Fit Approach
Table 12A

| Names | HC-FR1 (30 aa) | HC-FR2 (14 aa) |
|---|---|---|
| | 1 10 20 30 | 36 49 |
| muAnti-TAC | QVQLQQSGAELAKPGASVKMSCKASGYTFT | WVKQRPGQGLEWIG |
| huAnti-TAC | V VK S V G S | R A M |
| A10 (045903) | V V D | Unchanged |
| FR Best Fit | V V D | Unchanged |
| | A10 | A10 |

Table 12B

| Names | HC-FR3 (32 aa) | HC-FR4 (11 aa) |
|---|---|---|
| | 67 75 85 95 | 107 117 |
| muAnti-TAC | KATLTADKSSSTAYMQLSSLTFEDSAVYYCAR | WGQGTTLTVSS |
| huAnti-TAC | RV I E TN E RS T F F G | EYN GLV |
| A10 (045903) | V S | V |
| FR Best Fit | V S | Unchanged |
| | A10 (045903) | SCF11 (041948) |

### EXAMPLE 7

### Comparison in Silico of Humanization of the Mc3 Antibody with the FR Best Fit Approach of the Invention

As in the previous example, a second humanized antibody is re-examined in silico in this example to compare the humanization approach used for this antibody to humanization by the FR best fit approach. A revised best fit search was performed and the results presented in order to correct for the bias introduced as a result of the expanded database.

In this Example, the comparison was performed for the Mc3 antibody. The in silico humanization is presented in Tables 13 and 14. In the first line of each table, the starting murine antibody sequence is reported and the second line is the sequence of the original humanized antibody (U.S. Pat. No. 5,639,641). The third line in each table shows the preferred best fit framework as determined at the time of this comparison. The last line shows the sequence that would be preferred using the FR best fit approach.

For the Mc3 antibody, the original humanization was disclosed in U.S. Pat. No. 5,639,641. This humanization required substitution of 16 residues (none of which were vernier residues) of the 107 amino acids in the light chain framework and 14 residues (none of which were vernier residues) of 117 amino acids in the heavy chain framework. Using the expanded Kabat database, the VL Clone 47 (024300) framework had the best fit for the light chain variable domain and the A110 (045903) antibody had the best fit for the heavy chain variable domain (using relaxed criteria which assumes that the light and heavy chain variable domain frameworks do not have to come from the same antibody). Even using these frameworks, the resulting in silico “humanized” antibody is not as good as the originally humanized antibody. This is due to the fact that additional changes were recommended by the method originally for the original humanized Mc3. The best fit framework from the in silico humanization would require 35 amino acid substitutions, compared to the 30 amino acid changes that were required by the original humanization. No changes in vernier zone residues are required in either humanization. But invention's FR best fit approach gives the superior result, requiring only 26 amino acid substitutions, and no vernier residues changes. The overall homology between the murine Mc3 and the in silico humanized example is 82.2% and 85% for the original humanized Mc3. The best overall is 89.7% for present FR best fit approach.

| | | |
|---|---|---|
| aa Changes | Vernier | % Homology |

Comparison of Humanization Approaches for Mc3 Light Chain

| | | | |
|---|---|---|---|
| huMc3 (037000) | 16/107 | 0 | 85% |
| Framework (024300) | 19/107 | 0 | 82.2% |
| FR Best Fit | 11/107 | 0 | 89.7% |

Comparison of Humanization Approaches for Mc3 Heavy Chain

| | | | |
|---|---|---|---|
| huMc3 (037000) | 14/117 | 0 | 88% |
| Framework (045903) | 16/117 | 0 | 86.3% |
| FR Best Fit | 15/117 | 0 | 87.2% |

Overall Comparison of Humanization Approaches
for Mc3 Variable Domains

| | | | |
|---|---|---|---|
| huMc3 (037000) | 30/224 | 0 | 86.6% |
| Framework | 35/224 | 0 | 84.4% |
| FR Best Fit | 26/224 | 0 | 88.4% |

TABLE 13

Comparison of humanized Mc3 Light Chain using Immunogen's
Templates vs. Framework Best Fit vs. FR Best Fit Approach
Table 13A

| Names | LC-FR1 (23 aa) | LC-FR2 (15 aa) |
|---|---|---|
| | 1 10 20 | 35 49 |
| Mc3 -LC | DIVMTQSHKFMSTSEGDWVSITC | WYQQKPGQSPKLLIY |
| HuMc3-LC | PDSLAV L ERAT | unchanged |
| | 037000 | 037000 |
| Framework | PSSL A V R T | KA |
| | 024300 | |
| FR Best Fit | PSSL A V R | unchanged |
| | 047664 | 047246 |

Table 13B

| Names | LC-FR3 (32 aa) | LC-FR4 (10 aa) |
|---|---|---|
| | 57 60 70 80 88 | 98 107 |
| Mc3 -LC | GVPDRFSGSGSGTDFTFTISSVQAEDLAVYYC | FGSGTNLEIK |
| HuMc3-LC | L L V | KV |
| | 037000 | 037000 |
| Framework | S L L P F T | G KV |
| | 024300 | |
| FR Best Fit | L L V | Q |
| | 005062 | 047666 |

TABLE 14

Comparison of humanized Mc3 Heavy Chain using Immunogen's
Templates vs. Framework Best Fit vs. FR Best Fit Approach

TABLE 14A

| Names | HC-FR1 (30 aa) | HC-FR2 (14 aa) |
|---|---|---|
| Mc3-HC | 1 10 20 30 | 36 44 |
| | EVQLQQSGPELVKPGASMKISCEASGYSFT | WVKQSHGMNLEWIG |
| HuMc3-HC | V A VK V V K | P |
| | 037010 | |
| Framework | Q A V L K D T | RP QG |
| | 045903 | |
| FR Best Fit | V A VK V V K | RP QG |
| | 050357 | 045903 |

TABLE 14B

| Names | HC-FR3 (32 aa) | HC-FR4 (11 aa) |
|---|---|---|
| Mc3-HC | 67 75 85 95 | 107 117 |
| | KATLTVDKSSGTAYMELLSLTSEDSAVYFCAR | WGQGTSVTVSS |
| HuMc3-HC | TS S R T | L |
| | 037010 | |
| Framework | S Q S Y | T |
| | 045903 | |
| FR Best Fit | S Q S Y | unchanged |
| | 045903 | 041935 |

### EXAMPLE 8

### Comparison in Silico of Humanization of Anti-TF Antibody: Framework vs. FR Best Fit Approach

Another comparison of humanization methods can be made using the antibody from Example 1 described above. A comparison is made for the best fit by entire framework approach and compared to the FR best fit approach for the anti-TF antibody. Again this comparison was performed in silico more recently compared to the original humanization and the resulting best fit is not always identical to the results presented in Example 1 due to the expansion of the Kabat database providing FRs with better fits. Nonetheless, the conclusion remains that the FR best fit approach provides advantages over the framework best fit approaches.

When the search is performed to identify the best fit framework, the results are as shown below in Tables 15 and 16. The first line in the table is the original murine monoclonal antibody sequence, the second line shows the original FR best fit sequence for the humanized anti-TF antibody from Example 1, the third line is the sequence for the best fit framework and the fourth line is the best fit determined more recently using the FR best fit approach. The best fit framework for the anti-TF antibody light chain (Table 15) is scF11 (041950) which would require 13 amino acids substitutions, changing 1 vernier zone residues and a homology of 87.9%. The updated FR best fits for the anti-TF antibody light chain are 041950 for FR1, 019308 for FR2, 038233 for FR3, and 036038 for FR4 which would require 10 amino acids substitutions, changing 0 vernier zone residues and a homology of 90.7%. The best fit framework for the anti-TF antibody heavy chain (Table 16) is A10 (045903) which would require 20 amino acids substitutions, changing 1 vernier zone residues and a homology of 82.9%. The FR best fit approach for the anti-TF antibody heavy chain is 000042 for FR1, 023960 for FR2, 045903 for FR3, and 047722 for FR4 which would require 15 amino acids substitutions, changing 0 vernier zone residues and a homology of 87.2%. Overall the FR best fit approach requires 25 amino acids substitutions, changing no vernier zone residues and a homology of 88.8% compared to the framework best fit approach which would require 33 amino acids substitutions, changing 2 vernier zone residues and a homology of 85.3%.

Overall Comparison of Humanization Approaches for
Anti-TF Variable Domains

| | aa Changes | Vernier | % Homology |
|---|---|---|---|
| Example 1 | 30/224 | 0 | 86.6 |
| Framework | 33/224 | 2 | 85.3 |
| FR Best Fit | 25/224 | 0 | 88.8 |

TABLE 15

Comparison of humanized anti-TF Light Chain using
Framework Best Fit vs. FR Best Fit Approach

TABLE 15A

| Names | LC-FR1 (23 aa) | LC-FR2 (15 aa) |
|---|---|---|
| cH36- | 1 10 20 | 35 49 |
| LC | DIQMTQSPASQSASLGESVTITC | WYQQKPGKSPQLLIY |
| Human- | L V DR | L |
| LC | 005191 | 019308 |
| Framework | L V T | Q V |
| | 041950 | |
| FR | L V T | L |
| Best | 041950 | 019308 |
| Fit | | |

TABLE 15B

| | | LC-FR4 |
|---|---|---|
| Names | LC-FR3 (32 aa) | (10 aa) |
| cH36- | 57 60 70 80 88 | 98 107 |
| LC | GVPSRFSGSGSGTKFSFKISSLQAEDFVNYYC | FGAGTKLELK |
| Human- | D T P AT | Q I |
| LC | 038233 | 004733 |
| Frame- | Q L N P GS | S I |
| work | 041950 | |
| FR | D T P AT | Q |
| Best | 038233 | 036038 |
| Fit | | |

TABLE 16

Comparison of humanized anti-TF Heavy Chain using
Framework Best Fit vs. FR Best Fit Approach

TABLE 16A

| Names | HC-FR1 (30 aa) | HC-FR2 (14 aa) |
|---|---|---|
| cH36-HC | 1 10 20 30 | 36 49 |
| | EIQLQQSGPELVKPGASVQVSCKTSGYSFT | WVRQSHGKSLEWIG |
| Human-HC | Q V GVK R A | P G |
| | 000042 | 023960 |
| Framework | QV A KL A D T | K RP QG |
| | A10 (045903) | |
| FR Best Fit | Q V G VK R A | P G |
| | 000042 | 023960 |

TABLE 16B

| Names | HC-FR3 (32 aa) | HC-FR4 (11 aa) |
|---|---|---|
| cH36-HC | 67 75 85 95 | 107 117 |
| | KATLTVDKSSTTAFMHLNSLTSDDSAVYFCAR | WGQGTTLTVSS |
| Human-HC | TS Y E S R E T | V |
| | 037010 | 000049 |
| Framework | S Y Q S E Y | V |
| | A10 (045903) | |
| FR Best Fit | S Y Q S E Y | unchanged |
| | A10 (045903) | 047722 |

### EXAMPLE 9

### Comparison in Silico of Humanization of Anti-LTA Antibody: Framework vs. FR Best Fit Approach

Another comparison of humanization methods can be made using the antibody from Example 3 described above. A comparison is made for the best fit by entire framework approach and compared to the FR best fit approach for the anti-LTA antibody (A110). As in Example 8, this comparison was performed in silico more recently compared to the original humanization and the resulting best fit might not have been identical to the results presented in Example 3 due to the expansion of the Kabat database providing FRs with better fits. In this particular case the FRs with the best fit are identical in both the earlier humanization and in this more recent in silico comparison. However the results support the conclusion that the FR best fit approach provides advantages over the framework best fit approaches.

When the search is performed to identify the best fit framework, the results are as shown below in Tables 17 and 18. The first line in the table is the original murine monoclonal antibody sequence, the second line shows the original FR best fit sequence for the humanized anti-LTA antibody from Example 4, which turns out to be the same result when the FR best fit search was conducted more recently, and the third line is the sequence for the best fit framework determined recently. The best fit framework for the anti-LTA antibody light chain variable domain (Table 17) is 036047 which would require 13 amino acids substitutions out of 107 amino acids, changing 2 vernier zone residues and a homology of 87.9%. The FR best fits for the anti-LTA antibody light chain are 036047 for FR1, 037658 for FR2, 036047 for FR3, and 004763 for FR4 which would require 9 amino acids substitutions, changing no vernier zone residues and a homology of 91.6%. The best fit framework for the anti-LTA antibody heavy chain variable domain (Table 18) is 028897 which would require 15 amino acids substitutions out of 123 amino acids, changing 4 vernier zone residues and a homology of 87.8%. The FR best fit approach for the anti-TF antibody heavy chain is 000468 for FR1, 000565 for FR2, 000628 for FR3, and 031571 for FR4 which would require 9 amino acids substitutions, changing 2 vernier zone residues and a homology of 92.7%. Overall the FR best fit approach requires 18 amino acids substitutions, changing 2 vernier zone residues and a homology of 92.2% compared to the framework best fit approach which would require 28 amino acids substitutions, changing 6 vernier zone residues and a homology of 87.8%.

TABLE 17

Comparison of humanized anti-LTA Light Chain using
Framework Best Fit vs. FR Best Fit Approach

TABLE 17A

| Names | LC-FR1 (23 aa) | LC-FR2 (15 aa) |
|---|---|---|
| A110-LC | 1 10 20 | 35 49 |
| | DIVLSQSPAILSASPGEKVTMTC | WYQQKPGSSPKPWIS |
| Human-LC | Q T M D I | Q |
| | 036047 | 037658 |
| Framework | Q T M D I | F T L Y |
| | 036047 | |

TABLE 17B

| Names | LC-FR3 (32 aa) | LC-FR4 (10 aa) |
|---|---|---|
| A110-LC | 57 60 70 80 88 | 98 107 |
| | GVPARFSGSGSGTSYSLTISRVEAEDAATYYC | FGGGTMLEIK |
| Human-LC | AM | K |
| | 036047 | 004763 |
| Framework | AM | S K |
| | 036047 | |

TABLE 18

Comparison of humanized anti-LTA Heavy Chain using
Framework Best Fit vs. FR Best Fit Approach

TABLE 18A

| Names | HC-FR1 (25 aa) | HC-FR2 (14 aa) |
|---|---|---|
| A110-HC | 1 10 20 | 36 49 |
| | EVMLVESGGGLVQPKGSLKLSCAAS | WVRQAPGKGLEWVA |
| Human-HC | Q G | unchanged |
| | 000468 | 000565 |
| Framework | Q K R R | G |
| | 028897 | |

TABLE 18B

| | | HC-FR4 |
|---|---|---|
| Names | HC-FR3 (32 aa) | (11 aa) |
| A110- | 67 75 85 95 | 107 117 |
| HC | RFTISRDDSQSMLYLQMNNLKTEDTAMYYCVR | WGQGTSLTVSS |
| Human- | KNS S V A | V |
| HC | 000628 | 031571 |
| Frame- | KNT S V TT | G LV |
| work | 028897 | |

The invention has been described in detail with reference to preferred embodiments thereof. However, it will be appreciated that those skilled in the art, upon consideration of the disclosure, may make modification and improvements within the spirit and scope of the invention.

What is claimed is:

1. A method for producing a humanized antibody variable (V) domain or a fragment thereof, wherein the method comprises:
a) comparing the amino acid sequence of a framework region (FR) of a non-human antibody variable (V) domain to a collection of human antibody framework amino acid sequences, or fragments thereof,
b) selecting a human FR sequence from the collection having the greatest amino acid sequence identity to the non-human FR,
c) mutagenizing DNA of the non-human FR to encode a humanized FR (huFR) having an amino acid sequence substantially identical to the selected human FR from step b),
d) repeating steps a) thru c) for each of the FRs in the non-human V domain to produce a plurality of DNA sequences in which each DNA sequence encodes a humanized FR; and
e) substituting into a first vector encoding at least the V domain of the non-human antibody, each of the huFR DNA sequences from step d) for the corresponding non-human FRs encoded by the vector; wherein the substitution operatively links each of the huFRs to its corresponding complimentarity determining region (CDR); and
f) expressing the first vector in host cells and under conditions conducive to making the humanized antibody V domain or the fragment thereof.

2. The method of claim 1, wherein the V domain or fragment is from a non-human antibody light chain.

3. The method of claim 2, wherein the framework region (FR) of the light chain V domain of step a) is FR1.

4. The method of claim 3, wherein sequence identity between the FR1 of the non-human antibody light chain and the selected human FR is at least about 70%.

5. The method of claim 4, wherein step d) further comprises comparing a second framework region (FR2) of the non-human light chain V domain to the collection and selecting a human FR having at least about 70% sequence identity.

6. The method of claim 5, wherein step d) further comprises comparing a third framework region (FR3) of the non-human light chain V domain to the collection and selecting a human FR having at least about 70%sequence identity.

7 The method of claim 6, wherein step d) further comprises comparing a fourth framework region (FR4) of the non-human light chain V domain to the collection and selecting a human FR having at least about 70% sequence identity.

8. The method of claim 7, wherein the humanized light chain V domain comprises covalently linked in sequence: huFR1-CDR1-huFR2-CDR2-huFR3-CDR3-huFR4; or a fragment thereof.

9. The method of claim 2-8, wherein the vernier zone amino acid residues in each FR are identical in the non-human and human FR of the antibody light chain V domain.

10. The method of claim 2, wherein the first vector further comprises a human light chain constant domain or fragment thereof covalently linked to the humanized light chain V domain.

11. The method of claim 10, wherein the human light chain constant domain is Cκ, Cγ or a fragment thereof.

11a. The method of claim 11, wherein the humanized light chain fragment has an amino acid length of between from about 95 to about 235 amino acids.

12. The method of claim 1, wherein the V domain or fragment is derived from non-human antibody heavy chain.

13. The method of claim 12, wherein the framework region (FR) of the heavy chain V domain of step a) is FR1.

14. The method of claim 13, wherein the sequence identity between the FR1 and the selected human framework FR is at least about 70%.

15. The method of claim 14, wherein step d) further comprises comparing a second framework region (FR2) of the non-human heavy chain V domain to the collection and selecting a human FR having at least about 70% sequence identity.

16. The method of claim 15, wherein step d) further comprises comparing a third framework region (FR3) of the non-human heavy chain V domain to the collection and selecting a human FR having at least about 70% sequence identity.

17 The method of claim 16, wherein step d) further comprises comparing a fourth framework region (FR4) of the non-human heavy chain V domain to the collection and selecting a human FR having at least about 70% sequence identity.

18. The method of claim 17, wherein the humanized light chain V domain comprises covalently linked in sequence: huFR1-CDR1-huFR2-CDR2-huFR3-CDR3-huFR4 or a fragment thereof.

19. The method of claim 12-18, wherein the vernier zone amino acid residues in each FR are identical in the non-human and human FR of the antibody heavy chain V domain.

20. The method of claim 12, wherein the first vector further comprises a human heavy chain constant domain or fragment thereof covalently linked to the humanized heavy chain V domain.

21. The method of claim 20, wherein the human heavy chain constant domain is one of an IgG1, IgG2, IgG3 or IgG4 isotype.

22. The method of claim 1, wherein the collection of human amino acid sequences comprises fully sequenced human antibodies.

23. The method of claim 22, wherein the collection further comprises amino acid sequences of partially sequenced human antibodies.

24. The method of claim 20, wherein the humanized heavy chain fragment has an amino acid length of between about 95 to about 540 amino acids.

25. A method for making a humanized antibody or a fragment thereof, wherein the method comprises:
a) comparing the amino acid sequence of a framework of a non-human antibody light chain variable (V) domain (1-FR) to a collection of human antibody light chain amino acid sequences, or fragments thereof,
b) selecting a human FR sequence from the collection having the greatest amino acid sequence identity to the 1-FR,
c) mutagenizing DNA of the 1-FR to encode a light chain humanized FR (L-huFR) having an amino acid sequence substantially identical to the selected human FR from step b),
d) repeating steps a) thru c) for each of the FRs in the light chain V domain to produce a plurality of DNA sequences in which each DNA sequence encodes an L-huFR,
e) substituting into a first vector encoding at least the light chain V domain of the non-human antibody, each of the L-huFR DNA sequences from step d) for the corresponding l-FRs encoded by the vector; wherein the substitution operatively links each of the L-huFRs to a corresponding complimentarily determining region (CDR),
f) comparing the amino acid sequence of a framework region of a non-human antibody heavy chain variable (V) domain (h-FR) to a collection of human antibody heavy chain amino acid sequences, or fragments thereof,
g) selecting a human FR sequence from the collection having the greatest amino acid sequence identity to the h-FR,
h) mutagenizing DNA of the h-FR to encode a humanized heavy chain FR(H-huFR) having an amino acid sequence substantially identical to the selected human FR from step g),
i) repeating steps f) thru h) for each of the h-FRs in the non-human heavy chain V region to produce a plurality of DNA sequences in which each DNA sequence encodes a H-huFR,
j) substituting into a second vector encoding at least the heavy chain V domain of the non-human antibody, each of the H-huFR DNA sequences from step i) for the corresponding h-FRs encoded by the vector; wherein the substitution operatively links each of the H-huFRs to a corresponding heavy chain CDR; and
k) expressing the first and second vectors in the same host cells and under conditions conducive to producing humanized light and heavy chains and making the humanized antibody or the fragment thereof.

26. The method of claim 25, wherein the DNAs encoding the humanized light and heavy chains or fragments thereof are contained on a single vector and co-expressed in the same host.

27. The methods of claims 25 and 26, wherein the host is mammalian, plant, avain or microbial.

28. The method of claim 25, wherein the first vector further comprises a human light chain constant domain or fragment thereof covalently linkedto the humanized light chain V domain.

29. The method of claim 28, wherein the light chain constant domain is Cic, Cy or a fragment thereof.

30. The method of claim 25, wherein the second vector further comprises a human heavy chain constant domain or fragment thereof covalently linked to the humanized heavy chain V domain.

31. The method of claim 30, wherein the human heavy chain constant domain is one of an IgG1, IgG2, IgG3 or IgG4 isotype.

32. The method of claim 25 or 26, wherein the method further comprises purifying the humanized antibody from the host cells to produce a substantially pure preparation of the antibody.

33. The method of claim 32, wherein the substantially purified humanized antibody specifically binds antigen with an affinity not less than about 10-fold lower than the parental non-human antibody.

34. The method of claim 33, wherein the parental non-human antibody is chimeric.

35. The method of claim 32, wherein the antibody specifically recognizes and binds lipotechoic acid.

36. The method of claim 32, wherein the antibody specifically recognizes and binds human tissue factor.

37. The method of claims 32, wherein the humanized antibody is used as a therapeutic product to treat diseases in humans or animals.

38. The method of claims 32, wherein the humanized antibody is used as a diagnostic product.

39. The method of claim 25, wherein the method further comprises making a humanized single-chain antibody (sc-Fv) from the humanized V domains.

40. The method of claim 25, 26 or 32, wherein the fragment of the humanized antibody is one of F(ab′)₂, Fab′ or Fab.
